(19) United States
(12) Patent Application Publication
(10) Pub. No.: US 2023/0108123 A1
(43) Pub. Date: Apr. 6, 2023

(54) COMPOSITIONS AND METHODS FOR TREATING A NEURODEGENERATIVE OR DEVELOPMENTAL DISORDER

(71) Applicants: The Broad Institute, Inc., Cambridge, MA (US); The Regents of the University of California, Oakland, CA (US)

(72) Inventors: Lindy BARRETT, Cambridge, MA (US); Sara G. SUSCO, Cambridge, MA (US); Eugene YEO, Oakland, CA (US); Stefan AIGNER, Oakland, CA (US); En-Ching LUO, Oakland, CA (US)

(73) Assignees: The Broad Institute, Inc., Cambridge, MA (US); The Regents of the University of California, Oakland, CA (US)

(21) Appl. No.: 17/914,121

(22) PCT Filed: Mar. 25, 2021
(86) PCT No.: PCT/US2021/024082

§ 371 Date: Sep. 23, 2022

Related U.S. Application Data
(60) Provisional application No. 63/000,244, filed on Mar. 26, 2020.

(51) Int. Cl. A61K 38/17; A61P 25/28

(52) U.S. Cl. CPC A61K 38/1709; A61P 25/28; A61K 48/00

(57) ABSTRACT

Described and featured are compositions and methods for treating developmental, neurodevelopmental (e.g., Fragile X syndrome (FXS) or Down syndrome (DS)), or neurodegenerative diseases or disorders (e.g., Alzheimer's disease (AD)) by increasing expression of Fragile X Mental Retardation Protein (FMRP) in patients having or having a propensity to develop such diseases or disorders.

### CROSS REFERENCE TO RELATED APPLICATIONS

This international PCT application claims priority to and benefit of provisional patent application No. 63/000,244, filed on Mar. 26, 2020, the contents of which are incorporated herein by reference in their entirety.

### STATEMENT OF RIGHTS TO INVENTIONS MADE UNDER FEDERALLY SPONSORED RESEARCH

This invention was made with government support under Grant Numbers R01NS075449, R01HG004659, U19MH107367, U54HG007005, R01HD101534 and R21MH109761 awarded by the National Institutes of Health. The government has certain rights in the invention.

### BACKGROUND OF THE INVENTION

Fragile X syndrome (FXS) is the most common inherited cause of intellectual disability and a leading monogenic cause of autism, driven by a trinucleotide repeat expansion in the 5′ UTR of the Fragile X mental retardation 1 (FMR1) gene. This expansion in the FMRI gene leads to epigenetic silencing and loss of the encoded RNA binding protein, Fragile X Mental Retardation Protein (FMRP). While RNA targets of FMRP have been delineated in mouse brain tissue or cells, HEK293T cells, and K562 cells, molecular mechanisms based on these data have failed to translate into effective therapeutic strategies. These targets of FMRP are key targets for therapeutic approaches to FXS, Down syndrome (DS) and Alzheimer's disease (AD), particularly AD associated with DS. Currently, there are no effective treatments for Fragile X syndrome (FXS), Down syndrome (DS) or Alzheimer's disease (AD). Methods of treating such neurodevelopmental or neurodegenerative disorders are urgently required.

### SUMMARY OF THE EMBODIMENTS

The present invention features compositions and methods for treating developmental, neurodevelopmental diseases or disorders (e.g., Fragile X syndrome (FXS), Down syndrome

(DS)), or neurodegenerative diseases or disorders (e.g., Alzheimer's disease (AD)) by increasing expression of Fragile X Mental Retardation Protein (FMRP) in patients having such diseases or disorders.

In one aspect of the present invention, a method for reducing the level of a Dual Specificity Tyrosine Phosphorylation Regulated Kinase 1A (DYRK1A) and/or an amyloid-beta precursor (APP) polypeptide or a polynucleotide encoding such polypeptide in a cell is provided, in which the method involves contacting the cell with an expression vector comprising a polynucleotide sequence encoding a Fragile X mental retardation protein (FMRP) polypeptide or a fragment thereof, thereby decreasing the level of the DYRK1A and/or APP protein or polynucleotide in the cell.

In some embodiments of the method of the above aspect, the cell comprises an increased level of DYRK1A and/or APP. In some embodiments, the level of DYRK1A and/or APP is increased by at least about 10% relative to a reference, such as a normal or non-disease reference. In some embodiments, the increased level of DYRK1A and/or APP is associated with a developmental disorder or neurodegenerative disorder. In some embodiments, the developmental disorder is autism, Fragile X syndrome, or Down syndrome.

In some embodiments, the neurodegenerative disorder is Alzheimer's disease. In some embodiments, the polynucleotide is present in an expression vector. In some embodiments, the expression vector is a lentiviral vector, adenoviral vector, or adeno-associated viral vector. In some embodiments, the cell is a mammalian cell. In some embodiments, the mammal is a rodent, canine, feline, or human. In an embodiment, the mammal is a human. In some embodiments, the cell is in vitro or in vivo.

In another aspect, a method for treating a disease associated with an increase in a DYRK1A and/or APP polypeptide in a subject is provided, in which the method involves administering an effective amount of a Fragile X mental retardation protein (FMRP) polypeptide or fragment thereof or a polynucleotide encoding said polypeptide or a fragment thereof to the subject.

In an embodiment of the method of the above aspect, the level of DYRK1A and/or APP is increased by at least about 10% relative to a normal or non-disease reference. In an embodiment of the method the increased level of DYRK1A and/or APP is associated with a developmental disorder or neurodegenerative disorder. In an embodiment of the method, the developmental disorder is autism, Fragile X syndrome, or Down syndrome. In an embodiment, the developmental disorder is Down syndrome. In an embodiment, Down syndrome is associated with a disease or disorder, such as a seizure disorder or a leukemia. In an embodiment, the neurodegenerative disorder is Alzheimer's disease. In an embodiment, the subject is a mammal. In an embodiment, the mammal is a rodent, canine, feline, or human. In a particular embodiment, the mammal is a human. In an embodiment of the method, the polynucleotide is present in an expression vector. In an embodiment, the expression vector is a lentiviral vector, an adenoviral vector, or an adeno-associated viral vector.

In another aspect, a method of treating a subject having or having a propensity to develop Alzheimer's disease is provided, in which the method involves administering to the subject an expression vector comprising a polynucleotide sequence encoding a Fragile X mental retardation protein (FMRP) polypeptide or a fragment thereof, thereby treating the Alzheimer's disease. In some embodiments, the Alzheimer's disease is associated with at least about a 10% increase in the level of APP in a cell of the subject relative to the level of APP present in a corresponding cell of a control subject, e.g., a subject who does not have Alzheimer's disease. In an embodiment of the method, the subject has Down syndrome. In an embodiment, the subject having Down syndrome has an associated disease or disorder, such as a seizure disorder or a leukemia.

In yet another aspect, a method for treating a subject having or having a propensity to develop Fragile X syndrome is provided, in which the method involves administering to the subject an effective amount of a Fragile X mental retardation protein (FMRP) polypeptide or a fragment thereof or a polynucleotide encoding FMRP or a fragment thereof. In an embodiment of the method, the level of DYRK1A and/or APP in a cell of the subject is increased by at least about 10% relative to the level present in a corresponding cell of a control subject that does not have Fragile X syndrome. In an embodiment of the above-delineated methods, the subject is a mammal. In some embodiments, the mammal is a rodent, canine, feline, or a human. In an embodiment, the subject is a human. In an embodiment of the above-delineated methods, the polynucleotide is present in an expression vector. In an embodiment, the expression vector is a lentiviral vector, adenoviral vector, or adeno-associated viral vector.

In an embodiment of the above-delineated methods, lysine (K)-specific histone demethylase 1A (KDM1A) expression or function is inhibited or reduced, and/or and Huntingtin (HTT) expression or function is increased or enhanced in the cell and/or in the subject. In an embodiment, KDM1A expression or function is reduced or inhibited in the cell and/or in the subject. In an embodiment, HTT expression or function is increased or enhanced in the cell and/or in the subject. In an embodiment, the subject is a human.

In another aspect, a method of treating a disease associated with increased lysine (K)-specific histone demethylase 1A (KDM1A) expression or function and/or with decreased Huntingtin (HTT) expression or function in a subject is provided, in which the method involves administering to the subject an effective amount of a Fragile X mental retardation protein (FMRP) polypeptide or a fragment thereof or a polynucleotide encoding FMRP or a fragment thereof.

In another aspect, a method of treating a disease associated with increased lysine (K)-specific histone demethylase 1A (KDM1A) expression or function in a subject is provided, the method comprising administering to the subject an effective amount of a Fragile X mental retardation protein (FMRP) polypeptide or a fragment thereof or a polynucleotide encoding FMRP or a fragment thereof.

In another aspect, a method of treating a disease associated with decreased Huntingtin (HTT) expression or function in a subject is provided, in which the method involves administering to the subject an effective amount of a Fragile X mental retardation protein (FMRP) polypeptide or a fragment thereof or a polynucleotide encoding FMRP or a fragment thereof.

In an embodiment of the above-delineated methods, the increased KDM1A expression or function and/or the decreased HTT expression or function is associated with a developmental disorder or neurodegenerative disorder. In an embodiment of the methods, the developmental disorder is autism, Fragile X syndrome, or Down syndrome. In an embodiment of the methods, the neurodegenerative disorder is Alzheimer's disease. In an embodiment of the methods, the developmental disorder is Down syndrome. In an embodiment, Down syndrome is associated with a disease or disorder, such as a seizure disorder or a leukemia. In an embodiment of the methods, the subject is a mammal. In an embodiment, the subject is a human. In an embodiment of the methods, the polynucleotide is present in an expression vector. In an embodiment, the expression vector is a lentiviral vector, an adenoviral vector, or an adeno-associated viral vector.

In another aspect, a method of decreasing or reducing the expression of a lysine (K)-specific historic demethylase 1A (KDM1A) polypeptide and/or increasing or enhancing the expression of a Huntingtin (HTT) polypeptide or a polynucleotide encoding such polypeptides in a cell is provided, in which the method involves contacting the cell with an expression vector comprising a polynucleotide sequence encoding a Fragile X mental retardation protein (FMRP) polypeptide or a fragment thereof, thereby decreasing or reducing the expression of the KDM1A or polynucleotide, and/or increasing or enhancing the expression of the HTT polypeptide or polynucleotide, in the cell.

In another aspect, a method of decreasing or reducing the expression of a lysine specific histone demethylase 1A (KDM1A) polypeptide or a polynucleotide encoding such polypeptide in a cell is provided, in which the method involves contacting the cell with an expression vector comprising a polynucleotide sequence encoding a Fragile X mental retardation protein (FMRP) polypeptide or a fragment thereof, thereby decreasing or reducing the expression of the KDM1A polypeptide or polynucleotide in the cell.

In yet another aspect, a method of increasing or enhancing the expression of a Huntingtin (HTT) polypeptide or a polynucleotide encoding such polypeptide in a cell is provided, in which the method involves contacting the cell with an expression vector comprising a polynucleotide sequence encoding a Fragile X mental retardation protein (FMRP) polypeptide or a fragment thereof, thereby increasing or enhancing the expression of the HTT polypeptide or polynucleotide in the cell.

In an embodiment of the methods of the above-delineated aspects, the cell comprises an increased level of KDM1A polypeptide and/or a decreased level of HTT polypeptide or polynucleotide encoding such polypeptide. In an embodiment of the methods of the above-delineated aspects, the level of KDM1A polypeptide or encoding polynucleotide is decreased and/or the level of HTT polypeptide or encoding polynucleotide is increased by at least about 10% relative to a normal or non-disease reference. In an embodiment, an increased level of KDM1A or KDM1A polynucleotide and/or a decreased level of HTT or HTT polynucleotide is associated with a developmental or neurodegenerative disease or disorder. In an embodiment, the developmental disorder is autism, Fragile X syndrome, or Down syndrome. In an embodiment, the developmental disorder is Down syndrome. In an embodiment, Down syndrome is associated with a disease or disorder, such as a seizure disorder or a leukemia. In an embodiment, the neurodegenerative disorder is Alzheimer's disease. In an embodiment, expression vector is a lentiviral vector, an adenoviral vector, or an adeno-associated viral vector. In an embodiment, the cell is a mammalian cell. In an embodiment, the cell is a human cell. In an embodiment, the cell is in vitro or in vivo.

In another aspect, a method of downregulating expression of a KDM1A polypeptide or polynucleotide encoding KDM1A and/or upregulating expression of an HTT polypeptide or polynucleotide encoding HTT in a cell, wherein increased KDM1A expression and/or decreased HTT expression are associated with a disease or disorder is provided, in which the method involves contacting the cell with an expression vector comprising a polynucleotide sequence encoding a Fragile X mental retardation protein (FMRP) polypeptide or a fragment thereof, wherein expression of the KDM1A polypeptide and/or the encoding polynucleotide is downregulated and/or expression of the HTT polypeptide and/or the encoding polynucleotide is upregulated in the cell. In an embodiment of the method, the disease or disorder is a developmental disorder or neurodegenerative disorder. In an embodiment, the developmental disorder is autism, Fragile X syndrome, or Down syndrome. In an embodiment, the developmental disorder is Down syndrome. In an embodiment, Down syndrome is associated with a disease or disorder, such as a seizure disorder or a leukemia.

In an embodiment, the neurodegenerative disorder is Alzheimer's disease. In an embodiment, the expression vector is a lentiviral vector, an adenoviral vector, or an adeno-associated viral vector. In an embodiment, the cell is a mammalian cell. In an embodiment, the cell is a human cell. In an embodiment, the cell is in vitro or in vivo.

In another embodiment, a method of decreasing or reducing the expression of a lysine (K)-specific histone demethylase 1A (KDM1A) polypeptide and/or increasing or enhancing the expression of a Huntingtin (HTT) polypeptide or a polynucleotide encoding such polypeptides in a patient having a developmental or neurodegenerative disease or disorder is provided, in which the method involves administering to a patient a Fragile X mental retardation protein (FMRP) polypeptide or a functional fragment thereof, or an expression vector comprising a polynucleotide sequence encoding a Fragile X mental retardation protein (FMRP) polypeptide or a functional fragment thereof, thereby decreasing or reducing the expression of the KDM1A polypeptide or encoding polynucleotide, and/or increasing or enhancing the expression of the HTT polypeptide or encoding polynucleotide, in the patient. In an embodiment, the patient is a human patient. In an embodiment, the developmental disease or disorder is autism, Fragile X syndrome, or Down syndrome. In an embodiment, the developmental disease or disorder is Down syndrome. In an embodiment, Down syndrome is associated with a disease or disorder, such as a seizure disorder or a leukemia. In an embodiment, the neurodegenerative disease or disorder is Alzheimer's disease.

Compositions and articles defined by the invention were isolated or otherwise manufactured in connection with the examples provided hereinbelow. Other features and advantages of the invention will be apparent from the detailed description and from the claims.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “administering” is meant giving, supplying, providing, delivering, or dispensing a composition, agent, therapeutic and the like to a subject, or applying or bringing the composition and the like into contact with the subject. Administering or administration may be accomplished by any of a number of routes, such as, for example, without limitation, topically, orally, subcutaneously, intramuscularly, intraperitoneally, or intravenously (IV).

By “agent” is meant a polypeptide, nucleic acid molecule, or small molecule, as well as fragments of such agents.

By “alteration” or “modulation” is meant a change (an increase, elevation, or enhancement, or a decrease or reduction) in the expression levels or activity of a gene or polypeptide as detected by standard art known methods such as those described herein. As used herein, an alteration includes a 5% change, a 10% change, a 15% change, a 20% change, a 25% change, or greater, such as a 30% change, a 35% change, a 40% change, or a 50% change or greater change in expression level or activity. In embodiments, the change (increase, etc., or decrease, etc.) is relative to a normal, non-disease, healthy control cell, subject, and the like. Accordingly, an increase, etc. or a decrease, etc. may include an at least 5%, 10%, 15%, 20%, 25%, 30%, 35%, 40%, 45%, 50% or greater (including percentage values therebetween) increase, etc., or decrease, etc. in expression level or activity.

By “ameliorate” is meant decrease, reduce, delay, suppress, attenuate, diminish, arrest, or stabilize the development or progression of a disease, condition, or pathology.

By “amyloid-beta precursor (APP) polypeptide” is meant a polypeptide having at least about 85% or greater amino acid sequence identity to GenBank Reference Sequence: AAW82435.1 or a fragment thereof that is increased in Alzheimer's disease or Downs Syndrome. An exemplary amino acid sequence of APP is provided below:

>AAW82435.1 amyloid beta (A4) precursor protein (protease nexin-II, Alzheimer disease) [Homo sapiens]

MLPGLALLLLAAWTARALEVPTDGNAGLLAEPQIAMFCGR
LNMHMNVQNGKWDSDPSGTKTCIDTKEGILQYCQEVYPEL
QITNVVEANQPVTIQNWCKRGRKQCKTHPHFVIPYRCLVG
EFVSDALLVPDKCKFLHQERMDVCETHLHWHTVAKETCSE
KSTNLHDYGMLLPCGIDKFRGVEFVCCPLAEESDNVDSAD
AEEDDSDVWWGGADTDYADGSEDKVVEVAEEEEVAEVEEE
EADDDEDDEDGDEVEEEAEEPYEEATERTTSIATTTTTTT
ESVEEVVREVCSEQAETGPCRAMISRWYFDVTEGKCAPFF
YGGCGGNRNNFDTEEYCMAVCGSAMSQSLLKTTQEPLARD
PVKLPTTAASTPDAVDKYLETPGDENEHAHFQKAKERLEA
KHRERMSQVMREWEEAERQAKNLPKADKKAVIQHFQEKVE
SLEQEAANERQQLVETHMARVEAMLNDRRRLALENYITAL
QAVPPRPRHVFNMLKKYVRAEQKDRQHTLKHFEHVRMVDP
KKAAQIRSQVMTHLRVIYERMNQSLSLLYNVPAVAEEIQD
EVDELLQKEQNYSDDVLANMISEPRISYGNDALMPSLTET
KTTVELLPVNGEFSLDDLQPWHSFGADSVPANTENEVEPV
DARPAADRGLTTRPGSGLTNIKTEEISEVKMDAEERHDSG
YEVHHQKLVFFAEDVGSNKGAIIGLMVGGVVIATVIVITL
VMLKKKQYTSIHHGVVEVDAAVTPEERHLSKMQQNGYENP
TYKFFEQMQN

By “amyloid-beta precursor (APP) polynucleotide” is meant a polynucleotide encoding an APP polypeptide. The sequence of an exemplary APP polynucleotide is provided at GenBank Reference Sequence: AK312326.1, which is reproduced below:

>AK312326.1 Homo sapiens cDNA, FLJ92638, Homo sapiens amyloid beta (A4) precursor protein (proteasenexin-II, Alzheimer disease) (APP), mRNA

AGAGCAAGGACGCGGCGGATCCCACTCGCACAGCAGCGCA
CTCGGTGCCCCGCGCAGGGTCGCAGATGCTGCCCGGTTTG
GCACTGCTCCTGCTGGCCGCCTGGACGGCTCGGGCGCTGG
AGGTACCCACTGATGGTAATGCTGGCCTGCTGGCTGAACC
CCAGATTGCCATGTTCTGTGGCAGACTGAACATGCACATG
AATGTCCAGAATGGGAAGTGGGATTCAGATCCATCAGGGA
CCAAAACCTGCATTGATACCAAGGAAGGCATCCTGCAGTA
TTGCCAAGAAGTCTACCCTGAACTGCAGATCACCAATGTG
GTAGAAGCCAACCAACCAGTGACCATCCAGAACTGGTGCA
AGCGGGGCCGCAAGCAGTGCAAGACCCATCCCCACTTTGT
GATTCCCTACCGCTGCTTAGTTGGTGAGTTTGTAAGTGAT
GCCCTTCTCGTTCCTGACAAGTGCAAATTCTTACACCAGG
AGAGGATGGATGTTTGCGAAACTCATCTTCACTGGCACAC
CGTCGCCAAAGAGACATGCAGTGAGAAGAGTACCAACTTG
CATGACTACGGCATGTTGCTGCCCTGCGGAATTGACAAGT
TCCGAGGGGTAGAGTTTGTGTGTTGCCCACTGGCTGAAGA
AAGTGACAATGTGGATTCTGCTGATGCGGAGGAGGATGAC
TCGGATGTCTGGTGGGGCGGAGCAGACACAGACTATGCAG
ATGGGAGTGAAGACAAAGTAGTAGAAGTAGCAGAGGAGGA
AGAAGTGGCTGAGGTGGAAGAAGAAGAAGCCGATGATGAC
GAGGACGATGAGGATGGTGATGAGGTAGAGGAAGAGGCTG
AGGAACCCTACGAAGAAGCCACAGAGAGAACCACCAGCAT
TGCCACCACCACCACCACCACCACAGAGTCTGTGGAAGAG
GTGGTTCGAGAGGTGTGCTCTGAACAAGCCGAGACGGGGC
CGTGCCGAGCAATGATCTCCCGCTGGTACTTTGATGTGAC
TGAAGGGAAGTGTGCCCCATTCTTTTACGGCGGATGTGGC
GGCAACCGGAACAACTTTGACACAGAAGAGTACTGCATGG
CCGTGTGTGGCAGCGCCATGTCCCAAAGTTTACTCAAGAC
TACCCAGGAACCTCTTGCCCGAGATCCTGTTAAACTCCCT
ACAACAGCAGCCAGTACCCCTGATGCCGTTGACAAGTATC
TCGAGACACCTGGGGATGAGAATGAACATGCCCATTTCCA
GAAAGCCAAAGAGAGGCTTGAGGCCAAGCACCGAGAGAGA
ATGTCCCAGGTCATGAGAGAATGGGAAGAGGCAGAACGTC
AAGCAAAGAACTTGCCTAAAGCTGATAAGAAGGCAGTTAT
CCAGCATTTCCAGGAGAAAGTGGAATCTTTGGAACAGGAA
GCAGCCAACGAGAGACAGCAGCTGGTGGAGACACACATGG
CCAGAGTGGAAGCCATGCTCAATGACCGCCGCCGCCTGGC
CCTGGAGAACTACATCACCGCTCTGCAGGCTGTTCCTCCT
CGGCCTCGTCACGTGTTCAATATGCTAAAGAAGTATGTCC
GCGCAGAACAGAAGGACAGACAGCACACCCTAAAGCATTT
CGAGCATGTGCGCATGGTGGATCCCAAGAAAGCCGCTCAG
ATCCGGTCCCAGGTTATGACACACCTCCGTGTGATTTATG
AGCGCATGAATCAGTCTCTCTCCCTGCTCTACAACGTGCC
TGCAGTGGCCGAGGAGATTCAGGATGAAGTTGATGAGCTG
CTTCAGAAAGAGCAAAACTATTCAGATGACGTCTTGGCCA
ACATGATTAGTGAACCAAGGATCAGTTACGGAAACGATGC
TCTCATGCCATCTTTGACCGAAACGAAAACCACCGTGGAG
CTCCTTCCCGTGAATGGAGAGTTCAGCCTGGACGATCTCC
AGCCGTGGCATTCTTTTGGGGCTGACTCTGTGCCAGCCAA
CACAGAAAACGAAGTTGAGCCTGTTGATGCCCGCCCTGCT
GCCGACCGAGGACTGACCACTCGACCAGGTTCTGGGTTGA
CAAATATCAAGACGGAGGAGATCTCTGAAGTGAAGATGGA
TGCAGAATTCCGACATGACTCAGGATATGAAGTTCATCAT
CAAAAATTGGTGTTCTTTGCAGAAGATGTGGGTTCAAACA
AAGGTGCAATCATTGGACTCATGGTGGGCGGTGTTGTCAT
AGCGACAGTGATCGTCATCACCTTGGTGATGCTGAAGAAG
AAACAGTACACATCCATTCATCATGGTGTGGTGGAGGTTG
ACGCCGCTGTCACCCCAGAGGAGCGCCACCTGTCCAAGAT
GCAGCAGAACGGCTACGAAAATCCAACCTACAAGTTCTTT
GAGCAGATGCAGAACTAG

The term “effective amount” as used herein refers to the amount of an agent required to ameliorate, reduce, delay, improve, abrogate, abate, diminish, alleviate, or eliminate the symptoms and/or effects of a disease, condition, or pathology relative to an untreated patient, and also relates to a sufficient amount of a pharmacological composition to provide the desired effect. The effective amount of an agent or a composition as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen for use according to knowledge and skill in the art. Such amount is referred to as an “effective” amount.

The phrase “therapeutically effective amount” as used herein, e.g., of a polynucleotide encoding FMRP as disclosed herein, means a sufficient amount of the composition to treat a disorder, at a reasonable benefit/risk ratio applicable to any medical treatment. The term “therapeutically effective amount” therefore refers to an amount of an agent or composition as disclosed herein that is sufficient to, for example, effect a therapeutically or prophylactically significant reduction in a symptom or clinical marker associated with a developmental disorder (e.g., autism, Fragile X syndrome, Down syndrome) or a neurodegenerative disorder (e.g., Alzheimer's disease) when administered to a typical subject who has such a disorder. In one embodiment, an effective amount of a polynucleotide encoding FMRP is the amount required to reduce the level of APP.

An effective amount depends on the type of disease to be treated, the severity of the symptoms, the subject being treated, the age and general condition of the subject, the mode of administration and so forth. Thus, it is not possible to specify the exact “effective amount.” However, for any given case, an appropriate “effective amount” can be determined by one of ordinary skill in the art using only routine experimentation. The efficacy of treatment can be judged by an ordinarily skilled practitioner, for example, efficacy can be assessed in animal or in vitro models of a disease. In one embodiment, an effective amount of an FMRP polypeptide or polynucleotide is an amount that leads to a decrease in DYRK1A or APP levels.

Subjects amenable to treatment by the methods as disclosed herein can be identified by any method to diagnose a disease associated with an increase in DYRK1A or APP, such diseases include Fragile X syndrome, Down syndrome, and Alzheimer's disease. In an embodiment, subjects amenable to treatment by the methods as disclosed herein can further be identified by any method to diagnose a disease associated with an increase in lysine (K)-specific histone demethylase 1A (KDM1A) polypeptide or a decrease in Huntingtin (HTT) polypeptide or a polynucleotide, such diseases include Fragile X syndrome, Down syndrome, and Alzheimer's disease. Methods of diagnosing these conditions are well known by persons of ordinary skill in the art.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

“Detect” refers to identifying the presence, absence, or amount of an analyte, compound, agent, or substance to be detected or determined. In one embodiment, the analyte is DYRK1A and/or APP. In another embodiment, the analyte is KDM1A and/or HTT.

By “disease” is meant any condition, disorder, or pathology that damages or interferes with the normal function of a cell, tissue, or organ. Examples of diseases include diseases associated with increased levels of DYRK1A or APP, or an increased level of KDM1A and/or a decreased level of HTT, such as Fragile X syndrome, Down syndrome, and Alzheimer's disease.

By “Dual Specificity Tyrosine Phosphorylation Regulated Kinase 1A (DYRK1A) polypeptide” is meant a polypeptide having at least about 85% or greater amino acid sequence identity to UniProtKB/Swiss-Prot Reference Sequence: Q13627 or a fragment thereof having protein kinase activity. In one embodiment, a DYRK1A polypeptide catalyzes its autophosphorylation on serine/threonine and tyrosine residues. An exemplary amino acid sequence of DYRK1A is provided below:

>sp|Q13627|DYR1_A HUMAN Dual specificity tyrosine-phosphorylation-regulated kinase 1A OS=Homo sapiens OX=9606 GN=DYRK1A PE=1 SV=2

MHTGGETSACKPSSVRLAPSFSFHAAGLQMAGQMPHSHQY
SDRRQPNISDQQVSALSYSDQIQQPLTNQVMPDIVMLQRR
MPQTFRDPATAPLRKLSVDLIKTYKHINEVYYAKKKRRHQ
QGQGDDSSHKKERKVYNDGYDDDNYDYIVKNGEKWMDRYE
IDSLIGKGSFGQVVKAYDRVEQEWVAIKIIKNKKAFLNQA
QIEVRLLELMNKHDTEMKYYIVHLKRHFMFRNHLCLVFEM
LSYNLYDLLRNTNFRGVSLNLTRKFAQQMCTALLFLATPE
LSIIHCDLKPENILLCNPKRSAIKIVDFGSSCQLGQRIYQ
YIQSRFYRSPEVLLGMPYDLAIDMWSLGCILVEMHTGEPL
FSGANEVDQMNKIVEVLGIPPAHILDQAPKARKFFEKLPD
GTWNLKKTKDGKREYKPPGTRKLHNILGVETGGPGGRRAG
ESGHTVADYLKFKDLILRMLDYDPKTRIQPYYALQHSFFK
KTADEGTNTSNSVSTSPAMEQSQSSGTTSSTSSSSGGSSG
TSNSGRARSDPTHQHRHSGGHFTAAVQAMDCETHSPQVRQ
QFPAPLGWSGTEAPTQVTVETHPVQETTFHVAPQQNALHH
HHGNSSHHHHHHHHHHHHHGQQALGNRTRPRVYNSPTNSS
STQDSMEVGHSHHSMTSLSSSTTSSSTSSSSTGNQGNQAY
QNRPVAANTLDFGQNGAMDVNLTVYSNPRQETGIAGHPTY
QFSANTGPAHYMTEGHLTMRQGADREESPMTGVCVQQSPV
ASS

By “Dual Specificity Tyrosine Phosphorylation Regulated Kinase 1A (DYRK1A) polynucleotide” is meant a nucleic acid molecule encoding a DYRK1A polypeptide. The sequence of an exemplary DYRK1A polynucleotide is provided at GenBank Reference Sequence: U52373.1, which is reproduced below:

>U52373.1 Human serine/threonine kinase MNB (mnb) mRNA, complete cds

GTTATAGTTTTGCCGCTGGACTCTTCCCTCCCTTCCCCCA
CCCCATCAGGATGATATGAGACTTGAAAGAAGACGATGCA
TACAGGAGGAGAGACTTCAGCATGCAAACCTTCATCTGTT
CGGCTTGCACCGTCATTTTCATTCCATGCTGCTGGCCTTC
AGATGGCTGGACAGATGCCCCATTCACATCAGTACAGTGA
CCGTCGCCAGCCAAACATAAGTGACCAACAGGTTTCTGCC
TTATCATATTCTGACCAGATTCAGCAACCTCTAACTAACC
AGGTGATGCCTGATATTGTCATGTTACAGAGGCGGATGCC
CCAAACCTTCCGTGACCCAGCAACTGCTCCCCTGAGAAAA
CTTTCTGTTGACTTGATCAAAACATACAAGCATATTAATG
AGGTTTACTATGCAAAAAAGAAGCGAAGACACCAACAGGG
CCAGGGAGACGATTCTAGTCATAAGAAGGAACGGAAGGTT
TACAATGATGGTTATGATGATGATAACTATGATTATATTG
TAAAAAACGGAGAAAAGTGGATGGATCGTTACGAAATTGA
CTCCTTGATAGGCAAAGGTTCCTTTGGACAGGTTGTAAAG
GCATATGATCGTGTGGAGCAAGAATGGGTTGCCATTAAAA
TAATAAAGAACAAGAAGGCTTTTCTGAATCAAGCACAGAT
AGAAGTGCGACTTCTTGAGCTCATGAACAAACATGACACT
GAAATGAAATACTACATAGTGCATTTGAAACGCCACTTTA
TGTTTCGAAACCATCTCTGTTTAGTTTTTGAAATGCTGTC
CTACAACCTCTATGACTTGCTGAGAAACACCAATTTCCGA
GGGGTCTCTTTGAACCTAACACGAAAGTTTGCGCAACAGA
TGTGCACTGCACTGCTTTTCCTTGCGACTCCAGAACTTAG
TATCATTCACTGTGATCTAAAACCTGAAAATATCCTTCTT
TGTAACCCCAAACGCAGTGCAATCAAGATAGTTGACTTTG
GCAGTTCTTGTCAGTTGGGGCAGAGGATATACCAGTATAT
TCAGAGTCGCTTTTATCGGTCTCCAGAGGTGCTACTGGGA
ATGCCTTATGACCTTGCCATTGATATGTGGTCCCTCGGGT
GTATTTTGGTTGAAATGCACACTGGAGAACCTCTGTTCAG
TGGTGCCAATGAGGTAGATCAGATGAATAAAATAGTGGAA
GTTCTGGGTATTCCACCTGCTCATATTCTTGACCAAGCAC
CAAAAGCAAGAAAGTTCTTTGAGAAGTTGCCAGATGGCAC
TTGGAACTTAAAGAAGACCAAAGATGGAAAACGGGAGTAC
AAACCACCAGGAACCCGTAAACTTCATAACATTCTTGGAG
TGGAAACAGGAGGACCTGGTGGGCGACGTGCTGGGGAGTC
AGGTCATACGGTCGCTGACTACTTGAAGTTCAAAGACCTC
ATTTTAAGGATGCTTGATTATGACCCCAAAACTCGAATTC
AACCTTATTATGCTCTGCAGCACAGTTTCTTCAAGAAAAC
AGCTGATGAAGGTACAAATACAAGTAATAGTGTATCTACA
AGCCCCGCCATGGAGCAGTCTCAGTCTTCGGGCACCACCT
CCAGTACATCGTCAAGCTCAGGTGGCTCATCGGGGACAAG
CAACAGTGGGAGAGCCCGGTCGGATCCGACGCACCAGCAT
CGGCACAGTGGTGGGCACTTCACAGCTGCCGTGCAGGCCA
TGGACTGCGAGACACACAGTCCCCAGGTGCGTCAGCAATT
TCCTGCTCCTCTTGGTTGGTCAGGCACTGAAGCTCCTACA
CAGGTCACTGTTGAAACTCATCCTGTTCAAGAAACAACCT
TTCATGTAGCCCCTCAACAGAATGCATTGCATCATCACCA
TGGTAACAGTTCCCATCATCACCACCACCACCACCACCAT
CACCACCACCATGGACAACAAGCCTTGGGTAACCGGACCA
GGCCAAGGGTCTACAATTCTCCAACGAATAGCTCCTCTAC
CCAAGATTCTATGGAGGTTGGCCACAGTCACCACTCCATG
ACATCCCTGTCTTCCTCAACGACTTCTTCCTCGACATCTT
CCTCCTCTACTGGTAACCAAGGCAATCAGGCCTACCACAA
TCGCCCAGTGGCTGCTAATACCTTGGACTTTGGACAGAAT
GGAGCTATGGACGTTAATTTGACCGTCTACTCCAATCCCC
GCCAAGAGACTGGCATAGCTGGACATCCAACATACCAATT
TTCTGCTAATACAGGTCCTGCACATTACATGACTGAAGGA
CATCTGACGATGAGGCAAGGGGCTGATAGAGAAGAGTCCC
CCATGACAGGAGTTTGTGTGCAACAGAGTCCTGTAGCTAG
CTCGTGACTACATTGAAACTTGAGTTTGTTTCTTGTGTGT
TTTTATAGAAGTGGTGTTTTTT

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “Fragile X Mental Retardation Protein (FMRP or FMR1) polypeptide” is meant a polypeptide having at least about 85% or greater amino acid sequence identity to UniProtKB/Swiss-Prot Reference Sequence: Q06787 or a fragment thereof having RNA binding activity. An exemplary amino acid sequence of FMRP is provided below:

```
    1 meelvvevrg sngafykafv kdvhedsitv
        afennwqpdr qipfhdvrfp ppvgynkdin
  61 esdevevysr anekepccww lakvrmikge
        fyvieyaacd atyneivtie rlrsvnpnkp
121 atkdtfhkik Idvpedlrqm cakeaahkdf
        kkavgafsvt ydpenyqlvi isinevtskr
181 ahmlidmhfr slrtklslim rneeaskqle
        ssrqlasrfh eqfivredlm glaigthgan
241 iqqarkvpgv taidldedtc tfhiygedqd
        avkkarsfle faedviqvpr nlvgkvigkn
301 gkliqeivdk sgvvrvriea eneknvpqee
        eimppnslps nnsrvgpnap eekkhldike
361 nsthfsqpns tkvqrvlvas svvagesqkp
        elkawqgmvp fvfvgtkdsi anatvlldyh
421 Inylkevdql rlerlqideq Irqigassrp
        ppnrtdkeks yvtddgqgmg rgsrpyrnrg
481 hgrrgpgyts gtnseasnas etesdhrdel
        sdwslaptee eresfirrgd grrrggggrg
541 qggrgrgggf kgnddhsrtd nrprnpreak
        grttdgslqi rvdcnnersv htktlqntss
601 egsrlrtgkd rnqkkekpds vdgqqplvng
        vp
```

By “Fragile X Mental Retardation Protein (FMRP or FMR1) polynucleotide” is meant a polynucleotide encoding a FMRP polypeptide. The sequence of an exemplary FMRP polynucleotide is provided at GenBank Reference Sequence: X69962.1, which is reproduced below:

```
      1 acggcgagcg cgggcggcgg cggtgacgga ggcgccgctg ccagggggcg tgcggcagcg
    61 cggcggcggc ggcggcggcg gcggcggcgg aggcggcggc ggcggcggcg gcggcggcgg
  121 aggcggcggc ggcggcggcg gcggcggcgg ctgggcctcg agcgcccgca gcccacctct
  181 cgggggcggg ctcccggcgc tagcagggct gaagagaaga tggaggagct ggtggtggaa
  241 gtgcggggct ccaatggcgc tttctacaag gcatttgtaa aggatgttca tgaagattca
  301 ataacagttg catttgaaaa caactggcag cctgataggc agattccatt tcatgatgtc
  361 agattcccac ctcctgtagg ttataataaa gatataaatg aaagtgatga agttgaggtg
  421 tattccagag caaatgaaaa agagccttgc tgttggtggt tagctaaagt gaggatgata
  481 aagggtgagt tttatgtgat agaatatgca gcatgtgatg caacttacaa tgaaattgtc
  541 acaattgaac gtctaagatc tgttaatccc aacaaacctg ccacaaaaga tactttccat
  601 aagatcaagc tggatgtgcc agaagactta cggcaaatgt gtgccaaaga ggcggcacat
  661 aaggatttta aaaaggcagt tggtgccttt tctgtaactt atgatccaga aaattatcag
  721 cttgtcattt tgtccatcaa tgaagtcacc tcaaagcgag cacatatgct gattgacatg
  781 cactttcgga gtctgcgcac taagttgtct ctgataatga gaaatgaaga agctagtaag
  841 cagctggaga gttcaaggca gcttgcctcg agatttcatg aacagtttat cgtaagagaa
  901 gatctgatgg gtctagctat tggtactcat ggtgctaata ttcagcaagc tagaaaagta
  961 cctggggtca ctgctattga tctagatgaa gatacctgca catttcatat ttatggagag
1021 gatcaggatg cagtgaaaaa agctagaagc tttctcgaat ttgctgaaga tgtaatacaa
1081 gttccaagga acttagtagg caaagtaata ggaaaaaatg gaaagctgat tcaggagatt
1141 gtggacaagt caggagttgt gagggtgagg attgaggctg aaaatgagaa aaatgttcca
1201 caagaagagg aaattatgcc accaaattcc cttccttcca ataattcaag ggttggacct
1261 aatgccccag aagaaaaaaa acatttagat ataaaggaaa acagcaccca tttttctcaa
1321 cctaacagta caaaagtcca gagggtgtta gtggcttcat cagttgtagc aggggaatcc
1381 cagaaacctg aactcaaggc ttggcagggt atggtaccat ttgtttttgt gggaacaaag
1441 gacagcatcg ctaatgccac tgttcttttg gattatcacc tgaactattt aaaggaagta
1501 gaccagttgc gtttggagag attacaaatt gatgagcagt tgcgacagat tggagctagt
1561 tctagaccac caccaaatcg tacagataag gaaaaaagct atgtgactga tgatggtcaa
1621 ggaatgggtc gaggtagtag accttacaga aatagggggc acggcagacg cggtcctgga
1681 tatacttcag gaactaattc tgaagcatca aatgcttctg aaacagaatc tgaccacaga
1741 gacgaactca gtgattggtc attagctcca acagaggaag agagggagag cttcctgcgc
1801 agaggagacg gacggcggcg tggaggggga ggaagaggac aaggaggaag aggacgtgga
1861 ggaggcttca aaggaaacga cgatcactcc cgaacagata atcgtccacg taatccaaga
1921 gaggctaaag gaagaacaac agatggatcc cttcagatca gagttgactg caataatgaa
1981 aggagtgtcc acactaaaac attacagaat acctccagtg aaggtagtcg gctgcgcacg
2041 ggtaaagatc gtaaccagaa gaaagagaag ccagacagcg tggatggtca gcaaccactc
2101 gtgaatggag taccctaaac tgcataattc tgaagttata tttcctatac catttccgta
2161 attcttattc catattagaa aactttgtta ggccaaagac aaatagtagg caagatggca
2221 cagggcatga aatgaacaca aattatgcta agaatttttt attttttggt attggccata
2281 agcaacaatt ttcagatttg cacaaaaaga taccttaaaa tttgaaacat tgcttttaaa
2341 actacttagc acttcagggc agattttagt tttattttct aaagtactga gcagtgatat
2401 tctttgttaa tttggaccat tttcctgcat tgggtgatca ttcaccagta cattctcagt
2461 ttttcttaat atatagcatt tatggtaatc atattagact tctgttttca atctcgtata
2521 gaagtcttca tgaaatgcta tgtcatttca tgtcctgtgt cagtttatgt tttggtccac
2581 ttttccagta ttttagtgga ccctgaaatg tgtgtgatgt gacatttgtc attttcatta
2641 gcaaaaaaag ttgtatgatc tgtgcctttt ttatatcttg gcaggtagga atattatatt
2701 tggatgcaga gttcagggaa gataagttgg aaacactaaa tgttaaagat gtagcaaacc
2761 ctgtcaaaca ttagtacttt atagaagaat gcatgctttc catatttttt tccttacata
2821 aacatcaggt taggcagtat aaagaatagg acttgttttt gtttttgttt tgttgcactg
2881 aagtttgata aatagtgtta ttgagagaga tgtgtaattt ttctgtatag acaggagaag
2941 aaagaactat cttcatctga gagaggctaa aatgttttca gctaggaaca aatcttcctg
3001 gtcgaaagtt agtaggatat gcctgctctt tggcctgatg accaatttta acttagagct
3061 ttttttttta attttgtctg ccccaagttt tgtgaaattt ttcatatttt aatttcaagc
3121 ttattttgga gagataggaa ggtcatttcc atgtatgcat aataatcctg caaagtacag
3181 gtactttgtc taagaaacat tggaagcagg ttaaatgttt tgtaaacttt gaaatatatg
3241 gtctaatgtt taagcagaat tggaaaagac taagatcggt taacaaataa caactttttt
3301 ttcttttttt cttttgtttt ttgaagtgtt ggggtttggt tttgtttttt gagtcttttt
3361 tttttaagtg aaatttattg aggaaaaata tgtgaaggac cttcactcta agatgttata
3421 tttttcttaa aaagtaactc ctagtagggg taccactgaa tctgtacaga gccgtaaaaa
3481 ctgaagttct gcctctgatg tattttgtga gtttgtttct ttgaattttc attttacagt
3541 tacttttcct tgcatacaaa caagcatata aaatggcaac aaactgcaca tgatttcaca
3601 aatattaaaa agtcttttaa aaagtattgc caaacattaa tgttgatttc tagttattta
3661 ttctgggaat gtatagtatt tgaaaacaga aattggtacc ttgcacacat catctgtaag
3721 ctgtttggtt ttaaaatact gtagataatt aaccaaggta gaatgacctt gtaatgtaac
3781 tgctcttggg caatattctc tgtacatatt agcgacaaca gattggattt tatgttgaca
3841 tttgtttggt tatagtgcaa tatattttgt atgcaagcag tttcaataaa gtttgatctt
3901 cctctgctaa attgatgttg atgcaatcct tacaaatgat tgcttttaaa attttaagct
3961 aggaaaagaa atctatagaa agtgttctgt tacaaaatgt aactgttacc attggaaatt
4021 tcacgtcata ggaagttagc ctttatctac ccaactttca agaaggttct ttaataaagc
4081 gaaaactcaa ccaaatggta cttttccaca gtgtaccatt aaaatatgca ctagtctctt
4141 tttacaaggc tgtattcagc aagggcctaa cttgcttaaa gtgtaattac taacttctaa
4201 aactgtactt tgattcacat gttttcaaat ggagttggag ttcattcata ttacaatatt
4261 tgtgtgctaa acgtgtatgt ttttcagttc aaagtcatga tgtttttaaa atcttattaa
4321 agtttcaaaa atctgaagat tgtttatcta gatgtaaatt tt
```

By “Lysine (K)-specific histone demethylase 1A (KDM1A) polypeptide” is meant a 20 polypeptide having at least about 85% or greater amino acid sequence identity to UniProtKB/Swiss-Prot Reference Sequence: 060341 (isoform 1) or a fragment thereof. having lysine (K)-specific histone demethylase 1A activity. An exemplary amino acid sequence of human KDM1A (isoform 1) is provided below:

```
                10 20 30 40 50
MLSGKKAAAA AAAAAAAATG TEAGPGTAGG SENGSEVAAQ PAGLSGPAEV
                60 70 80 90 100
GPGAVGERTP RKKEPPRASP PGGLAEPPGS AGPQAGPTVV PGSATPMETG
              110 120 130 140 150
IAETPEGRRT SRRKRAKVEY REMDESLANI SEDEYYSEEE RNAKAEKEKK
              160 170 180 190 200
LPPPPPQAPP EEENESEPEE PSGVEGAAFQ SRLPHDRMTS QEAACFPDII
              210 220 230 240 250
SGPQQTQKVF LFIRNRTLQL WLDNPKIQLT FEATLQQLEA PYNSDTVLVH
              260 270 280 290 300
RVHSYLERHG LINFGIYKRI KPLPTKKTGE VIIIGSGVSG LAAARQLQSE
              310 320 330 340 350
GMDVTLLEAR DRVGGRVATF RKGNYVADLG AMVVTGLGGN PMAVVSKQVN
              360 370 380 390 400
MELAKIKQKC PLYEANGQAV PKEKDEMVBQ EFNRLLEATS YLSHQLDFNV
              410 420 430 440 450
LNNKPVSLGQ ALEVVIQLQE KHVKDEQIEH WKKIVKTQEE LKELLNKMVN
              460 470 480 490 500
LKEKIKELHQ QYKEASEVKP PRDITAEFLV KSKHRDLTAL CKEYDELAET
              310 520 530 540 550
QGKLEEKLQE LEANPPSDVY LSSRDRQILD WHFANLEFAN ATPLSTLSLK
              560 570 580 590 600
HWDQDDDFEE TGSHLTVRNG YSCVPVALAE GLDIKLNTAV RQVRYTASGC
              610 620 630 640 650
EVIAVNTRST SQTFTYKCDA VLCTLPLGVI KQQPPAVQFV PPLPEWKTSA
              660 670 680 690 700
VQRMGFGNLN KVVLCFDRVF WDPSVNLFGH VGSTTASRGE LFLFWNLYKA
              710 720 730 740 750
PILLALVAGE AAGIMBNISD DVIVGRCLAI LKGIFGSSAV PQPKETVVSR
              760 770 780 790 800
WRADPWARGS YSYVAAGSSC NDYDLMAQPI TPGPSIPGAP QPIPRLFFAG
              810 820 830 840 850
EHTIRNYPAT VHGALLSGLR EAGR1ADQFL GAMYTLPRQA TPGVPAQQSP
SM
```

By “Lysine (K)-specific histone demethylase 1A (KDM1A) polynucleotide” is meant a polynucleotide encoding a KDM1A polypeptide. The sequence of an exemplary human KDM1A polynucleotide is provided at GenBank NCBI Reference Sequence: NM_001009999.3, which is reproduced below:

| | |
|---|---|
| 1 | ggcgcgtgcg tacgcgacgg cggttggcgg cgcgcgggca gcgtgaagcg aggcgaggca |
| 61 | aggcttttcg gacccacgga gcgacagagc gagcggcccc tacggccgtc ggcggcccgg |
| 121 | cggcccgaga tgttatctgg gaagaaggcg gcagccgcgg cggcggcggc tgcagcggca |
| 181 | gcaaccggga cggaggctgg ccctgggaca gcaggcggct ccgagaacgg gtctgaggtg |
| 241 | gccgcgcagc ccgcgggcct gtcgggccca gccgaggtcg ggccgggggc ggtgggggag |
| 301 | cgcacacccc gcaagaaaga gcctccgcgg gcctcgcccc ccgggggcct ggcggaaccg |
| 361 | ccggggtccg cagggcctca ggccggccct actgtcgtgc ctgggtctgc gacccccatg |
| 421 | gaaactggaa tagcagagac tccggagggg cgtcggacca gccggcgcaa gcgggcgaag |
| 481 | gtagagtaca gagagatgga tgaaagcttg gccaacctct cagaagatga gtattattca |
| 541 | gaagaagaga gaaatgccaa agcagagaag gaaaagaagc ttcccccacc accccctcaa |
| 601 | gccccacctg aggaagaaaa tgaaagtgag cctgaagaac catcggggca agcaggagga |
| 661 | cttcaagacg acagttctgg agggtatgga gacggccaag catcaggtgt ggagggcgca |
| 721 | gctttccaga gccgacttcc tcatgaccgg atgacttctc aagaagcagc ctgttttcca |
| 781 | gatattatca gtggaccaca acagacccag aaggtttttc ttttcattag aaaccgcaca |
| 841 | ctgcagttgt ggttggataa tccaaagatt cagctgacat ttgaggctac tctccaacaa |
| 901 | ttagaagcac cttataacag tgatactgtg cttgtccacc gagttcacag ttatttagag |
| 961 | cgtcatggtc ttatcaactt cggcatctat aagaggataa aacccctacc aactaaaaag |
| 1021 | acaggaaagg taattattat aggctctggg gtctcaggct tggcagcagc tcgacagtta |
| 1081 | caaagttttg gaatggatgt cacacttttg gaagccaggg atcgtgtggg tggacgagtt |
| 1141 | gccacatttc gcaaaggaaa ctatgtagct gatcttggag ccatggtggt aacaggtctt |
| 1201 | ggagggaatc ctatggctgt ggtcagcaaa caagtaaata tggaactggc caagatcaag |
| 1261 | caaaaatgcc cactttatga agccaacgga caagctgaca ctgtcaaggt tcctaaagag |
| 1321 | aaagatgaaa tggtagagca agagtttaac cggttgctag aagctacatc ttaccttagt |
| 1381 | catcaactag acttcaatgt cctcaataat aagcctgtgt cccttggcca ggcattggaa |
| 1441 | gttgtcattc agttacaaga gaagcatgtc aaagatgagc agattgaaca ttggaagaag |
| 1501 | atagtgaaaa ctcaggaaga attgaaagaa cttcttaata agatggtaaa tttgaaagag |
| 1561 | aaaattaaag aactccatca gcaatacaaa gaagcatctg aagtaaagcc acccagagat |
| 1621 | attactgccg agttcttagt gaaaagcaaa cacagggatc tgaccgccct atgcaaggaa |
| 1681 | tatgatgaat tagctgaaac acaaggaaag ctagaagaaa aacttcagga gttggaagcg |
| 1741 | aatcccccaa gtgatgtata tctctcatca agagacagac aaatacttga ttggcatttt |
| 1801 | gcaaatcttg aatttgctaa tgccacacct ctctcaactc tctcccttaa gcactgggat |
| 1861 | caggatgatg actttgagtt cactggcagc cacctgacag taaggaatgg ctactcgtgt |
| 1921 | gtgcctgtgg ctttagcaga aggcctagac attaaactga atacagcagt gcgacaggtt |
| 1981 | cgctacacgg cttcaggatg tgaagtgata gctgtgaata cccgctccac gagtcaaacc |
| 2041 | tttatttata aatgcgacgc agttctctgt acccttcccc tgggtgtgct gaagcagcag |
| 2101 | ccaccagccg ttcagtttgt gccacctctc cctgagtgga aaacatctgc agtccaaagg |
| 2161 | atgggatttg gcaaccttaa caaggtggtg ttgtgttttg atcgggtgtt ctgggatcca |
| 2221 | agtgtcaatt tgttcgggca tgttggcagt acgactgcca gcaggggtga gctcttcctc |
| 2281 | ttctggaacc tctataaagc tccaatactg ttggcactag tggcaggaga agctgctggt |
| 2341 | atcatggaaa acataagtga cgatgtgatt gttggccgat gcctggccat tctcaaaggg |
| 2401 | atttttggta gcagtgcagt acctcagccc aaagaaactg tggtgtctcg ttggcgtgct |
| 2461 | gatccctggg ctcggggctc ttattcctat gttgctgcag gatcatctgg aaatgactat |
| 2521 | gatttaatgg ctcagccaat cactcctggc ccctcgattc caggtgcccc acagccgatt |
| 2581 | ccacgactct tctttgcggg agaacatacg atccgtaact acccagccac agtgcatggt |
| 2641 | gctctgctga gtgggctgcg agaagcggga agaattgcag accagttttt gggggccatg |
| 2701 | tatacgctgc ctcgccaggc cacaccaggt gttcctgcac agcagtcccc aagcatgtga |
| 2761 | gacagatgca ttctaaggga agaggcccat gtgcctgttt ctgccatgta aggaaggctc |
| 2821 | ttctagcaat actagatccc actgagaaaa tccaccctgg catctgggct cctgatcagc |
| 2881 | tgatggagct cctgatttga caaaggagct tgcctccttt gaatgaccta gagcacaggg |
| 2941 | aggaacttgt ccattagttt ggaattgtgt tcttcgtaaa gactgaggca agcaagtgct |
| 3001 | gtgaaataac atcatcttag tcccttggtg tgtggggttt ttgttttttt tttatatttt |
| 3061 | gagaataaaa cttcatataa aattg |

By “Huntingtin (HTT) polypeptide” is meant a polypeptide (protein) having at least about 85% or greater amino acid sequence identity to NCBI Reference Sequence: NP_001375421.1 (isoform 1) or a fragment thereof having HTT activity. An exemplary amino acid sequence of human HTT (isoform 1) is provided below:

| | |
|---|---|
| 1 | MATLEKLMKA FESLKSFQQQ QQQQQQQQQQ QQQQQQQQPP PPPPPPPPPQ LPQPPPQAQP |
| 61 | LLPQPQPPPP PPPPPPGPAV AEEPLHRPKK ELSATKKDRV NHCLTICENI VAQSVRNSPE |
| 121 | FQKLLGIAME LFLLCSDDAE SDVRMVADEC LNKVIKALMD SNLPRLQLEL YKEIKKNGAP |
| 181 | RSLRAALWRF AELAHLVRPQ KCRPYLVNLL PCLTRTSKRP EESVQETLAA AVPKIMASFG |
| 241 | NFANDNEIKV LLKAFIANLK SSSPTIRRTA AGSAVSICQH SRRTQYFYSW LLNVLLGLLV |
| 301 | PVEDEHSTLL ILGVLLTLRY LVPLLQQQVK DTSLKGSFGV TRKEMEVSPS AEQLVQVYEL |
| 361 | TLHHTQHQDH NVVTGALELL QQLFRTPPPE LLQTLTAVGG IGQLTAAKEE SGGRSRSGSI |
| 421 | VELIAGGGSS CSPVLSRKQK GKVLLGEEEA LEDDSESRSD VSSSALTASV KDEISGELAA |
| 481 | SSGVSTPGSA GHDIITEQPR SQHTLQADSV DLASCDLTSS ATDGDEEDIL SHSSSQVSAV |
| 541 | PSDPAMDLND GTQASSPISD SSQTTTEGPD SAVTPSDSSE IVLDGTDNQY LGLQIGQPQD |
| 601 | EDEEATGILP DEASEAFRNS SMALQQAHLL KNMSHCRQPS DSSVDKFVLR DEATEPGDQE |
| 661 | NKPCRIKGDI GQSTDDDSAP LVHCVRLLSA SFLLTGGKNV LVPDRDVRVS VKALALSCVG |
| 721 | AAVALHPESF FSKLYKVPLD TTEYPEEQYV SDILNYIDHG DPQVRGATAI LCGTLICSIL |
| 781 | SRSRFHVGDW MGTIRTLTGN TFSLADCIPL LRKTLKDESS VTCKLAGTAV RNCVMSLCSS |
| 841 | SYSELGLQLI IDVLTLRNSS YWLVRTELLE TLAEIDFRLV SFLEAKAENL HRGAHHYTGL |
| 901 | LKLQERVLNN VVIHLLGDED PRVRHVAAAS LIRLVPKLFY KCDQGQADPV VAVARDQSSV |
| 961 | YLKLLMHETQ PPSHFSVSTI TRIYRGYNLL PSITDVTMEN NLSRVIAAVS HELITSTTRA |
| 1021 | LTFGCCEALC LLSTAFPVCI WSLGWHCGVP PLSASDESRK SCTVGMATMI LTLLSSAWFP |
| 1081 | LDLSAHQDAL ILAGNLLAAS APKSLRSSWA SEEEANPAAT KQEEVWPALG DRALVPMVEQ |
| 1141 | LFSHLLKVIN ICAHVLDDVA PGPAIKAALP SLTNPPSLSP IRRKGKEKEP GEQASVPLSP |
| 1201 | KKGSEASAAS RQSDTSGPVT TSKSSSLGSF YHLPSYLKLH DVLKATHANY KVTLDLQNST |
| 1261 | EKFGGFLRSA LDVLSQILEL ATLQDIGKCV EEILGYLKSC FSREPMMATV CVQQLLKTLF |
| 1321 | GTNLASQFDG LSSNPSKSQG RAQRLGSSSV RPGLYHYCFM APYTHFTQAL ADASLRNMVQ |
| 1381 | AEQENDTSGW FDVLQKVSTQ LKTNLTSVTK NRADKNAIHN HIRLFEPLVI KALKQYTTTT |
| 1441 | CVQLQKQVLD LLAQLVQLRV NYCLLDSDQV FIGFVLKQFE YIEVGQFRES EAIIPNIFFF |
| 1501 | LVLLSYERYH SKQIIGIPKI IQLCDGIMAS GRKAVTHAIP ALQPIVHDLF VLRGTNKADA |
| 1561 | GKELETQKEV VVSMLLRLIQ YHQVLEMFIL VLQQCHKENE DKWKRLSRQI ADIILPMLAK |
| 1621 | QQMHIDSHEA LGVLNTLFEI LAPSSLRPVD MLLRSMFVTP NTMASVSTVQ LWISGILAIL |
| 1681 | RVLISQSTED IVLSRIQELS FSPYLISCTV INRLRDGDST STLEEHSEGK QIKNLPEETF |
| 1741 | SRFLLQLVGI LLEDIVTKQL KVEMSEQQHT FYCQELGTLL MCLIHIFKSG MFRRITAAAT |
| 1801 | RLFRSDGCGG SFYTLDSLNL RARSMITTHP ALVLLWCQIL LLVNHTDYRW WAEVQQTPKR |
| 1861 | HSLSSTKLLS PQMSGEEEDS DLAAKLGMCN REIVRRGALI LFCDYVCQNL HDSEHLTWLI |
| 1921 | VNHIQDLISL SHEPPVQDFI SAVHRNSAAS GLFIQAIQSR CENLSTPTML KKTLQCLEGI |
| 1981 | HLSQSGAVLT LYVDRLLCTP FRVLARMVDI LACRRVEMLL AANLQSSMAQ LPMEELNRIQ |
| 2041 | EYLQSSGLAQ RHQRLYSLLD RFRLSTMQDS LSPSPPVSSH PLDGDGHVSL ETVSPDKDWY |
| 2101 | VHLVKSQCWT RSDSALLEGA ELVNRIPAED MNAFMMNSEF NLSLLAPCLS LGMSEISGGQ |
| 2161 | KSALFEAARE VTLARVSGTV QQLPAVHHVF QPELPAEPAA YWSKLNDLFG DAALYQSLPT |
| 2221 | LARALAQYLV VVSKLPSHLH LPPEKEKDIV KFVVATLEAL SWHLIHEQIP LSLDLQAGLD |
| 2281 | CCCLALQLPG LVVSWSSTEF VTHACSLIYC VHFILEAVAV QPGEQLLSPE RRTNTPKAIS |
| 2341 | EEEEEVDPNT QNPKYITAAC EMVAEMVESL QSVLALGHKR NSGVPAFLTP LLRNIIISLA |
| 2401 | RLPLVNSYTR VPPLVWKLGW SPKPGGDFGT AFPEIPVEFL QEKEVFKEFI YRINTLGWTS |
| 2461 | RTQFEETWAT LLGVLVTQPL VMEQEESPPE EDTERTQINV LAVQAITSLV LSAMTVPVAG |
| 2521 | NPAVSCLEQQ PRNKPLKALD TRFGRKLSII RGIVEQEIQA MVSKRENIAT HHLYQAWDPV |
| 2581 | PSLSPATTGA LISHEKLLLQ INPERELGSM SYKLGQVSIH SVWLGNSITP LREEEWDEEE |
| 2641 | EEEADAPAPS SPPTSPVNSR KHRAGVDIHS CSQFLLELYS RWILPSSSAR RTPAILISEV |
| 2701 | VRSLLVVSDL FTERNQFELM YVTLTELRRV HPSEDEILAQ YLVPATCKAA AVLGMDKAVA |
| 2761 | EPVSRLLEST LRSSHLPSRV GALHGVLYVL ECDLLDDTAK QLIPVISDYL LSNLKGIAHC |
| 2821 | VNIHSQQHVL VMCATAFYLI ENYPLDVGPE FSASIIQMCG VMLSGSEEST PSIIYHCALR |
| 2881 | GLERLLLSEQ LSRLDAESLV KLSVDRVNVH SPHRAMAALG LMLTCMYTGK EKVSPGRTSD |
| 2941 | PNPAAPDSES VIVAMERVSV LFDRIRKGFP CEARVVARIL PQFLDDFFPP QDIMNKVIGE |
| 3001 | FLSNQQPYPQ FMATVVYKVF QTLHSTGQSS MVRDWVMLSL SNFTQRAPVA MATWSLSCFF |
| 3061 | VSASTSPWVA AILPHVISRM GKLEQVDVNL FCLVATDFYR HQIEEELDRR AFQSVLEVVA |
| 3121 | APGSPYHRLL TCLRNVHKVT TC |

By “Huntingtin (HTT) polynucleotide” is meant a polynucleotide encoding a HTT polypeptide. The sequence of an exemplary human HTT (variant 1) polynucleotide is provided at NCBI Reference Sequence NM_001388492. 1, which is reproduced below:

| | |
|---|---|
| 1 | gctgccggga cgggtccaag atggacggcc gctcaggttc tgcttttacc tgcggcccag |
| 61 | agccccattc attgccccgg tgctgagcgg cgccgcgagt cggcccgagg cctccgggga |
| 121 | ctgccgtgcc gggcgggaga ccgccatggc gaccctggaa aagctgatga aggccttcga |
| 181 | gtccctcaag tccttccagc agcagcagca gcagcagcag cagcagcagc agcagcagca |
| 241 | gcagcagcag cagcaacagc cgccaccgcc gccgccgccg ccgccgcctc ctcagcttcc |
| 301 | tcagccgccg ccgcaggcac agccgctgct gcctcagccg cagccgcccc cgccgccgcc |
| 361 | cccgccgcca cccggcccgg ctgtggctga ggagccgctg caccgaccaa agaaagaact |
| 421 | ttcagctacc aagaaagacc gtgtgaatca ttgtctgaca atatgtgaaa acatagtggc |
| 481 | acagtctgtc agaaattctc cagaatttca gaaacttctg ggcatcgcta tggaactttt |
| 541 | tctgctgtgc agtgatgacg cagagtcaga tgtcaggatg gtggctgacg aatgcctcaa |
| 601 | caaagttatc aaagctttga tggattctaa tcttccaagg ttacagctcg agctctataa |
| 661 | ggaaattaaa aagaatggtg cccctcggag tttgcgtgct gccctgtgga ggtttgctga |
| 721 | gctggctcac ctggttcggc ctcagaaatg caggccttac ctggtgaacc ttctgccgtg |
| 781 | cctgactcga acaagcaaga gacccgaaga atcagtccag gagaccttgg ctgcagctgt |
| 841 | tcccaaaatt atggcttctt ttggcaattt tgcaaatgac aatgaaatta aggttttgtt |
| 901 | aaaggccttc atagcgaacc tgaagtcaag ctcccccacc attcggcgga cagcggctgg |
| 961 | atcagcagtg agcatctgcc agcactcaag aaggacacaa tatttctata gttggctact |
| 1021 | aaatgtgctc ttaggcttac tcgttcctgt cgaggatgaa cactccactc tgctgattct |
| 1081 | tggcgtgctg ctcaccctga ggtatttggt gcccttgctg cagcagcagg tcaaggacac |
| 1141 | aagcctgaaa ggcagcttcg gagtgacaag gaaagaaatg gaagtctctc cttctgcaga |
| 1201 | gcagcttgtc caggtttatg aactgacgtt acatcataca cagcaccaag accacaatgt |
| 1261 | tgtgaccgga gccctggagc tgttgcagca gctcttcaga acgcctccac ccgagcttct |
| 1321 | gcaaaccctg accgcagtcg ggggcattgg gcagctcacc gctgctaagg aggagtctgg |
| 1381 | tggccgaagc cgtagtggga gtattgtgga acttatagct ggagggggtt cctcatgcag |
| 1441 | ccctgtcctt tcaagaaaac aaaaaggcaa agtgctctta ggagaagaag aagccttgga |
| 1501 | ggatgactct gaatcgagat cggatgtcag cagctctgcc ttaacagcct cagtgaagga |
| 1561 | tgagatcagt ggagagctgg ctgcttcttc aggggtttcc actccagggt cagcaggtca |
| 1621 | tgacatcatc acagaacagc cacggtcaca gcacacactg caggcggact cagtggatct |
| 1681 | ggccagctgt gacttgacaa gctctgccac tgatggggat gaggaggata tcttgagcca |
| 1741 | cagctccagc caggtcagcg ccgtcccatc tgaccctgcc atggacctga atgatgggac |
| 1801 | ccaggcctcg tcgcccatca gcgacagctc ccagaccacc accgaagggc ctgattcagc |
| 1861 | tgttacccct tcagacagtt ctgaaattgt gttagacggt accgacaacc agtatttggg |
| 1921 | cctgcagatt ggacagcccc aggatgaaga tgaggaagcc acaggtattc ttcctgatga |
| 1981 | agcctcggag gccttcagga actcttccat ggcccttcaa caggcacatt tattgaaaaa |
| 2041 | catgagtcac tgcaggcagc cttctgacag cagtgttgat aaatttgtgt tgagagatga |
| 2101 | agctactgaa ccgggtgatc aagaaaacaa gccttgccgc atcaaaggtg acattggaca |
| 2161 | gtccactgat gatgactctg cacctcttgt ccattgtgtc cgccttttat ctgcttcgtt |
| 2221 | tttgctaaca gggggaaaaa atgtgctggt tccggacagg gatgtgaggg tcagcgtgaa |
| 2281 | ggccctggcc ctcagctgtg tgggagcagc tgtggccctc cacccggaat ctttcttcag |
| 2341 | caaactctat aaagttcctc ttgacaccac ggaataccct gaggaacagt atgtctcaga |
| 2401 | catcttgaac tacatcgatc atggagaccc acaggttcga ggagccactg ccattctctg |
| 2461 | tgggaccctc atctgctcca tcctcagcag gtcccgcttc cacgtgggag attggatggg |
| 2521 | caccattaga accctcacag gaaatacatt ttctttggcg gattgcattc ctttgctgcg |
| 2581 | gaaaacactg aaggatgagt cttctgttac ttgcaagtta gcttgtacag ctgtgaggaa |
| 2641 | ctgtgtcatg agtctctgca gcagcagcta cagtgagtta ggactgcagc tgatcatcga |
| 2701 | tgtgctgact ctgaggaaca gttcctattg gctggtgagg acagagcttc tggaaaccct |
| 2761 | tgcagagatt gacttcaggc tggtgagctt tttggaggca aaagcagaaa acttacacag |
| 2821 | aggggctcat cattatacag ggcttttaaa actgcaagaa cgagtgctca ataatgttgt |
| 2881 | catccatttg cttggagatg aagaccccag ggtgcgacat gttgccgcag catcactaat |
| 2941 | taggcttgtc ccaaagctgt tttataaatg tgaccaagga caagctgatc cagtagtggc |
| 3001 | cgtggcaaga gatcaaagca gtgtttacct gaaacttctc atgcatgaga cgcagcctcc |
| 3061 | atctcatttc tccgtcagca caataaccag aatatataga ggctataacc tactaccaag |
| 3121 | cataacagac gtcactatgg aaaataacct ttcaagagtt attgcagcag tttctcatga |
| 3181 | actaatcaca tcaaccacca gagcactcac atttggatgc tgtgaagctt tgtgtcttct |
| 3241 | ttccactgcc ttcccagttt gcatttggag tttaggttgg cactgtggag tgcctccact |
| 3301 | gagtgcctca gatgagtcta ggaagagctg taccgttggg atggccacaa tgattctgac |
| 3361 | cctgctctcg tcagcttggt tcccattgga tctctcagcc catcaagatg ctttgatttt |
| 3421 | ggccggaaac ttgcttgcag ccagtgctcc caaatctctg agaagttcat gggcctctga |
| 3481 | agaagaagcc aacccagcag ccaccaagca agaggaggtc tggccagccc tgggggaccg |
| 3541 | ggccctggtg cccatggtgg agcagctctt ctctcacctg ctgaaggtga ttaacatttg |
| 3601 | tgcccacgtc ctggatgacg tggctcctgg acccgcaata aaggcagcct tgccttctct |
| 3661 | aacaaacccc ccttctctaa gtcccatccg acgaaagggg aaggagaaag aaccaggaga |
| 3721 | acaagcatct gtaccgttga gtcccaagaa aggcagtgag gccagtgcag cttctagaca |
| 3781 | atctgatacc tcaggtcctg ttacaacaag taaatcctca tcactgggga gtttctatca |
| 3841 | tcttccttca tacctcaaac tgcatgatgt cctgaaagct acacacgcta actacaaggt |
| 3901 | cacgctggat cttcagaaca gcacggaaaa gtttggaggg tttctccgct cagccttgga |
| 3961 | tgttctttct cagatactag agctggccac actgcaggac attgggaagt gtgttgaaga |
| 4021 | gatcctagga tacctgaaat cctgctttag tcgagaacca atgatggcaa ctgtttgtgt |
| 4081 | tcaacaattg ttgaagactc tctttggcac aaacttggcc tcccagtttg atggcttatc |
| 4141 | ttccaacccc agcaagtcac aaggccgagc acagcgcctt ggctcctcca gtgtgaggcc |
| 4201 | aggcttgtac cactactgct tcatggcccc gtacacccac ttcacccagg ccctcgctga |
| 4261 | cgccagcctg aggaacatgg tgcaggcgga gcaggagaac gacacctcgg gatggtttga |
| 4321 | tgtcctccag aaagtgtcta cccagttgaa gacaaacctc acgagtgtca caaagaaccg |
| 4381 | tgcagataag aatgctattc ataatcacat tcgtttgttt gaacctcttg ttataaaagc |
| 4441 | tttaaaacag tacacgacta caacatgtgt gcagttacag aagcaggttt tagatttgct |
| 4501 | ggcgcagctg gttcagttac gggttaatta ctgtcttctg gattcagatc aggtgtttat |
| 4561 | tggctttgta ttgaaacagt ttgaatacat tgaagtgggc cagttcaggg aatcagaggc |
| 4621 | aatcattcca aacatctttt tcttcttggt attactatct tatgaacgct atcattcaaa |
| 4681 | acagatcatt ggaattccta aaatcattca gctctgtgat ggcatcatgg ccagtggaag |
| 4741 | gaaggctgtg acacatgcca taccggctct gcagcccata gtccacgacc tctttgtatt |
| 4801 | aagaggaaca aataaagctg atgcaggaaa agagcttgaa acccaaaaag aggtggtggt |
| 4861 | gtcaatgtta ctgagactca tccagtacca tcaggtgttg gagatgttca ttcttgtcct |
| 4921 | gcagcagtgc cacaaggaga atgaagacaa gtggaagcga ctgtctcgac agatagctga |
| 4981 | catcatcctc ccaatgttag ccaaacagca gatgcacatt gactctcatg aagcccttgg |
| 5041 | agtgttaaat acattatttg agattttggc cccttcctcc ctccgtccgg tagacatgct |
| 5101 | tttacggagt atgttcgtca ctccaaacac aatggcgtcc gtgagcactg ttcaactgtg |
| 5161 | gatatcggga attctggcca ttttgagggt tctgatttcc cagtcaactg aagatattgt |
| 5221 | tctttctcgt attcaggagc tctccttctc tccgtattta atctcctgta cagtaattaa |
| 5281 | taggttaaga gatggggaca gtacttcaac gctagaagaa cacagtgaag ggaaacaaat |
| 5341 | aaagaatttg ccagaagaaa cattttcaag gtttctatta caactggttg gtattctttt |
| 5401 | agaagacatt gttacaaaac agctgaaggt ggaaatgagt gagcagcaac atactttcta |
| 5461 | ttgccaggaa ctaggcacac tgctaatgtg tctgatccac atcttcaagt ctggaatgtt |
| 5521 | ccggagaatc acagcagctg ccactaggct gttccgcagt gatggctgtg gcggcagttt |
| 5581 | ctacaccctg gacagcttga acttgcgggc tcgttccatg atcaccaccc acccggccct |
| 5641 | ggtgctgctc tggtgtcaga tactgctgct tgtcaaccac accgactacc gctggtgggc |
| 5701 | agaagtgcag cagaccccga aaagacacag tctgtccagc acaaagttac ttagtcccca |
| 5761 | gatgtctgga gaagaggagg attctgactt ggcagccaaa cttggaatgt gcaatagaga |
| 5821 | aatagtacga agaggggctc tcattctctt ctgtgattat gtctgtcaga acctccatga |
| 5881 | ctccgagcac ttaacgtggc tcattgtaaa tcacattcaa gatctgatca gcctttccca |
| 5941 | cgagcctcca gtacaggact tcatcagtgc cgttcatcgg aactctgctg ccagcggcct |
| 6001 | gttcatccag gcaattcagt ctcgttgtga aaacctttca actccaacca tgctgaagaa |
| 6061 | aactcttcag tgcttggagg ggatccatct cagccagtcg ggagctgtgc tcacgctgta |
| 6121 | tgtggacagg cttctgtgca cccctttccg tgtgctggct cgcatggtcg acatccttgc |
| 6181 | ttgtcgccgg gtagaaatgc ttctggctgc aaatttacag agcagcatgg cccagttgcc |
| 6241 | aatggaagaa ctcaacagaa tccaggaata ccttcagagc agcgggctcg ctcagagaca |
| 6301 | ccaaaggctc tattccctgc tggacaggtt tcgtctctcc accatgcaag actcacttag |
| 6361 | tccctctcct ccagtctctt cccacccgct ggacggggat gggcacgtgt cactggaaac |
| 6421 | agtgagtccg gacaaagact ggtacgttca tcttgtcaaa tcccagtgtt ggaccaggtc |
| 6481 | agattctgca ctgctggaag gtgcagagct ggtgaatcgg attcctgctg aagatatgaa |
| 6541 | tgccttcatg atgaactcgg agttcaacct aagcctgcta gctccatgct taagcctagg |
| 6601 | gatgagtgaa atttctggtg gccagaagag tgcccttttt gaagcagccc gtgaggtgac |
| 6661 | tctggcccgt gtgagcggca ccgtgcagca gctccctgct gtccatcatg tcttccagcc |
| 6721 | cgagctgcct gcagagccgg cggcctactg gagcaagttg aatgatctgt ttggggatgc |
| 6781 | tgcactgtat cagtccctgc ccactctggc ccgggccctg gcacagtacc tggtggtggt |
| 6841 | ctccaaactg cccagtcatt tgcaccttcc tcctgagaaa gagaaggaca ttgtgaaatt |
| 6901 | cgtggtggca acccttgagg ccctgtcctg gcatttgatc catgagcaga tcccgctgag |
| 6961 | tctggatctc caggcagggc tggactgctg ctgcctggcc ctgcagctgc ctggcctctg |
| 7021 | gagcgtggtc tcctccacag agtttgtgac ccacgcctgc tccctcatct actgtgtgca |
| 7081 | cttcatcctg gaggccgttg cagtgcagcc tggagagcag cttcttagtc cagaaagaag |
| 7141 | gacaaatacc ccaaaagcca tcagcgagga ggaggaggaa gtagatccaa acacacagaa |
| 7201 | tcctaagtat atcactgcag cctgtgagat ggtggcagaa atggtggagt ctctgcagtc |
| 7261 | ggtgttggcc ttgggtcata aaaggaatag cggcgtgccg gcgtttctca cgccattgct |
| 7321 | aaggaacatc atcatcagcc tggcccgcct gccccttgtc aacagctaca cacgtgtgcc |
| 7381 | cccactggtg tggaagcttg gatggtcacc caaaccggga ggggattttg gcacagcatt |
| 7441 | ccctgagatc cccgtggagt tcctccagga aaaggaagtc tttaaggagt tcatctaccg |
| 7501 | catcaacaca ctaggctgga ccagtcgtac tcagtttgaa gaaacttggg ccaccctcct |
| 7561 | tggtgtcctg gtgacgcagc ccctcgtgat ggagcaggag gagagcccac cagaagaaga |
| 7621 | cacagagagg acccagatca acgtcctggc cgtgcaggcc atcacctcac tggtgctcag |
| 7681 | tgcaatgact gtgcctgtgg ccggcaaccc agctgtaagc tgcttggagc agcagccccg |
| 7741 | gaacaagcct ctgaaagctc tcgacaccag gtttgggagg aagctgagca ttatcagagg |
| 7801 | gattgtggag caagagattc aagcaatggt ttcaaagaga gagaatattg ccacccatca |
| 7861 | tttatatcag gcatgggatc ctgtcccttc tctgtctccg gctactacag gtgccctcat |
| 7921 | cagccacgag aagctgctgc tacagatcaa ccccgagcgg gagctgggga gcatgagcta |
| 7981 | caaactcggc caggtgtcca tacactccgt gtggctgggg aacagcatca cacccctgag |
| 8041 | ggaggaggaa tgggacgagg aagaggagga ggaggccgac gcccctgcac cttcgtcacc |
| 8101 | acccacgtct ccagtcaact ccaggaaaca ccgggctgga gttgacatcc actcctgttc |
| 8161 | gcagtttttg cttgagttgt acagccgctg gatcctgccg tccagctcag ccaggaggac |
| 8221 | cccggccatc ctgatcagtg aggtggtcag atcccttcta gtggtctcag acttgttcac |
| 8281 | cgagcgcaac cagtttgagc tgatgtatgt gacgctgaca gaactgcgaa gggtgcaccc |
| 8341 | ttcagaagac gagatcctcg ctcagtacct ggtgcctgcc acctgcaagg cagctgccgt |
| 8401 | ccttgggatg gacaaggccg tggcggagcc tgtcagccgc ctgctggaga gcacgctcag |
| 8461 | gagcagccac ctgcccagca gggttggagc cctgcacggc gtcctctatg tgctggagtg |
| 8521 | cgacctgctg gacgacactg ccaagcagct catcccggtc atcagcgact atctcctctc |
| 8581 | caacctgaaa gggatcgccc actgcgtgaa cattcacagc cagcagcacg tactggtcat |
| 8641 | gtgtgccact gcgttttacc tcattgagaa ctatcctctg gacgtagggc cggaattttc |
| 8701 | agcatcaata atacagatgt gtggggtgat gctgtctgga agtgaggagt ccaccccctc |
| 8761 | catcatttac cactgtgccc tcagaggcct ggagcgcctc ctgctctctg agcagctctc |
| 8821 | ccgcctggat gcagaatcgc tggtcaagct gagtgtggac agagtgaacg tgcacagccc |
| 8881 | gcaccgggcc atggcggctc tgggcctgat gctcacctgc atgtacacag gaaaggagaa |
| 8941 | agtcagtccg ggtagaactt cagaccctaa tcctgcagcc cccgacagcg agtcagtgat |
| 9001 | tgttgctatg gagcgggtat ctgttctttt tgataggatc aggaaaggct ttccttgtga |
| 9061 | agccagagtg gtggccagga tcctgcccca gtttctagac gacttcttcc caccccagga |
| 9121 | catcatgaac aaagtcatcg gagagtttct gtccaaccag cagccatacc cccagttcat |
| 9181 | ggccaccgtg gtgtataagg tgtttcagac tctgcacagc accgggcagt cgtccatggt |
| 9241 | ccgggactgg gtcatgctgt ccctctccaa cttcacgcag agggccccgg tcgccatggc |
| 9301 | cacgtggagc ctctcctgct tctttgtcag cgcgtccacc agcccgtggg tcgcggcgat |
| 9361 | cctcccacat gtcatcagca ggatgggcaa gctggagcag gtggacgtga accttttctg |
| 9421 | cctggtcgcc acagacttct acagacacca gatagaggag gagctcgacc gcagggcctt |
| 9481 | ccagtctgtg cttgaggtgg ttgcagcccc aggaagccca tatcaccggc tgctgacttg |
| 9541 | tttacgaaat gtccacaagg tcaccacctg ctgagcgcca tggtgggaga gactgtgagg |
| 9601 | cggcagctgg ggccggagcc tttggaagtc tgcgcccttg tgccctgcct ccaccgagcc |
| 9661 | agcttggtcc ctatgggctt ccgcacatgc cgcgggcggc caggcaacgt gcgtgtctct |
| 9721 | gccatgtggc agaagtgctc tttgtggcag tggccaggca gggagtgtct gcagtcctgg |
| 9781 | tggggctgag cctgaggcct tccagaaagc aggagcagct gtgctgcacc ccatgtgggt |
| 9841 | gaccaggtcc tttctcctga tagtcacctg ctggttgttg ccaggttgca gctgctcttg |
| 9901 | catctgggcc agaagtcctc cctcctgcag gctggctgtt ggcccctctg ctgtcctgca |
| 9961 | gtagaaggtg ccgtgagcag gctttgggaa cactggcctg ggtctccctg gtggggtgtg |
| 10021 | catgccacgc cccgtgtctg gatgcacaga tgccatggcc tgtgctgggc cagtggctgg |
| 10081 | gggtgctaga cacccggcac cattctccct tctctctttt cttctcagga tttaaaattt |
| 10141 | aattatatca gtaaagagat taattttaac gtaactcttt ctatgcccgt gtaaagtatg |
| 10201 | tgaatcgcaa ggcctgtgct gcatgcgaca gcgtccgggg tggtggacag ggcccccggc |
| 10261 | cacgctccct ctcctgtagc cactggcata gccctcctga gcacccgctg acatttccgt |
| 10321 | tgtacatgtt cctgtttatg cattcacaag gtgactggga tgtagagagg cgttagtggg |
| 10381 | caggtggcca cagcaggact gaggacaggc ccccattatc ctaggggtgc gctcacctgc |
| 10441 | agcccctcct cctcgggcac agacgactgt cgttctccac ccaccagtca gggacagcag |
| 10501 | cctccctgtc actcagctga gaaggccagc cctccctggc tgtgagcagc ctccactgtg |
| 10561 | tccagagaca tgggcctccc actcctgttc cttgctagcc ctggggtggc gtctgcctag |
| 10621 | gagctggctg gcaggtgttg ggacctgctg ctccatggat gcatgcccta agagtgtcac |
| 10681 | tgagctgtgt tttgtctgag cctctctcgg tcaacagcaa agcttggtgt cttggcactg |
| 10741 | ttagtgacag agcccagcat cccttctgcc cccgttccag ctgacatctt gcacggtgac |
| 10801 | cccttttagt caggagagtg cagatctgtg ctcatcggag actgccccac ggccctgtca |
| 10861 | gagccgccac tcctatcccc aggccaggtc cctggaccag cctcctgttt gcaggcccag |
| 10921 | aggagccaag tcattaaaat ggaagtggat tctggatggc cgggctgctg ctgatgtagg |
| 10981 | agctggattt gggagctctg cttgccgact ggctgtgaga cgaggcaggg gctctgcttc |
| 11041 | ctcagcccta gaggcgagcc aggcaaggtt ggcgactgtc atgtggcttg gtttggtcat |
| 11101 | gcccgtcgat gttttgggta ttgaatgtgg taagtggagg aaatgttgga actctgtgca |
| 11161 | ggtgctgcct tgagaccccc aagcttccac ctgtccctct cctatgtggc agctggggag |
| 11221 | cagctgagat gtggacttgt atgctgccca catacgtgag ggggagctga aagggagccc |
| 11281 | ctcctctgag cagcctctgc caggcctgta tgaggctttt cccaccagct cccaacagag |
| 11341 | gcctccccca gccaggacca cctcgtcctc gtggcggggc agcaggagcg gtagaaaggg |
| 11401 | gtccgatgtt tgaggaggcc cttaagggaa gctactgaat tataacacgt aagaaaatca |
| 11461 | ccattccgta ttggttgggg gctcctgttt ctcatcctag ctttttcctg gaaagcccgc |
| 11521 | tagaaggttt gggaacgagg ggaaagttct cagaactgtt ggctgctccc cacccgcctc |
| 11581 | ccgcctcccc cgcaggttat gtcagcagct ctgagacagc agtatcacag gccagatgtt |
| 11641 | gttcctggct agatgtttac atttgtaaga aataacactg tgaatgtaaa acagagccat |
| 11701 | tcccttggaa tgcatatcgc tgggctcaac atagagtttg tcttcctctt gtttacgacg |
| 11761 | tgatctaaac cagtccttag caaggggctc agaacacccc gctctggcag taggtgtccc |
| 11821 | ccacccccaa agacctgcct gtgtgctccg gagatgaata tgagctcatt agtaaaaatg |
| 11881 | acttcaccca cgcatataca taaagtatcc atgcatgtgc atatagacac atctataatt |
| 11941 | ttacacacac acctctcaag acggagatgc atggcctcta agagtgcccg tgtcggttct |
| 12001 | tcctggaagt tgactttcct tagacccgcc aggtcaagtt agccgcgtga cggacatcca |
| 12061 | ggcgtgggac gtggtcaggg cagggctcat tcattgccca ctaggatccc actggcgaag |
| 12121 | atggtctcca tatcagctct ctgcagaagg gaggaagact ttatcatgtt cctaaaaatc |
| 12181 | tgtggcaagc acccatcgta ttatccaaat tttgttgcaa atgtgattaa tttggttgtc |
| 12241 | aagttttggg ggtgggctgt ggggagattg cttttgtttt cctgctggta atatcgggaa |
| 12301 | agattttaat gaaaccaggg tagaattgtt tggcaatgca ctgaagcgtg tttctttccc |
| 12361 | aaaatgtgcc tcccttccgc tgcgggccca gctgagtcta tgtaggtgat gtttccagct |
| 12421 | gccaagtgct ctttgttact gtccaccctc atttctgcca gcgcatgtgt cctttcaagg |
| 12481 | ggaaaatgtg aagctgaacc ccctccagac acccagaatg tagcatctga gaaggccctg |
| 12541 | tgccctaaag gacacccctc gcccccatct tcatggaggg ggtcatttca gagccctcgg |
| 12601 | agccaatgaa cagctcctcc tcttggagct gagatgagcc ccacgtggag ctcgggacgg |
| 12661 | atagtagaca gcaataactc ggtgtgtggc cgcctggcag gtggaacttc ctcccgttgc |
| 12721 | ggggtggagt gaggttagtt ctgtgtgtct ggtgggtgga gtcaggcttc tcttgctacc |
| 12781 | tgtgagcatc cttcccagca gacatcctca tcgggctttg tccctccccc gcttcctccc |
| 12841 | tctgcgggga ggacccggga ccacagctgc tggccagggt agacttggag ctgtcctcca |
| 12901 | gaggggtcac gtgtaggagt gagaagaagg aagatcttga gagctgctga gggaccttgg |
| 12961 | agagctcagg atggctcaga cgaggacact cgcttgccgg gcctgggcct cctgggaagg |
| 13021 | agggagctgc tcagaatgcc gcatgacaac tgaaggcaac ctggaaggtt caggggccgc |
| 13081 | tcttccccca tgtgcctgtc acgctctggt gcagtcaaag gaacgccttc ccctcagttg |
| 13141 | tttctaagag cagagtctcc cgctgcaatc tgggtggtaa ctgccagcct tggaggatcg |
| 13201 | tggccaacgt ggacctgcct acggagggtg ggctctgacc caagtggggc ctccttgtcc |
| 13261 | aggtctcact gctttgcacc gtggtcagag ggactgtcag ctgagcttga gctcccctgg |
| 13321 | agccagcagg gctgtgatgg gcgagtcccg gagccccacc cagacctgaa tgcttctgag |
| 13381 | agcaaaggga aggactgacg agagatgtat atttaatttt ttaactgctg caaacattgt |
| 13441 | acatccaaat taaaggaaaa aaatggaaac ca |

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “marker” is meant any protein or polynucleotide that has an alteration in expression level or activity that is associated with a disease, condition, pathology, or disorder. In one embodiment, a marker is APP or DYRK1A. In an embodiment, a marker is KDM1A and/or HTT.

As used herein, “obtaining” as in “obtaining an agent” includes synthesizing, isolating, producing, generating, purchasing, or otherwise acquiring the agent.

The term “pharmaceutically acceptable vehicle” refers to conventional carriers (vehicles), excipients, or diluents that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “reduces” is meant a negative alteration or a reduction of at least 5%, 10%, 15%, 20%, 25%, 30%, 40%, 50%, 75%, 80%, 85%, 90%, 95%, 98%, or 100%. In one embodiment, expression of heterologous FMRP in a cell desirably reduces levels of APP or DYRK1A.

By “reference” is meant a standard or control condition. In some cases, a reference is a standard or control against which a test or unknown is compared, measured, assessed, quantified, or evaluated. For example, a reference cell may be a cell obtained from a normal, healthy subject or individual (or sample thereof), e.g., a subject who does not have a particular disease, disorder, or condition, e.g., FXS or DS. In some cases, a reference cell may be a non-disease, healthy, or normal cell obtained from the same or a different individual who has a given disease or condition. In some cases, a reference cell may be one that is transfected with a control vector or polynucleotide, e.g., a normal, non-mutated or non-disease polynucleotide (gene) or fragment thereof, an empty vector, or a vector comprising a polynucleotide sequence encoding an irrelevant control polypeptide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. A reference sequence includes any of the sequences provided herein. In an embodiment, a reference sequence is a nonmutated, normal sequence, or a sequence obtained or derived from a normal, healthy, and/or non-diseased subject or individual.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 65%, at least 70%, at least 75%, at least 80%, at least 85%, at least 90%, at least 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, a rodent, a bovine, equine, canine, ovine, or feline mammal. In an embodiment, a subject is a human. In an embodiment, the subject is a human patient.

Ranges provided herein are understood to be shorthand for all of the values within the range, inclusive of the first and last stated values. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or greater, consecutively, such as to 100 or greater.

The term “tissue” refers to a group or layer of similarly specialized cells which together perform certain special functions.

The terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated. In an embodiment, a disease or disorder, such as Fragile X syndrome, Down syndrome, Alzheimer's disease, or symptoms thereof, is treated when the level of amyloid-beta precursor (APP) or a polynucleotide encoding APP is reduced (in a cell). In an embodiment, a disease or disorder, such as Fragile X syndrome, Down syndrome, Alzheimer's disease, or symptoms thereof, is treated when the level of Dual Specificity Tyrosine Phosphorylation Regulated Kinase 1A (DYRK1A) or a polynucleotide encoding DYRK1A is reduced (in a cell). In an embodiment, a disease or disorder, such as Fragile X syndrome, Down syndrome, Alzheimer's disease, or symptoms thereof, is treated when the levels of DYRK1A, or a polynucleotide encoding DYRK1A, and the levels of APP polypeptide, or a polynucleotide encoding APP, are reduced (in a cell). In an embodiment, a disease or disorder, such as Fragile X syndrome, Down syndrome, Alzheimer's disease, or symptoms thereof, is treated when the expression or function of KDM1A is inhibited or reduced in a cell. In an embodiment, a disease or disorder, such as Fragile X syndrome, Down syndrome, Alzheimer's disease, or symptoms thereof, is treated when the expression or function of HTT is increased, enhanced, or elevated in a cell. In an embodiment, the disease or disorder is Down syndrome, or the symptoms thereof.

The terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, is susceptible to developing, or has the propensity to develop, a disease, disorder, or condition. In an embodiment, the disease or disorder is as Fragile X syndrome, Down syndrome, Alzheimer's disease, or symptoms thereof.

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1N present schematics, graphs (bar graphs, Venn diagrams, pie chart and Volcano plots) related to high-confidence FMRP RNA targets in hPSCs and neurons defined by eCLIP-seq. FIG. 1A Top, is a schematic of FMRP eCLIP-seq in hPSCs and neurons including cell types and genotypes, cross-linking and immunoprecipitation with FMRP antibodies, enrichment of reads in the IP condition over the SMInput and the strategy used for FMR1^{y/-} peak subtraction from each corresponding FMR1^{y/+} dataset. Bottom, FMRP eCLIP-seq tracks for two example genes, MAP1B (left) and KIF1B (right). Gene diagrams are shown on the top and tracks are shown from four eCLIP-seq libraries: FMR1^{y/+} SMInput, FMR1^{y/+} IP, FMR1^{y/-} SMInput, FMR1^{y/-} IP. Note the increased signal in the FMR1^{y/+} IP condition relative to other conditions. Vertical scale bar for MAP1B=−5 to 200; vertical scale bar for KIF1B=−5 to 65. FIG. 1B is a graph depicting FMRP peak locations for each of the indicated eCLIP-seq datasets including coding sequence (CDS), intron, 5′ UTR, 3′ UTR, and other sequences (i.e., intergenic, noncoding exon, stop codon). FIG. 1C is a graph showing the percentage of individual gene targets from each eCLIP-seq dataset with FMRP binding events in introns. FIG. 1D and FIG. 1E show select Gene Ontology (GO) terms for genes identified from significant peaks in hPSCs (FIG. 1D) or neurons (FIG. 1E) compared to all expressed genes in a given cell type (genes were counted as expressed if they had an average TPM≥1 across five RNA-seq replicates in FMR1^{y/+} cells). Fold enrichment for each GO term is plotted on the x-axis (the degree to which a given GO term is over-represented in the list of targets compared to the complete set of genes expressed in that cell type), with the Bonferroni corrected p-value annotated in each bar calculated using Fisher's exact test. FIG. 1F is a Venn diagram demonstrating the overlap (shown as the number of genes) between all unique FMRP targets identified in neurons (left circle) and FMRP targets in hPSCs (smaller right circle), p=1.29×10⁻²⁷. FIGS. 1G-1 is a Venn diagram demonstrating the overlap (shown as number of genes) between FMRP targets identified in neurons (left circle) and the human homologs of mouse FMRP targets previously identified in mouse brain by Darnell et al., 2011 (smaller right circle), p=1.18×10⁻¹³⁷. Significance was determined by hypergeometric test for over enrichment. FIG. 1G-2 is a Venn diagram showing the overlap (shown as # of genes) between FMRP targets identified in neurons (left circle) and the human homologs of mouse FMRP targets previously identified in CA1 pyramidal neurons by Sawicka et al., 2019 (smaller right circle), p=9.55×10⁻¹³³. FIG. 1H is a pie chart showing the percent of all FMRP neuron targets that were also detectably expressed in fetal brain (95.2%) and not detectably expressed in human fetal brain (4.8%; small slice) based on comparison with the Allen BrainSpan Atlas (where genes with RPKM≥1 were considered expressed). FIGS. 1I-1K illustrate the results of a K-mer analysis and analysis of base pairing probabilities from FMRP eCLIP-seq data do not implicate specific motif or structural feature driving target recognition. FIG. 1I is a dot plot generated by a K-mer analysis for all significant peaks from FMRP neuron (ab17722) eCLIP-seq (x-axis) versus k-mer analysis for intron peaks from FMRP neuron (ab17722) eCLIP-seq (y-axis). Values are z-scores and labels note the six sequences found most frequently across the intron peaks. FIG. 1J is a graph showing base pairing probabilities, as a measure of RNA structuredness, calculated in FMRP neuron (ab17722) intron binding sites of length 50 or less (middle two data sets) and flanking sites (data sets flanking the middle two data sets) for comparison. FIG. 1K is a graph showing base pairing probabilities, as a measure of RNA structuredness, calculated in FMRP neuron (ab17722) intron binding sites (middle two data sets) and flanking sites (data sets flanking the middle two data sets) for comparison for FMRP binding sites of length 200 or less. For FIGS. 1J and 1K, base pairing probability values in flanking or binding regions were averaged across each FMRP binding site. No differences were observed between FMRP binding sites and their flanking regions, indicating a lack of support for a highly structured region that specifically recruits FMRP binding. FIG. 1L and FIG. 1M illustrateVolcano plots showing differentially expressed genes between FMR1^{y/+} and FMR1^{y/-} in hPSCs (FIG. 1L) and neurons (FIG. 1M). Five replicates (independent hPSC wells or independent neuronal differentiations) were used for each genotype and cell type. The Y-axis is −log₁₀ of the adjusted p-value and the x-axis is loge of the fold change. Each gene is shown as not significantly differentially expressed (bottom of legend), significantly (p≤0.05) differentially expressed (middle of legend), or both significantly differentially expressed and an eCLIP-seq target (top of legend). Significance was calculated by Benjamini-Hochberg adjusted Wald test as part of the DEseq2 RNA-seq experiment. FIG. 1N presents a bar graph showing the percent of eCLIP-seq targets that were detected as significantly differentially expressed (bottom portion of leftmost bar) in hPSCs and neurons; 15.2% (p=8.28×10⁻⁵) for hPSCs and 1.4% (p=0.000232) for neurons. The number of eCLIP-seq targets detected as differentially expressed by RNA-seq is indicated for each cell type. Significance was calculated by hypergeometric test for over enrichment (*** p≤0.0005).

FIGS. 2A-2G present blots, schematic diagrams, and graphs which demonstrate that a majority of FMRP binding events in neurons are mediated by the key co-factor Fragile X mental retardation syndrome related 1 protein (FXR1P). FIG. 2A is an image of an FMRP IP-Western blot analysis in neurons showing association with FXR1P. Samples were blotted for FXR1P after immunoprecipitating FMRP from FMR1y/+ neurons (left), IgG from FMR1y/+ neurons (center), and FMRP from FMR1y/− neurons (right). Input, supernatant, and wash lanes are also shown for each immunoprecipitation. FXR1P only appears when immunoprecipitated with FMRP from FMR1y/+ neurons. FIG. 2B is a schematic of FXR1+/+ and FXR1−/− isogenic cell line generation using CRISPR-Cas9. FIG. 2C is an image of a Western blot validation showing expected FXR1P loss in FXR1−/− hPSCs. FIG. 2D comprises a schematic of FMRP eCLIP-seq including cell type and genotypes generated, cross-linking and immunoprecipitation with the FMRP antibody ab17722 (Abcam), enrichment of reads in the IP condition over the SMInput and the strategy used for FMR1y/− peak subtraction. FIG. 2E is a Venn diagram illustrating the overlap (shown as number of genes) between FMRP targets in FXR1−/− neurons (left) and FMRP targets in FXR1+/+ neurons from FIGS. 1F and 1G (right). FIG. 2F is a graph showing peak locations for the FMRP eCLIP-seq dataset generated in FXR1−/− neurons (left) compared to FXR1+/+ neurons (right) from FIG. 1B, including CDS, intron, 5′ UTR, 3′ UTR, and other sequences (i.e., intergenic, noncoding exon, stop codon).

FIGS. 3A-3P present pie charts, graphs, a schematic, bar graphs, an image, a heatmap and pie charts related to results demonstrating that FMRP targets are enriched for key neurodevelopmental disease associated genes. FIG. 3A depicts pie charts that show the percent of genes directly implicated in developmental disorders (DDD, 2017), autism (Satterstrom, 2020), epilepsies (Epi25, 2019) and Down syndrome (HSA21) that are detectably expressed in human neurons (darker gray) or not detectably expressed in human neurons (lighter gray). Genes were counted as expressed if they had an average TPM≥1 across five RNA-seq replicates in FMR1^{y/+} neurons. FIG. 3B depicts pie charts that show the percent of expressed genes implicated in disease from FIG. 3A that are FMRP targets in human neurons (darker gray) or not FMRP targets in human neurons (lighter gray). FIG. 3C is a bar graph showing, for each disease dataset, the fold enrichment for FMRP targets plotted on the y-axis, with the p-value annotated in each bar (Developmental Disorders p=6.82×10⁻⁹; Autism p=5.67×10¹¹; Epilepsy p=0.291; Down syndrome p=3.72×10¹⁵). p-values were calculated by hypergeometric test for over enrichment with Bonferroni correction. FIG. 3D depicts pie charts that show the percent of genes directly implicated in developmental disorders (DDD, 2017), autism (Satterstrom, 2020), epilepsy (Epi25, 2019) and Down syndrome (HSA21) that are detectably expressed in hPSCs (darker shade) or not detectably expressed in hPSCs (lighter shade). Genes were counted as expressed if they had an average TPM≥1 across five RNA-seq replicates in FMR1^{y/+} hPSCs. FIG. 3E depicts pie charts that show the percent of expressed genes implicated in disease from FIG. 3D that are FMRP targets in hPSCs (darker shade) or not FMRP targets in hPSCs (lighter shade). These figures illustrate that FMRP targets are enriched for neurodevelopmental disease associated genes in hPSCs. FIG. 3F is a bar graph showing, for each disease dataset, the fold enrichment for FMRP targets plotted on the y-axis, with the p-value annotated in each bar (Developmental Disorders p=1.62×10⁻⁹; Autism p=1.38×10⁻⁶; Epilepsies p=0.964; Down syndrome p=8.93×10⁻⁵). p-values were calculated by hypergeometric test for over enrichment with Bonferroni correction. FIG. 3G is an ideogram of HSA21 showing all unique FMRP RNA targets identified in neurons only, hPSCs only, or both neurons and hPSCs. Scale bar=10 Mb. FIGS. 3H and 31 demonstrate that cells used for FMRP eCLIP-seq are karyotypically normal. FIG. 3H is a graph illustrating a B allele frequency analysis from SNP array data that demonstrates no chromosomal abnormalities in cells used for FMR1y/+ eCLIP-seq. The plot shows a two-dimensional histogram with 20 vertical bins and one horizontal bin for each megabase. Gray boxes are centromeres. FIG. 3I is an karyotypic image of a cytogenic analysis of G-banded metaphase cells that also shows a normal 46, XY karyotype for cells used in FMR1y/+ eCLIP-seq experiments. FIG. 3J is a heatmap of relative enrichment of RNA binding protein (RBP) binding sites on chromosomes 1-22, X, and Y, which shows that RBPs do not show general preference for transcripts from HSA21 in K562 ENCODE dataset. This includes 120 RBPs with eCLIP data from K562 cells collected as part of the ENCODE Consortium Project. Relative enrichment for each RBP was calculated as the number of replicable binding sites occurring on a given chromosome, adjusted by the transcribed and mappable space of the chromosome and the total number of binding sites detected for the given RBP. FIGS. 3K-3P present the results of analyses of HSA21 enrichment. In FIG. 3K, for the Developmental disorders (DDD, 2017), Autism (Satterstrom, 2020) and Epilepsy (Epi25, 2019) datasets, fold enrichment for HSA21-encoded genes is plotted on the y-axis, with the p-value annotated in each bar (Developmental Disorders p=0.708; Autism p=0.115; Epilepsy p=0.325). All p-values were nonsignificant, calculated by hypergeometric tests for enrichment. FIGS. 3L-3O present bar graphs showing the distribution of targets by chromosome. For FMRP neuron targets (FIG. 3L) and hPSC targets (FIG. 3M), the number of genes expressed from each chromosome in each cell type was established using the TPM cutoff of 1. The number of RNA targets from each chromosome was normalized by this number of expressed genes to account for differences in chromosome size and expression. Significance was determined by hypergeometric test for over-enrichment with Bonferroni correction (HSA21 neuron targets p=2.43×10⁻¹⁴ and HSA21 hPSC targets p=0.0053). FMRP mouse brain targets from Darnell et al 2011 (FIG. 3N) and their human homologs (FIG. 3O); as gene expression could not be calculated, total possible genes from a given chromosome were used as calculated by Ensembl. The gray horizontal line in each graph represents the expected percent of target distribution assuming equal chromosome distribution. FIG. 3P depicts pie charts showing percent of FMRP targets in neurons implicated in developmental disorders, autism, epilepsies and Down syndrome that are FXR1P dependent (darker gray) versus FXR1P independent (lighter gray).

FIGS. 4A-4M a schematic, Volcano plots, dot and bar plots, and bar graphs, which illustrate that FXS and DS converge on shared transcriptional perturbations. FIG. 4A is a schematic of cells used for RNA-seq including isogenic FMR1y/+ and FMR1y/− hPSCs and neurons, as well as isogenic trisomy 21 (T21; DS iPSC A) and euploid control hPSCs and neurons. FIG. 4B is a volcano plot for differentially expressed genes in FXS hPSCs. FIG. 4C is a volcano plot for differentially expressed genes in FXS neurons. FIG. 4D is a volcano plot for differentially expressed genes in DS hPSCs. FIG. 4E is a volcano plot for differentially expressed genes in DS neurons. Shaded boxes indicate genes that have a p-value ≤0.05. Log2 fold change is shown on the x-axis and −log₁₀ adjusted p-value is shown on the y-axis. Positive fold change reflects an increase in DS cells relative to euploid cells. Transcripts that reach significance of p≤0.05 are shown in blue shaded area. Significance was calculated by Benjamini-Hochberg adjusted Wald test as part of a DEseq2 RNA-seq experiment. FIG. 4F is a volcano plot of HSA21-encoded transcripts from DS neurons. Log₂ fold change is shown on the x-axis, with the −log₁₀ of the adjusted p-value shown on the y-axis for all transcripts detected from HSA21. Positive fold change reflects an increase in DS neurons relative to euploid control neurons. Transcripts that reach significance of p≤0.05 are shown as scattered dots. Significance was calculated by Benjamini-Hochberg adjusted Wald test as part of a DEseq2 RNA-seq experiment. Five biological replicates were sequenced for each genotype. FIG. 4G is a volcano plot of HSA21-encoded transcripts from DS hPSCs. Log₂ fold change is shown on the x-axis, with the −log 10 of the adjusted p-value shown on the y-axis for all transcripts detected from HSA21 in hPSCs. Positive fold change reflects an increase in DS hPSCs relative to euploid hPSCs. Transcripts that reach significance of p≤0.05 are shown in grey. FIG. 4H presents box plots as examples of expression patterns for two HSA21-encoded transcripts in hPSCs, Dual Specificity Tyrosine Phosphorylation Regulated Kinase 1A (DYRK1A) (p=2.85×10⁻¹⁸) and Amyloid Beta Precursor Protein (APP) (p=1.88×10⁻⁵³). Transcript per million (TPM) values are shown for five biological replicates per cell type/genotype. FIG. 4I is a graph showing the percentage of eCLIP-seq targets that were detected as significantly differentially expressed by RNA-seq (darker shading; p=8.28×10⁻⁵ for hPSCs and p=0.000232 for neurons). The number of eCLIP-seq targets detected as differentially expressed by RNA-seq is indicated for each cell type. FIG. 4J provides box plots as examples of expression patterns for individual FMRP eCLIP-seq targets from isogenic FMR1y/+ and FMR1y/− datasets including NCAM2 (p=0.000154), CBS, DYRK1A, and PCP4. Transcript per million (TPM) values are shown for five biological replicates per cell type/genotype. FIG. 4K is a bar graph showing the percentages of significant differentially expressed genes (DEGs) detected in the isogenic DS model that were also shared with the isogenic FXS model (darker shading/lower portions of the bars), p=3.19×10⁻³³ for hPSC overlap and p=0.00495 for neuron overlap). FIG. 4L is a bar graph showing the percentage of significant DEGs detected in the FXS models that were also shared with the DS models (darker shading/lower portion of the bars), p=3.19×10⁻³³ for hPSC overlap and p=0.00495 for neuron overlap. For FIGS. 4K and 4L, significance was calculated by hypergeometric test for over enrichment. FIG. 4M provides box plots showing examples of expression patterns for individual genes in FXS and DS models with shared perturbations, including NOVA2, TAC1, NIP2A, SOX11 and TUSC3, which are coordinately regulated at the transcript level in the FXS and DS models; NOVA2 (FXS comparison p=0.00433; DS comparison p=0.000802), TAC1 (FXS comparison p=1.08×10⁻⁴⁹; DS comparison p=3.52×10⁻²⁶), NIP2A (FXS comparison p=8.05×10⁻⁹; DS comparison p=2.92×10⁻⁵), SOX11 (FXS comparison p=0.0253; DS comparison p=0.0058), and TUSC3 (FXS comparison p=0.03; DS comparison p=0.00149). TPM values are shown for 5 biological replicates per cell type/genotype. Significance was calculated by Benjamini-Hochberg adjusted Wald test as part of a DEseq2 RNA-seq experiment. For FIGS. 4G, 7A, 4H, 4J, and 4M, significance was calculated by Benjamini-Hochberg adjusted Wald test. For FIGS. 4I, 4, J, and 4K, significance was calculated by hypergeometric test for over enrichment. Significance is indicated by * p≤0.05, ** p≤0.005 and *** p≤0.0005 relative to the indicated control.

FIGS. 5A-5V present chromosome karyotype images, Western blots, schematic depictions and bar graphs and dot plots, showing that FXS and DS converge on shared proteomic perturbations. FIG. 5A is a karyotype showing the G-banding of the DS iPSC lines and euploid iPSC line acquired from Weick et al., 2013, that confirms trisomy 21 for DS iPSC A. FIG. 5B is a karyotype showing the G-banding of the DS iPSC lines and euploid iPSC line acquired from Weick et al., 2013, that confirms trisomy 21 for DS iPSC B. FIG. 5C is a normal karyotype for the euploid iPSC acquired from Weick et al., 2013. FIG. 5D is an image of a Western blot for FMRP in neurons from FMR1y/+ and FMR1y/− isogenic cell lines, euploid iPSC and DS iPSC A isogenic cell lines, plus additional FXS patient iPSC lines (FXS iPSC A, FXS iPSC B, FXS iPSC C) and an additional DS patient iPSC line (DS iPSC B). The blot for the GAPDH loading control is shown below FMRP. As expected, neurons generated from the FXS patient lines and FMR1y/− line lack FMRP expression. FIGS. 5E-5J present bar graphs showing the quantification of Western blots performed in triplicate demonstrating individual cell line results including three control cell lines (FMR1^{y/+} hESCs, Control iPSC A and control iPSC B), four FXS cell lines (CRISPR-engineered FMR1^{y/-} hESCs plus FXS patient iPSC lines A-C) and two DS cell lines (DS iPSC A and B). Pooled results are shown. Proteins queried include the HSA21-encoded FMRP targets CBS (FIG. 5E), NCAM2 (FIG. 5F), PCP4 (FIG. 5G), DYRK1A (FIG. 5H), APP (FIG. 51) and BACE2 (FIG. 5J). Error bars show SEM and significance between each control and disease sample as calculated by unpaired two-tailed t-test. Significance is indicated by *p≤0.05, ** p≤0.005 and *** p≤0.0005 relative to controls. FIG. 5E presents a graph showing the quantification of a Western blot that queried the HSA21-encoded FMRP target CBS in hPSCs. FIG. 5F presents a graph showing the quantification of a Western blot that queried the HSA21-encoded FMRP target NCAM2 in neurons. FIG. 5G presents a graph showing the quantification of a Western blot that queried the HSA21-encoded FMRP target PCP4 in neurons. FIG. 5H presents a graph showing the quantification of a Western blot that queried the HSA21-encoded FMRP target DYRK1A in neurons. FIG. 5I presents a graph showing the quantification of a Western blot that queried the HSA21-encoded FMRP target APP in hPSCs. FIG. 5J presents a graph showing the quantification of a Western blot that queried the HSA21-encoded FMRP target BACE2 in hPSCs. Cell types used for western blots were determined based on where FMRP bound targets in eCLIP-seq data. In cases where FMRP bound a given target in both hPSCs and neurons, a single cell type was selected for analysis. FIG. 5K presents gene diagrams showing location of corresponding eCLIP-seq peaks for genes queried by western blot analysis. Arrows indicate eCLIP-seq peaks in CDS regions, introns and a non-coding exon. The bar above each diagram is scaled to 50 kb. FIG. 5L is a schematic of a CRISPRa experiment. TRE-dCas9-VPR-eGFP was stably integrated into the AAVS1 safe-harbor locus of the DS iPSC A cell line along with three FMR1 activating gRNAs introduced with a multiplexed piggyBac integration strategy. FIG. 5M is a time-course of doxycycline (dox) treatment and sample collection. FIG. 5N presents a graph quantifying the data observed in the image of a FMRP Western blot performed using an isogenic euploid control iPSC line plus the DS-CRISPRa cell line with indicated treatment conditions. FIG. 5O presents a graph quantifying the data observed in the image of a DYRK1A Western blot performed using an isogenic euploid control iPSC line plus the DS-CRISPRa cell line with indicated treatment conditions. FIG. 5P presents a graph quantifying the data observed in the image of an APP Western blot performed using an isogenic euploid control iPSC line plus the DS-CRISPRa cell line with indicated treatment conditions. For FIGS. 5N, 5O, and 5P, the no dox condition is compared with the 48 hr dox, 120 hr dox and 120 hr off dox conditions (all DS CRISPRa cell line) and the isogenic euploid control is used as a reference point for non-DS expression levels. All western blots were performed in triplicate. Error bars show SEM and significance was calculated by unpaired two-tailed t-test. Significance is indicated by * p≤0.05, ** p≤0.005 relative to the indicated control. FIG. 5Q is a plot showing a quantification of Western blots performed in triplicate for CBS using three control cell lines (FMR1^{y/+} hESCs, Control iPSC lines A and B), four FXS cell lines (CRISPR-engineered FMR1^{y/-} hESCs plus FXS patient iPSC lines A-C) and two DS cell lines (DS iPSC lines A and B). An exemplary Western blot is shown beneath the quantification with the gene diagram showing location of corresponding FMRP eCLIP-seq peaks. Symbols indicate eCLIP-seq peaks in CDS regions (circles), introns (triangles) and a non-coding exon (square). FIG. 5R shows transcript expression patterns for CBS from isogenic FMR1^{y/+} (lighter gray) and FMR1^{y/-} (darker gray) datasets. TPM values are shown for five replicates per genotype. FIG. 5S is a plot showing the quantification of Western blots performed in triplicate for NCAM2 using the same control, FXS and DS cell lines listed in (FIG. 5Q). An exemplary Western blot and corresponding gene diagram with FMRP eCLIP-seq peaks is shown beneath the quantification. FIG. 5T is a plot showing the quantification of Western blots performed in triplicate for NCAM2 using isogenic FMR1^{y/+} and FMR1^{y/-} cell lines extracted from the dataset shown in (FIG. 5S). FIG. 5U is a plot showing transcript expression patterns for NCAM2 from isogenic FMR1^{y/+} (lighter gray) and FMR1^{y/-} (darker gray) datasets (p=0.000154). TPM values are shown for five replicates per genotype. Significance was calculated by Benjamini-Hochberg adjusted Wald test as part of the DEseq2 RNA-seq experiment. FIG. 5V is a plot showing the quantification of Western blots performed in triplicate for PCP4 using the same control FXS and DS cell lines listed in (FIG. 5Q). Exemplary blots and corresponding gene diagrams with FMRP eCLIP-seq peaks are shown beneath the quantification. Cell types used for western blot analysis were selected based on where FMRP bound targets in eCLIP-seq datasets. In cases where FMRP bound a given target in both hPSCs and neurons, a single cell type was selected for analysis. Bars above each gene diagram are scaled to 50 kb. For Western blot quantifications, error bars show SEM and significance between control and disease samples was calculated by unpaired two-tailed t-test. Significance is indicated by *p≤0.05, ** p≤0.005 and *** p≤0.0005 relative to controls.

FIGS. 6A-6D depict summary schematics. FIG. 6A is an illustration of the synthesis of predicted FMRP functions from the literature. FMRP has been implicated in multiple aspects of transcriptional and post-transcriptional processing, which means that individual transcript targets could be impacted at different or multiple stages of processing. FMRP is schematized as a blue oval, gray ovals refer to other proteins, DNA is represented by solid lines and RNA is represented by dashed lines. m6A modifications are shown as red triangles. Previously reported binding motifs are shown in brown. FIG. 6B presents illustrations of a large subset of HSA21-encoded transcripts are bound by FMRP. Under normal conditions, FMRP is thought to play an inhibitory role in RNA processing and there are two copies of HSA21. FIG. 6C is an illustration showing that in the context of FXS, loss of FMRP could lead to increased protein expression through multiple mechanisms of transcript regulation shown in FIG. 6A. Similarly, in the context of DS, an extra copy of HSA21 could lead to increased protein expression. FIG. 6D is an illustration showing that increasing FMRP expression in the context of DS is sufficient to reduce DYRK1A and APP protein expression levels.

FIGS. 7A-7O present a Volcano plot, graphs, shematic diagram, bar graphs, Western blots, and charts related to findings and results showing that FMRP upregulation was sufficient to downregulate the expression of DS-implicated targets. FIG. 7A shows a Volcano plot of all transcripts detected from HSA21 from isogenic euploid control versus DS hPSCs. Loge fold change is shown on the x-axis, with the −log₁₀ of the adjusted p-value shown on the y-axis. Positive fold change reflects an increase in DS relative to euploid control. Transcripts that reach significance of p≤0.05 are shown. Significance was calculated by Benjamini-Hochberg adjusted Wald test as part of the DEseq2 RNA-seq experiment. FIGS. 7B-7D show exemplary graphs of expression patterns for HSA21-encoded transcripts in euploid versus DS including DYRK1A (B; p=2.85×10⁻¹⁸), APP (C; p=1.88×10⁻⁵³) and BACE2 (D; p=0.0048). TPM values are shown for five replicates per genotype. Significance was calculated by Benjamini-Hochberg adjusted Wald test as part of the DEseq 2 RNA-seq experiment. FIGS. 7E-71 relate to transcriptional analyses of isogenic euploid and DS hPSCs and neurons. FIG. 7E presents a schematic of cell lines and genotypes used for RNA-seq analysis. FIG. 7F presents a summary of RNA-seq datasets including: cell type (hPSCs or neurons), number of replicates per cell type/genotype, total number of significantly differentially expressed genes (total DEGs), as well as the total numbers of significantly upregulated DEGs and downregulated DEGs. FIG. 7G illustrates a schematic of the DS CRISPRa experiment. Top: TRE-dCas9-VPR-eGFP was stably integrated into the AAVS1 safe-harbor locus of the DS iPSC A cell line along with three FMR1 activating gRNAs introduced with a multiplexed piggyBac integration strategy. Bottom: time-course of doxycycline (dox) treatment of the DS CRISPRa cell line and sample collection. The “washout” condition in the figure is also considered a “post-treatment” condition. FIGS. 7H-7L show graphs and Western blots of the quantification of FMRP (FIG. 7H), FXR1P (FIG. 7I), DYRK1A (FIG. 7J), APP (FIG. 7K) and BACE2 (FIG. 7L) protein levels from the indicated treatment conditions. The DS (untreated) condition is compared with the 48 hr FMRP, 120 hr FMRP and post-treatment conditions (all DS CRISPRa cell line) and the isogenic euploid control iPSC B was used as a reference point for euploid expression levels. Error bars show SEM and significance was calculated by unpaired two-tailed t-test. All Western blots were performed in triplicate. FIGS. 7M, 7N and 70 present dot plots and Western blots showing quantification of Western blots performed in triplicate for DYRK1A, APP and BACE2, respectively, using control, FXS and DS cell lines (FMR1^{y/+} hESCs, Control iPSC lines A and B, shown in FIG. 5Q). Exemplary Western blots and corresponding gene diagrams with FMRP eCLIP-seq peaks are shown beneath each quantification. Cell types used for Western blot analysis were selected based on where FMRP bound targets in eCLIP-seq datasets. In cases where FMRP bound a given target in both hPSCs and neurons, a single cell type was selected for analysis. Bars above each gene diagram are scaled to 50 kb. For Western blot quantifications, error bars show SEM and significance between control and disease samples was calculated by unpaired two-tailed t-test. Significance is indicated by *p≤0.05, ** p≤0.005 and *** p≤0.0005 relative to controls.

FIGS. 8A-8P present graphs, heatmaps, charts, and diagrams related to transcriptional analyses of FMRP CRISPRa time-course. FIGS. 8A-8C show plosts of transcript expression values across the CRISPRa time-course for FMR1 (FIG. 8A), DYRK1A (FIG. 8B) and APP (FIG. 8C). TPM values are shown for four replicates per condition and significance was calculated by Benjamini-Hochberg adjusted Wald test as part of the DEseq2 RNA-seq experiment. FIGS. 8D-8F present bar graphs and heatmaps, as follows: on the left of these figures is a bar graph showing 3450 genes significantly differentially expressed between euploid and DS cell lines. Those that were also detected as significantly differentially expressed following FMRP CRISPRa are shown in the lighter color, top portion of the bar graph. On the right of the figures are heatmaps showing loge fold change for DEGs significantly changed following FMRP CRISPRa, including those that reversed directionality, at the 48 hr timepoint (FIG. 8D), the 120 hr timepoint (FIG. 8E) and post-treatment (FIG. 8F). FIG. 8G (graphically shows select GO terms for all significant DEGs downregulated in Euploid versus DS and then upregulated upon 120 hr FMRP induction. Fold enrichment for each GO term is plotted on the x-axis, with the Bonferroni corrected p-value annotated in each bar calculated using Fisher's exact test. FIG. 8H diagrammatically shows that from the significant DEGS that reversed directionality at the 120 hr time-point, IPA identified 75 upstream regulators that were also FMRP targets by eCLIP-seq which could potentially account for 28% of the reversed DEGs. FIG. 8I presents heat maps showing activation z-scores for KDM1A and HTT comparing DEGs from Euploid vs DS (untreated), DS vs DS 48 hr FMRP, DS vs DS 120 hr FMRP and DS vs post-treatment conditions, generated using IPA. FIG. 8J presents gene diagrams for KDM1A (top) and HTT (bottom) showing the location of corresponding FMRP eCLIP-seq peaks. Symbols indicate eCLIP-seq peaks in CDS regions (green circles). FIG. 8K presents a chart (top) showing network activation values for KDM1A in each of the DS CRISPRa and FXS dataset comparisons, including: activation z-score (z), p-value of overlap (p), and the number of KDM1A target genes within each comparison (n). Shown at the bottom are KDM1A transcript expression values for the same comparisons. TPM values are shown for four replicates per condition in the Euploid vs DS and DS CRISPRa graphs, and five replicates per condition in the FMR1 graph. Significance was calculated by Benjamini-Hochberg adjusted Wald test as part of the DEseq 2 RNA-seq experiment. FIG. 8L presents a chart (top) showing network activation values for HTT in each of the DS CRISPRa and FXS dataset comparisons, including: activation z-score (z), p-value of overlap (p), and the number of HTT target genes within each comparison (n). Shown at the bottom are HTT transcript expression values for the same comparisons. TPM values are shown for four replicates per condition in the Euploid vs DS and DS CRISPRa graphs, and five replicates per condition in the FMR1 graph. Significance was calculated by Benjamini-Hochberg adjusted Wald test as part of the DEseq2 RNA-seq experiment. FIG. 8M presents examples of specific KDM1A gene networks in the Euploid vs DS condition (top) that were then reversed in the DS vs DS 120 hr FMRP condition (bottom) generated using IPA. Examples of individual genes across the full RNA-seq time-course shown on the right. FIG. 8N presents examples of specific HTT gene networks in the Euploid vs DS condition (top) that were then reversed in the DS vs DS 120 hr FMRP condition (bottom) generated using IPA. Examples of individual genes across the full RNA-seq time-course shown on the right. For all figure panels, significance is indicated by *p≤0.05, ** p≤0.005 and *** p≤0.0005 relative to controls. FIG. 8O presents examples of specific KDM1A gene networks in the Euploid vs DS condition (top) that were then reversed in the DS vs DS 48 hr FMRP condition (bottom) generated using IPA. FIG. 8P presents examples of specific HTT gene networks in the Euploid vs DS condition (top) that were then reversed in the DS vs DS 48 hr FMRP condition (bottom) generated using IPA.

### DETAILED DESCRIPTION OF THE EMBODIMENTS

The present invention features compositions and methods for treating developmental, neurodevelopmental (e.g., Fragile X syndrome (FXS) or Down syndrome (DS)), or neurodegenerative diseases or disorders (e.g., Alzheimer's disease (AD)) by increasing expression of Fragile X Mental Retardation Protein (FMRP) in patients having such diseases or disorders.

The invention is based, at least in part, on the discovery that increasing the level of FMRP is sufficient to significantly reduce protein expression levels of APP and DYRK1A, excess levels of which are associated with neurodevelopmental (e.g., Fragile X syndrome (FXS) or Downs syndrome) and/or neurodegenerative disorders (e.g., Alzheimer's disease (AD)) by increasing expression of Fragile X Mental Retardation Protein (FMRP) in patients having such disorders. The invention is further based on the discovery that increasing the level of Fragile X Mental Retardation Protein (FMRP) also decreases the expression of KDM1A and increases the expression of HTT, as increased expression of KDM1A and decreased levels of HTT are associated with neurodevelopmental (e.g., Fragile X syndrome (FXS), Downs syndrome) and/or neurodegenerative disorders (e.g., Alzheimer's disease (AD)) in patients having such disorders.

As reported in detail below, Fragile X syndrome (FXS), driven by loss of the RNA binding protein FMRP, and Down syndrome (DS), driven by an extra copy of chromosome 21 (HSA21), are two common genetic causes of intellectual disability and autism that lack effective therapies. Defined for the first time herein are the global RNA targets of FMRP in human pluripotent stem cells and human cortical neurons, unexpectedly revealing that FMRP binds a substantial fraction of HSA21 encoded transcripts expressed in these cell types. Using patient cell lines, it was discovered that FXS and DS converge on a set of shared transcriptional and proteomic perturbations; some of the same genes are increased in abundance in both FXS and DS. Remarkably, acute upregulation of endogenous FMRP in DS patient cells through CRISPR activation (CRISPRa) is sufficient to significantly reduce protein expression levels of the key HSA21-encoded targets DYRK1A and APP. These results indicate that FXS and DS converge on a set of shared gene targets through alternative mechanisms, which may facilitate new opportunities for therapeutic intervention.

It was therefore hypothesized that FMRP target analyses in human pluripotent stem cells (hPSCs) and human excitatory cortical neurons would uncover novel facets of FMRP function with relevance to human biology. For the first time the global RNA targets of FMRP were analyzed in both cell types using enhanced crosslinking and immunoprecipitation followed by high-throughput sequencing (eCLIP-seq). In vitro derived cortical neurons have been extensively characterized at molecular and physiological levels and employed in multiple studies of neurodevelopmental disease associated genes. To enhance the specificity of the datasets, eCLIP-seq using isogenic FMR1^{y/-} hPSCs and FMR1^{y/-} neurons was also performed and non-specific binding events were eliminated in each cell type through FMR1^{y/-} peak subtraction. These subtracted datasets provide high-confidence FMRP targets in two human cell types. Unexpectedly, it was discovered that FMRP targets were enriched for chromosome 21 (HSA21)-encoded transcripts, including targets strongly implicated in Down syndrome (DS) disease pathogenesis. DS is driven by a third copy of HSA21 which leads to a myriad of phenotypic effects including intellectual disability, elevated rates of autism, as well as elevated rates of Alzheimer's disease. Although FXS patients (1 in 4,000 males and 1 in 8,000 females) and DS patients (1 in 700) share some cognitive and behavioral deficits, a direct mechanistic connection between these diseases has not previously been reported.

Analyses of multiple FXS and DS patient cell lines revealed a set of shared transcriptional and proteomic perturbations. Moreover, protein expression levels of two key HSA21-encoded FMRP targets, DYRK1A and APP, in DS patient cells were significantly reduced by acutely upregulating endogenous FMRP through CRISPRa. Collectively, these results support a novel model in which the loss of FMRP in FXS and an extra copy of HSA21 in DS converge on a set of shared gene targets through different mechanisms (i.e., through loss of an RNA binding protein and an extra copy of a chromosome, respectively). Shared molecular mechanisms may facilitate new opportunities for therapeutic intervention.

### Fragile X Syndrome (FXS)

In most cases, Fragile X syndrome (FXS) is driven by a tri-nucleotide repeat expansion (>200 CGG repeats) in the 5′UTR of the Fragile X mental retardation 1 (FMR1) gene, which leads to epigenetic silencing and complete loss of the encoded RNA binding protein (RBP) Fragile X mental retardation protein (FMRP). FMRP is well-established as a translational repressor through ribosome stalling, but like many RBPs, it is also implicated in other aspects of transcriptional and translational processing. For example, FMRP has been reported to directly bind chromatin through its tandem Tudor domain to regulate genome stability, associate with RNA targets co-transcriptionally, bind snoRNAs in the nucleus to impact rRNA methylation, modulate RNA splicing and RNA editing, facilitate nuclear RNA export, traffic RNAs within the cytoplasm, and phase separate in cytoplasmic ribonucleoprotein (RNP) granules. In some cases, co-factors have been identified that link FMRP with specific functions such as CYFIP1 and translational repression, ADAR proteins and A-I RNA editing, RBM14 and splicing, or YTHDF2 and N⁶-methyladenosine (m6A) modifications. While multiple transcript features and sequence motifs have been proposed to mediate FMRP target recognition, there is currently no consensus mechanism. Moreover, while FMRP RNA targets have been delineated in a variety of cell and tissue types including adult mouse brain tissue and primary mouse brain cells, HEK293T cells and K562 cells, effective therapeutic strategies have not yet emerged. Notably, multiple studies report gene set overlap between FMRP targets identified from mouse brain and genes implicated in autism, schizophrenia and bipolar disorder, which is suggestive of shared mechanisms. Without intending to be bound by theory, gene set overlap between FXS and other neurodevelopmental diseases may lead to mis-regulation of shared molecules or pathways.

### Down Syndrome (DS)

Down syndrome (DS), which is driven by a third copy of chromosome 21 (HSA21), leads to a multitude of phenotypes that originate during early development, including intellectual disability, as well as elevated rates of autism, Alzheimer's disease, seizure disorders and certain types of leukemias. While no single gene encoded on HSA21 drives DS, prominent gene candidates implicated in specific patient phenotypes include Dual Specificity Tyrosine-(Y)-Phosphorylation Regulated Kinase 1 (DYRK1A) and Amyloid Precursor Protein (APP). DYRK1A is independently implicated in both intellectual disability and autism and its overexpression has been shown to induce DS-related cognitive phenotypes in mouse models, which can then be rescued by DYRK1A normalization. Inhibition of DYRK1A has also been shown to partially rescue DS phenotypes in human cellular models, and clinical trials have attempted to normalize DYRK1A with the goal of improving cognitive function in DS patients. DS is also one of the most common genetic causes of early-onset Alzheimer's disease (AD) and APP has been implicated in acting as a primary driver allele for AD pathogenesis in DS. Other examples of DS-implicated genes include BACE2 (Beta-secretase 2) which cleaves APP into amyloid beta peptide, CBS (Cystathionine Beta-Synthase), which is reportedly necessary and sufficient for induction of a subset of cognitive phenotypes in mouse models, NCAM2 (Neuronal Cell Adhesion Molecule 2), with overexpression in mouse models reportedly leading to decreased synaptic maturation and dendritic spine formation and PCP4 (Purkinje Cell Protein 4), with increased levels associated with differentiation defects and ciliopathies. A direct increase in dosage of these and other genes encoded on HSA21, coupled with their secondary effects, generates the complex neurological and non-neurological phenotypes characteristic of DS.

### DS and FXS Disease Targets

Together, DS (affecting ˜1 in 700 individuals) and FXS (affecting ˜1 in 4,000 males and ˜1 in 8,000 females) are the two most common genetic causes of intellectual disability, with a complex set of distinct as well as overlapping phenotypes. Notably, both diseases are associated with deficits in expressive communication as well as increased rates of autism, seizure disorders, and mental health disorders compared with the general population. By contrast, DS patients are at high risk for early-onset AD, as well as certain types of leukemias, and have dysmorphic features that are not characteristic of the FXS patient population. At the cellular level, both DS and FXS have been associated with alterations in dendritic spine morphology, as well as decreased synaptic plasticity and neurogenesis. However, the large number of potentially perturbed genes in each disease presents a significant challenge for dissecting underlying mechanisms and identifying potential therapeutic opportunities.

As described herein, global, high-confidence FMRP targets were defined and analyzed in human pluripotent stem cells (hPSCs) and in vitro derived human excitatory neurons using enhanced crosslinking and immunoprecipitation followed by high-throughput sequencing (eCLIP-seq).

Although FMRP is canonically associated with translational repression, binding events in both introns and exons of target transcripts were identified, thus supporting a pleiotropic role for FMRP in gene regulation. Additional co-factor analyses revealed that FMRP's autosomal paralog FXR1P was required for a majority of FMRP binding events generally, and intron binding specifically. Moreover, the analyses of FMR1 deficient cells showed that FMRP did not directly regulate transcript expression or stability for a majority of its targets in the assessed cell types.

To identify high priority gene candidates in FXS, FMRP targets were compared with other neurodevelopmental disease datasets, unexpectedly revealing significant enrichment for HSA21-encoded transcripts in both hPSCs and neurons. To assess whether gene set overlap between DS and FXS translated into shared molecule or pathway perturbations, two complimentary approaches were used. First, protein expression levels of specific HSA21-encoded FMRP targets were measured in both DS and FXS patient cells, identifying CBS and NCAM2 as significantly upregulated in both diseases. Second, studies were conducted to examine whether increasing FMRP dosage in the context of DS could potentially modulate target expression, given that many HSA21-encoded transcripts were upregulated in DS and also bound by FMRP. Strikingly, acute upregulation of endogenous FMRP through CRISPR activation (CRISPRa) in DS patient cells was sufficient to significantly reduce protein expression levels of the key DS-implicated genes DYRK1A and APP. Moreover, this strategy reversed one-fifth of all global transcriptional perturbations in DS, with FMRP targets such as KDM1A and HTT mediating a subset of the observed effects.

The KDM1A gene (or polynucleotide) encodes a KDM1A protein, which is a histone demethylase, in particular, a flavin-dependent monoamine oxidase, which can demethylate mono- and di-methylated lysines, specifically histone 3, lysines 4 and 9 (H3K4 and H3K9). KDM1A is an acronym for lysine (K)-specific histone demethylase 1A (KDM1A).

The Huntingtin (HTT) gene (or polynucleotide), also known as the IT15 gene, encodes a huntingtin (HTT) protein, which plays an important role in neurons in the brain and normal embryological development. Mutation(s) in the HTT gene are linked to or associated with Huntington's disease, which is a neurodegenerative disorder characterized by loss of striatal neurons.

As described herein, the global RNA targets of FMRP were defined and analyzed in human pluripotent stem cells (hPSCs) and a human excitatory neuron model through a combination of RNA binding, transcriptional and co-factor analyses. FMRP was shown to bind both introns and exons of target transcripts, consistent with a pleiotropic role in gene regulation. FXR1P was further identified as a key collaborator of FMRP for RNA binding generally and for intron binding specifically. FMRP targets compared with other neurodevelopmental disease datasets revealed unexpected enrichment for HSA21-encoded transcripts in both hPSCs and neurons. This gene set overlap between DS and FXS allowed the identification of specific proteins that were upregulated in the cells of both DS and FXS patient. Moreover, acute upregulation of endogenous FMRP using CRISPRa in DS patient cells was demonstrated to be sufficient to significantly reduce protein levels of key targets and reverse 43% of the global transcriptional perturbations in DS, which further strengthens the relevance of this molecular connection. The analyses described herein demonstrated a novel convergence between DS and FXS in relevant human cell types, which may provide beneficial targets and pathways for therapeutic intervention and treatment of diseases.

Collectively, the findings and analyses described herein (i) delineate FMRP targets and binding mechanisms in two relevant human cell types, (ii) identify novel gene set overlap between the two most common genetic causes of intellectual disability, and (iii) pinpoint specific molecules coordinately perturbed in both DS and FXS patient cells, thus providing an advantageous basis for shared therapeutic strategies.

### Fragile X Mental Retardation Protein (FMRP)

The RNA-binding protein Fragile X Mental Retardation Protein (FMRP) is encoded by the FMRP Translational Regulator 1 (FMR1) gene. Trinucleotide repeat expansion in the 5′ UTR of FMR1 leads to epigenetic silencing and loss of the encoded FMRP. FMRP is a multifunctional RNA-binding protein involved in mRNA splicing, stability, and transport. It can also repress mRNA translation by stalling ribosomal translocation during elongation. As described herein, FMRP in humans targets multiple transcripts encoded on chromosome 21. Further, as shown herein several FMRP targets are dysregulated in the absence of FMRP in both Fragile X syndrome and Down syndrome, thereby demonstrating that these syndromes converge on Shared Transcriptional and Proteomic Perturbations.

### Methods for FMRP-Based Therapy

The present invention provides methods of treating disease and/or disorders or symptoms thereof which comprise administering a therapeutically effective amount of a pharmaceutical composition comprising a compound (e.g., a polynucleotide encoding FMRP or an FMRP polypeptide) to a subject (e.g., a mammal such as a human). Thus, one embodiment is a method of treating a subject suffering from or susceptible to a disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Such treatment will be suitably administered to subjects, particularly humans, suffering from, having, susceptible to, or at risk for a disease, disorder, or symptom thereof. In embodiments, the disease or disorder is a neurodegenerative disease or disorder. Determination of those subjects “at risk” can be made by any objective or subjective determination by a diagnostic test or opinion of a subject or health care provider (e.g., genetic test, enzyme, or protein marker, karyotype, family history, and the like). The compounds herein may be also used in the treatment of any other diseases or disorders in which overexpression of a FMRP target is implicated.

The increased expression, levels (amounts), or activity of FMRP in a cell decreases expression of some FMRP targets (e.g., DYRK1A and APP). In some embodiments, increased expression, levels (amounts), or activity of FMRP in a cell decreases or reduces expression of KMD1A and/or its encoding polynucleotide KMD1A. In some embodiments, increased expression, levels (amounts), or activity of FMRP in a cell increases or enhances expression of HTT and/or its encoding polynucleotide HTT. In some embodiments, the cell is a somatic cell. In some embodiments, cell is a brain cell, e.g., a glial cell, or a neuron. The methods presented herein prevent or treat Down syndrome, Fragile X syndrome, and Alzheimer's disease. Down syndrome and Alzheimer's disease, for example, are characterized by increased expression or activity of DYRK1A and/or APP. Accordingly, the invention provides for the treatment of a variety of diseases and disorders associated with increased expression of targets of FMRP. In some embodiments, the invention provides for the treatment of a variety of diseases and disorders associated with increased expression, levels (amounts), or activity of KMD1A and/or its encoding polynucleotide KMD1A. In some embodiments, the invention provides for the treatment of a variety of diseases and disorders associated with decreased, reduced, or downregulated expression of HTT and/or its encoding polynucleotide HTT. In some embodiments, the invention provides for the treatment of a variety of diseases and disorders associated with increased expression of targets of FMRP that are encoded on chromosome 21. In particular embodiments, the disease or disorder is a neurodegenerative or a neurodevelopmental disease or disorder. For example, increased APP, a FMRP target, has been observed in DS and Alzheimer's disease.

The invention generally features methods of increasing FMRP expression or activity in a subject having or at risk of developing Down syndrome, Fragile X syndrome, or Alzheimer's disease. Therapies provided by the invention include polypeptide therapies and polynucleotide therapies. In on embodiment, the method involves contacting a cell of the subject with FMRP polypeptide, or a functional fragment thereof, thereby increasing the expression or activity of FMRP in the cell. In on embodiment, the method involves contacting a cell of the subject with a polynucleotide encoding FMRP, or a functional or active fragment thereof, thereby increasing the expression or activity of FMRP in the cell. Polynucleotides encoding FMRP or a fragment thereof can be encoded in a plasmid or expression vector. In some embodiments, the polynucleotide encoding FMRP or a functional fragment thereof is an RNA polynucleotide. It will be appreciated that a fragment or portion of the FMRP polypeptide is one that retains the function or activity of the protein.

A FMRP polypeptide or a functional fragment thereof can be provided to a cell in vitro, ex vivo, or in vivo, or administered to a subject and/or to the cells of a subject using methods and procedures for delivering or introducing polypeptides as known and practiced in the art, e.g., via electroporation, liposomes, cell chaperones, cell penetrating particles, nanoparticles or microparticles, and the like. See, e.g., Chau, C. et al., Biochem Soc Trans, 48 (2): 357-365 (2020); Y.-W. Lee et al., Theranostics, 9(11):3280-3292 (2019)).

A polynucleotide encoding FMRP or a functional fragment thereof can be used to express the FMRP protein or a fragment thereof in a subject, such as in the cells of a subject. In an embodiment, a polynucleotide encoding FMRP or a functional fragment thereof can be administered to a subject using methods and procedures for delivering or introducing polynucleotides as known and practiced in the art. Alternatively, cells can be contacted with a polynucleotide encoding FMRP or a functional fragment thereof, and then the cells that express FMRP are administered to the subject.

In one aspect, cells can be transfected with a polynucleotide to express a FMRP protein or a fragment thereof using an ex vivo approach in which cells are removed from a patient, transfected by, e.g., electroporation or lipofection, and re-introduced to the patient. In some embodiments, cells are transduced or infected, rather than transfected, with a viral vector comprising a polynucleotide sequence encoding FMRP or a fragment thereof. In some embodiments, the viral vector is a lentiviral vector, an adenoviral vector, or an adeno-associated viral vector. Other viral vectors are contemplated and are known by those having skill in the art, and the vector used may be dependent on the cell type to be transduced.

In various embodiments, the level, expression, or activity of FMRP in a cell in vivo is transiently increased. In various embodiments, the level, expression, or activity of FMRP in a cell in vivo is stably increased. In various embodiments, this is accomplished by administering an agent (e.g., a polynucleotide (polynucleotide sequence) encoding FMRP or a fragment thereof). In various embodiments, the level, expression, or activity of FMRP in a cell is increased for about 3 months, 2 months, 1 month or less after administration of the agent. In various embodiments, the level, expression, or activity of FMRP in a cell is increased for about 4 weeks, 3 weeks, 2 weeks, 1 week or less after administration of the agent. In various embodiments, the level, expression, or activity of FMRP in a cell is increased for about 7 days, 6 days, 5 days, 4 days, 3 days, 2 days, 1 day or less after administration of the agent. In various embodiments, the level, expression, or activity of FMRP in a cell is increased for about 24, 23, 22, 21, 20, 19, 18, 17, 16, 15, 14, 13, 12, 11, 10, 9, 8, 7, 6, 5, 4, 3, 2, or 1 hour or less after administration of the agent. In various embodiments, the level, expression, or activity of FMRP in a cell is increased for about 60, 45, 30, 15, 10, 9, 8, 7, 6, 5, 4, 3, 2, or 1 minute or less after administration of the agent. In various embodiments, the increase in the level, expression, or activity of FMRP in a cell is not constitutive.

The invention also provides methods of binding an FMRP target in a cell. The method involves contacting the cell with a FMRP polypeptide or a polynucleotide encoding FMRP. In some embodiments, binding to the FMRP target is increased when the FMRP polypeptide is is heterodimerized with a Fragile X mental retardation syndrome related 1 protein (FXR1P). In some embodiments, the polynucleotide encoding FMRP also encodes FXR1P. In some embodiments, the polynucleotide of the present invention encodes a fragment of FMRP and/or a fragment of FXR1P. In some embodiments, FXR1P is encoded on a second polynucleotide. Thus, in one embodiment of a method of binding an FMRP target in a cell, the method comprises contacting the cell with a first polynucleotide encoding FMRP and a second nucleotide encoding FXR1P.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of polynucleotides, oligonucleotides, or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference). Polynucleotides may be delivered via transfection or transduction as described supra.

Polynucleotides encoding FMRP or a fragment thereof can be administered to a target tissue in vivo as a single dose or in multiple doses (e.g., sequentially). The expression desired for FMRP in vivo can be tailored by altering the frequency of administration and/or the amount of the polynucleotide encoding the protein or a fragment thereof administered to a subject or used to contact a cell. Because the polynucleotide encoding FMRP administered to a subject is degraded over time, one of skill in the art can remove or stop the in vivo protein expression by halting further administrations and permitting degradation of the polynucleotide encoding FMRP.

### Transfection Reagents

In certain embodiments of the aspects described herein, a polynucleotide encoding FMRP can be introduced into a target tissue in vivo by transfection or lipofection. Suitable agents for transfection or lipofection include, for example but are not limited to, calcium phosphate, DEAE dextran, lipofectin, lipofectamine, DIMRIE C™, SUPERFECT™, and EFFECTIN™ (QIAGEN™), UNIFECTIN™, MAXIFECTIN™, DOTMA, DOGS™ (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

A FMRP polypeptide or polynucleotide can be transfected into a target tissue in vivo as disclosed herein as a complex with cationic lipid carriers (e.g., OLIGOFECTAMINE™) or non-cationic lipid-based carriers (e.g., TRANSIT-TKOTM™, Mirus Bio LLC, Madison, Wis.). Successful introduction of a FMRP polypeptide or polynucleotide into a target tissue in vivo can be monitored using various known methods. Successful transfection of a target tissue in vivo with a FMRP polypeptide or polynucleotide can be determined by measuring the protein expression level of the target polypeptide by e.g., Western blotting or immunocytochemistry.

In some embodiments of the aspects described herein, the FMRP polypeptide or polynucleotide is introduced into a target tissue in vivo using a transfection reagent. Some exemplary transfection reagents include, for example, cationic lipids, such as lipofectin (Junichi et al, U.S. Pat. No. 5,705,188), cationic glycerol derivatives, and polycationic molecules, such as polylysine (Lollo et al., PCT Application WO 97/30731). Examples of commercially available transfection reagents include, for example, LIPOFECTAMINE™ (Invitrogen; Carlsbad, Calif.), LIPOFECTAMINE2000™ (Invitrogen; Carlsbad, Calif.), 293FECTIN™ (Invitrogen; Carlsbad, Calif.), CELLFECTIN™ (Invitrogen; Carlsbad, Calif.), DIVIRIE-C™ (Invitrogen; Carlsbad, Calif.), FREESTYLE™ MAX (Invitrogen; Carlsbad, Calif.), LIPOFECTAMINE™ 2000 CD (Invitrogen; Carlsbad, Calif.), RNAiMAX

(Invitrogen; Carlsbad, Calif.), OLIGOFECTAMINE™ (Invitrogen; Carlsbad, Calif.), OPTIFECT™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), TRANSFECTAM® Reagent (Promega; Madison, Wis.), TransFast™ Transfection Reagent (Promega; Madison, Wis.), TFX™-20 Reagent (Promega; Madison, Wis.), TFX™-50 Reagent (Promega; Madison, Wis.), DREAMFECT™ (OZ Biosciences; Marseille, France), EcoTransfect (OZ Biosciences; Marseille, France), TransPass.sup.a D1 Transfection Reagent (New England Biolabs; Ipswich, Mass., USA), LYOVEC™/LIPOGEN™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TROGANPORTERT™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HIFECT™ (B-Bridge International, Mountain View, Calif., USA), among others.

In other embodiments, highly branched organic compounds, termed “dendrimers,” can be used to bind the exogenous nucleic acid, such as the FMRP polynucleotide described herein, and introduce it into a target tissue in vivo.

In other embodiments, cell penetrating peptides can be used to bind and transport the FMRP polynucleotide described herein, into a target tissue in vivo.

In other embodiments of the aspects described herein, non-chemical methods of transfection are contemplated. Such methods include, but are not limited to, electroporation (methods whereby an instrument is used to create micro-sized holes transiently in the plasma membrane of cells under an electric discharge), sono-poration (transfection via the application of sonic forces to cells), and optical transfection (methods whereby a tiny (about 1 μm diameter) hole is transiently generated in the plasma membrane of a cell using a highly focused laser). In other embodiments, particle-based methods of transfections are contemplated, such as the use of a gene gun, whereby the nucleic acid is coupled to a nanoparticle of an inert solid (commonly gold) which is then “shot” directly into the target cell's nucleus; “magnetofection,” which refers to a transfection method, that uses magnetic force to deliver exogenous nucleic acids coupled to magnetic nanoparticles into target cells; “impalefection,” which is carried out by impaling cells by elongated nanostructures, such as carbon nanofibers or silicon nanowires which have been coupled to exogenous nucleic acids.

Other agents may be utilized to enhance the penetration of the administered nucleic acids, including glycols, such as ethylene glycol and propylene glycol, pyrrols such as 2-pyrrol, azones, and terpenes, such as limonene and menthone.

### Delivery Formulations and Pharmaceutical Compositions

In some embodiments, a FMRP polypeptide or fragment thereof or a polynucleotide encoding a FMRP polypeptide or fragment thereof is delivered to a target tissue in vivo encapsulated in a nanoparticle. Methods for nanoparticle packaging are well known in the art, and are described, for example, in Bose S, et al., J. Virol. 78:8146 (2004); Dong Y et al., Biomaterials 26:6068 (2005); Lobenberg R. et al., J Drug Target 5:171 (1998); Sakuma S R et al., Int J Pharm 177:161 (1999); Virovic L et al., Expert Opin Drug Deliv 2:707 (2005); and Zimmermann E et al., Eur J Pharm Biopharm 52:203 (2001). In some embodiments, where the composition comprises more than one FMRP polypeptide or FMRP polynucleotide, each FMRP polypeptide or polynucleotide is formulated as its own nanoparticle formulation and the pharmaceutical composition comprises a plurality of FMRP polypeptide or FMRP polynucleotide-nanoparticle formulations. Each method represents a separate embodiment of the present invention.

In some embodiments, one or more FMRP polypeptides or polynucleotides encoding a FMRP polypeptide is delivered to a target tissue in vivo in a vesicle, e.g., a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, where the composition comprises more than one FMRP polypeptide or polynucleotide, each FMRP polypeptide or polynucleotide can be formulated as its own liposome formulation, and a pharmaceutical composition can comprise a plurality of FMRP polypeptide or polynucleotide-liposome formulations.

In some embodiments, compositions comprising at least one FMRP polypeptide or polynucleotide for delivery to a target tissue in vivo as disclosed herein can be, in another embodiment, administered to a subject by any method known to a person skilled in the art, such as parenterally, intramuscularly, intra-dermally, subcutaneously, intra-peritonealy, or intra-ventricularly. In another embodiment of methods and compositions of the present invention, compositions comprising at least one FMRP polypeptide or polynucleotide for delivery to a target tissue in vivo as disclosed herein are formulated in a form suitable for injection, i.e., as a liquid preparation. Suitable liquid formulations include solutions, suspensions, dispersions, emulsions, oils and the like. In another embodiment of the present invention, the active ingredient is formulated in a capsule, e.g., a slow release capsule.

In other embodiments, the pharmaceutical compositions comprising at least one FMRP polypeptide or polynucleotide for delivery to a target tissue in vivo as disclosed herein can be administered by intra-arterial, or intramuscular injection of a liquid preparation. Suitable liquid formulations include solutions, suspensions, dispersions, emulsions, oils and the like. In another embodiment, the pharmaceutical compositions comprising at least one FMRP polypeptide or polynucleotide as disclosed herein can be administered intra-arterially and are thus formulated in a form suitable for intra-arterial administration.

In another embodiment, a pharmaceutical composition comprising at least one FMRP polypeptide or polynucleotide for delivery to a target tissue in vivo as disclosed herein can be administered topically to body surfaces and are thus formulated in a form suitable for topical administration. Suitable topical formulations include gels, ointments, creams, lotions, drops and the like. For topical administration, the compositions or their physiologically tolerated derivatives are prepared and applied as solutions, suspensions, or emulsions in a physiologically acceptable diluent with or without a pharmaceutical carrier.

As used herein “pharmaceutically acceptable carriers, excipients, or diluents” are well known to those skilled in the art. The carrier, excipient, or diluent may be may, in various embodiments, a solid carrier or diluent for solid formulations, a liquid carrier or diluent for liquid formulations, or mixtures thereof. In another embodiment, solid carriers/diluents include, but are not limited to, a gum, a starch (e.g. corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. In other embodiments, pharmaceutically acceptable carriers for liquid formulations may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In another embodiment, a compositions for delivery of a FMRP polypeptide or polynucleotide to a target tissue in vivo further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., TWEEN 20, TWEEN 80, Pluronic F68, bile acid salts), protease inhibitors, surfactants (e.g., sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants. Each of the above excipients represents a separate embodiment of the present invention.

In another embodiment, a pharmaceutical composition for delivery of a FMRP polypeptide or polynucleotide to a target tissue in vivo can comprise a FMRP polypeptide or polynucleotide in a controlled-release composition, i.e., a composition in which the compound is released over a period of time after administration. Controlled- or sustained-release compositions include formulation in lipophilic depots (e.g., fatty acids, waxes, oils). In another embodiment, a composition for delivery of a FMRP polypeptide or polynucleotide to a target tissue in vivo is an immediate-release composition, i.e., a composition in which the entire compound is released immediately after administration.

In another embodiment, for delivery of a FMRP polypeptide or polynucleotide to a target tissue in vivo, one can modify a FMRP polypeptide or polynucleotide of the present invention by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Davis, S. et al., Clin. Exp Immunol, 46(3):649-652 (1981); and Katre et al., Proc. Natl Acad. Sci 84(6): 1487-1491 (1987). Such modifications also increase, in another embodiment, the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In another embodiment, a composition for delivery of a FMRP polypeptide or polynucleotide to a target tissue in vivo is formulated to include a neutralized pharmaceutically acceptable salt form. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. Each of the above additives, excipients, formulations and methods of administration represents a separate embodiment of the present invention.

In some embodiments of the aspects described herein, involving in vivo administration of FMRP polypeptide or polynucleotide or compositions thereof to a target tissue in vivo, are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

A composition comprising at least one FMRP polypeptide or polynucleotide as disclosed herein can be formulated into any of many possible administration forms, including a sustained release form. The compositions can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

A composition comprising at least one FMRP polypeptide or polynucleotide as disclosed herein can be prepared and formulated as emulsions for the delivery of polypeptides or polynucleotides. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain further components in addition to the dispersed phases, and the active agent (i.e., FMRP polypeptide or polynucleotide), which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

As noted above, liposomes can optionally be prepared to contain surface groups to facilitate delivery of liposomes and their contents to specific cell populations. For example, a liposome can comprise a surface groups such as antibodies or antibody fragments, small effector molecules for interacting with cell-surface receptors, antigens, and other like compounds. Surface groups can be incorporated into the liposome by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes can include an RNA molecule. U.S. Pat. No. 5,665,710 (Rahman et al.) describes certain methods of encapsulating oligodeoxynucleotides in liposomes. WO 97/04787 (Love et al.) discloses liposomes comprising RNAi molecules targeted to the raf gene. In addition, methods for preparing a liposome composition comprising a nucleic acid can be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337. Each of these approaches can provide delivery of a FMRP polynucleotide as described herein to a cell.

In some embodiments of the aspects described herein, a composition comprising at least one FMRP polypeptide or polynucleotide for in vivo protein expression in a target tissue as disclosed herein can be encapsulated in a nanoparticle. Methods for nanoparticle packaging are well known in the art, and are described, for example, in Bose S, et al., J. Virol. 78:8146 (2004); Dong, Y et al., Biomaterials 26:6068 (2005); Lobenberg R. et al., J Drug Target 5:171.1998); Sakuma S R et al., Int J Pharm 177:161, (1999); Virovic L et al., Expert Opin Drug Deliv 2:707 (2005); and Zimmermann E et al., Eur J Pharm Biopharm 52:203 (2001), the contents of which are herein incorporated by reference in their entireties.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: High-Confidence Fmrp RNA Targets in Hpscs and Neurons Defined by eCLIP-Seq

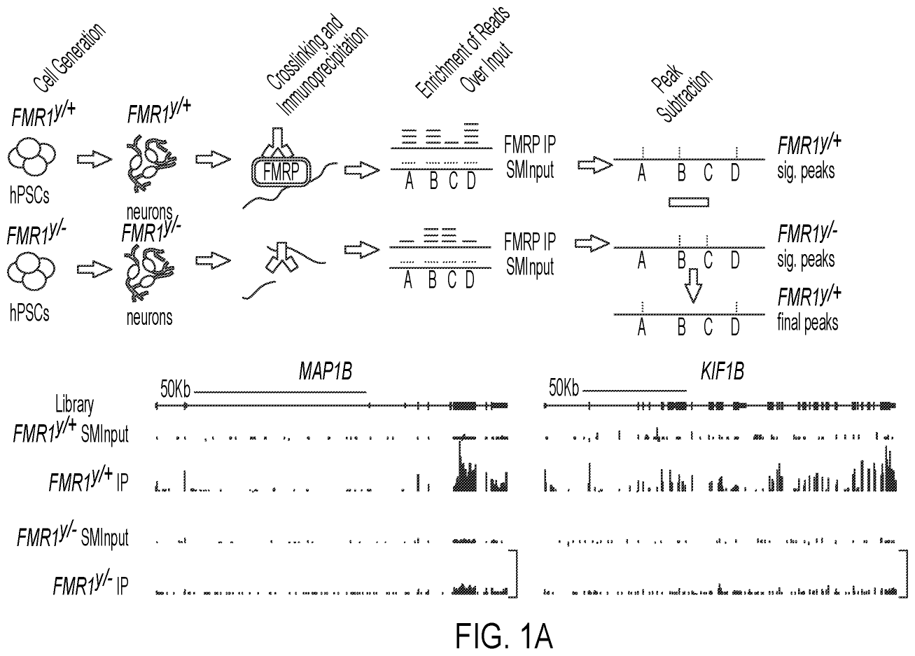

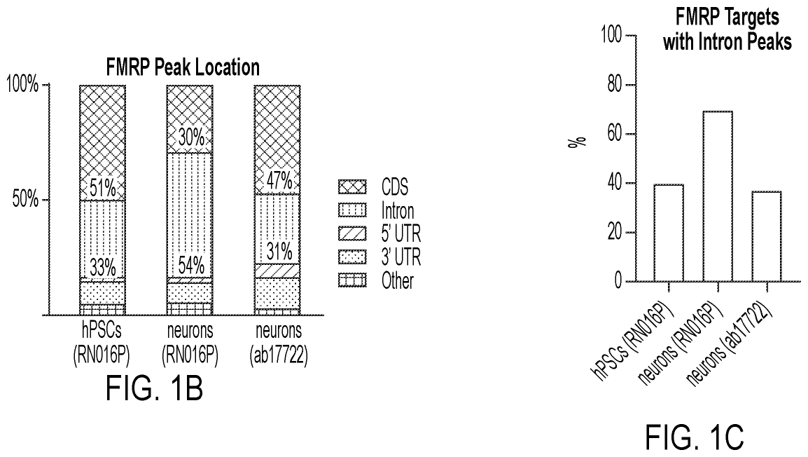

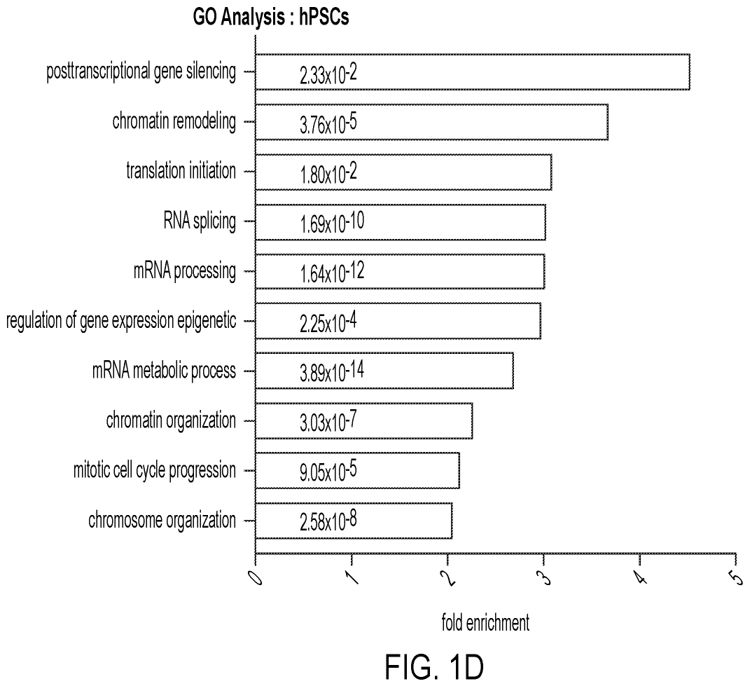

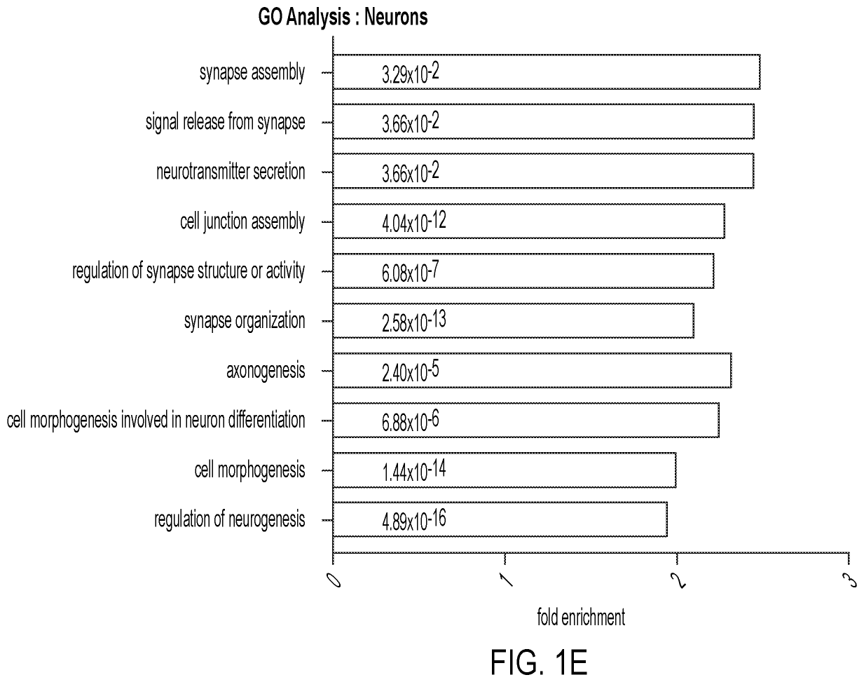

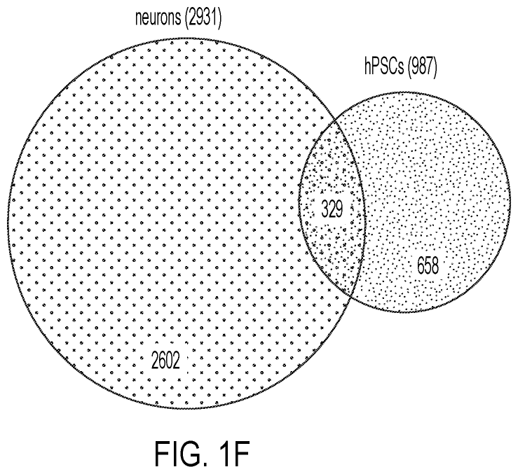

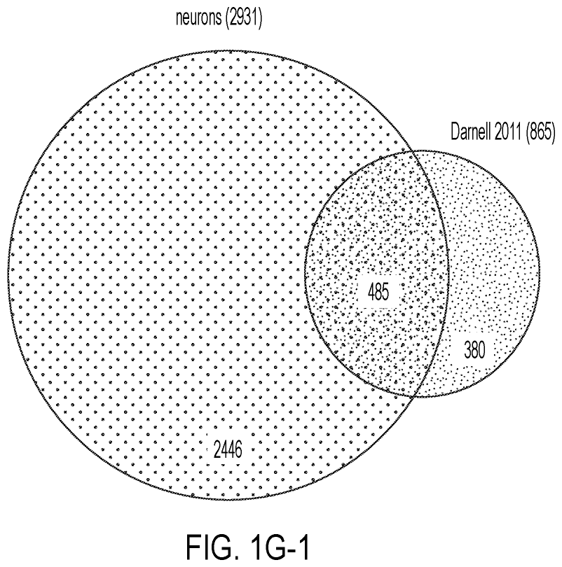

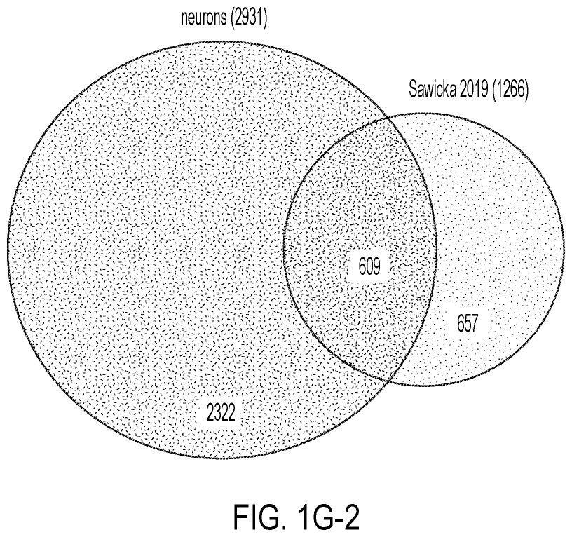

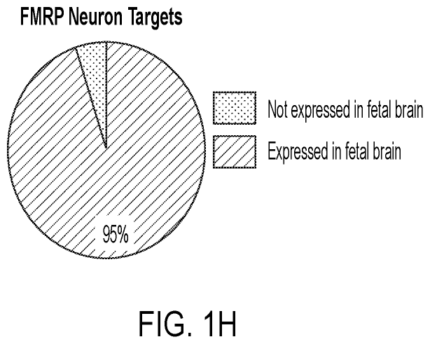

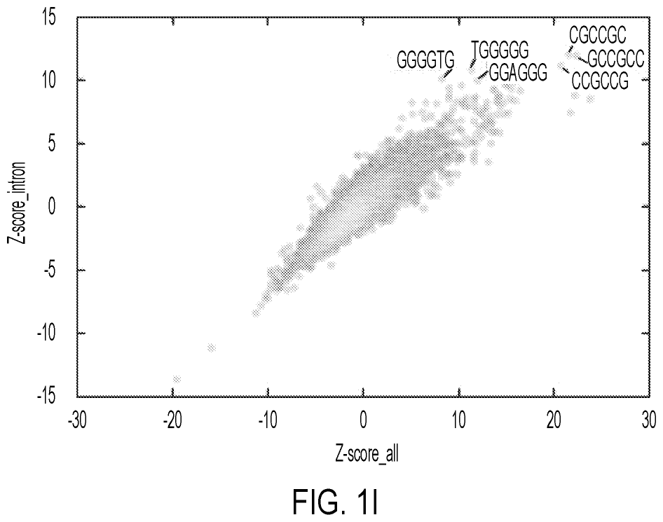

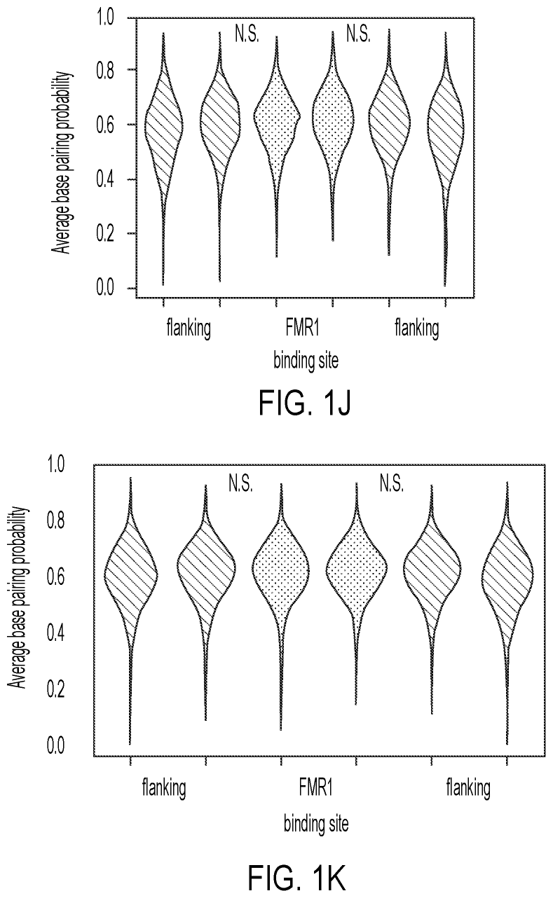

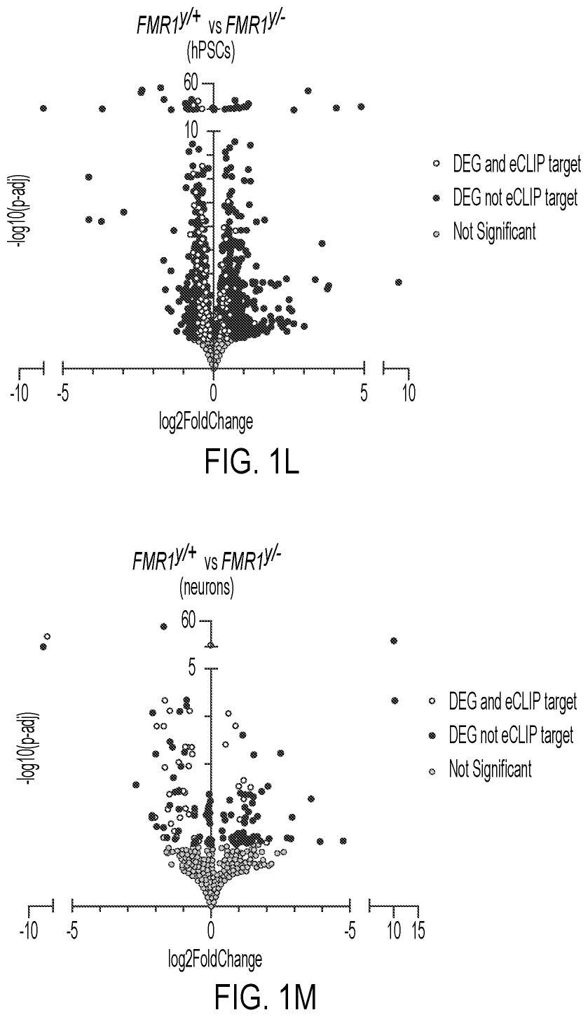

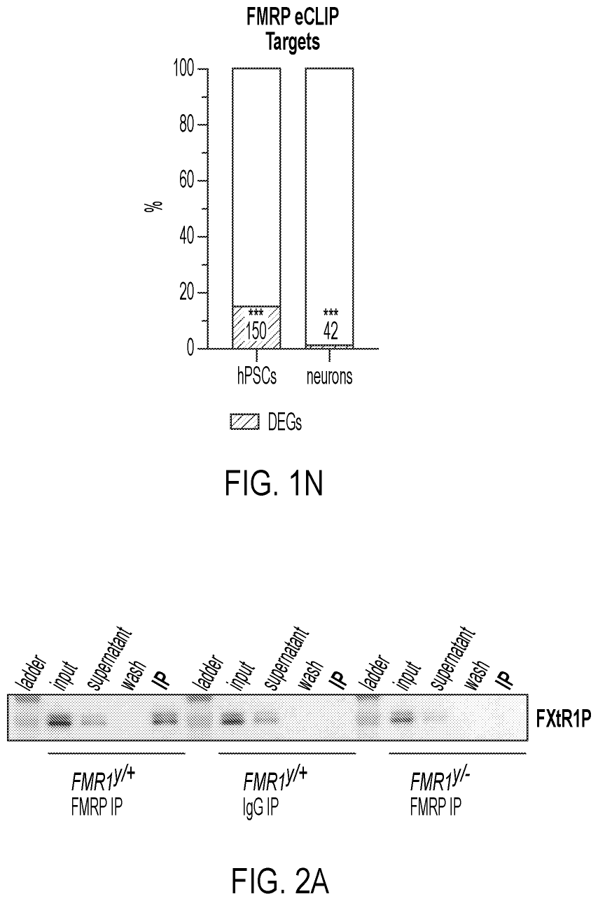

To define the RNA targets of FMRP in both hPSCs and excitatory cortical neurons, eCLIP-seq was used with two independent anti-FMRP antibodies (RN016P (MBL International) and ab17722 (Abcam)) (FIG. 1A, Table 1), both of which have previously been used for FMRP eCLIP-seq in non-neuronal cell types. For all eCLIP-seq experiments, significant peaks were identified by comparing peak read density between eCLIP and a paired size-matched input (SMInput) (FIG. 1A), excluding PCR duplicates and considering only uniquely mapped reads as previously described (Van Nostrand, E. L. et al., Nature methods 13, 508-14 (2016). A stringent peak threshold of at least 8-fold enrichment in immunoprecipitation over the SMInput with p-values <10⁻³ was used. To reduce potential non-specific binding events, the same eCLIP-seq experiments were performed utilizing isogenic FMR1^{y/-} hPSCs and neurons and significant peaks detected in each dataset were subtracted from the corresponding FMR1^{y/+} datasets (FIG. 1A, Table 1). Importantly, FMR1^{y/-} peak subtraction eliminated 18-33% of FMRP RNA targets from each FMR1^{y/+} dataset (Table 1), focusing the analyses on targets with highest specificity.

TABLE 1

| | | Significant | | | |
|---|---|---|---|---|---|
| Cell Type | Antibody | Peaks | Genes | Cell Input | % Subtracted |
| hPSCs | RN016P | 1688 | 987 | 1.5E+07 | 30.2 |
| Neurons | RN016P | 363 | 93 | 1.5E+07 | 33.1 |
| Neurons | Ab17722 | 6634 | 2889 | 7.0E+07 | 17.8 |

All subsequent analyses were performed using significant peaks and gene targets that remained following FMR1^{y/-} peak subtraction (Table 1). Of note, the FMRP antibody RN016P (MBL Intl.) neuron datasets were generated using a smaller number of neurons as input compared with the FMRP antibody ab17722 (Abcam) neuron datasets, and thus yielded fewer significant peaks and genes.

Across all FMRP eCLIP-seq datasets, a consistent pattern of peak distributions was observed, with the majority of peaks located in CDS and introns and the remaining peaks distributed across other gene elements (FIG. 1B). Intronic peaks were found in roughly 40% of all FMRP targets (FIG. 1C), consistent with FMRP binding these targets as unspliced pre-mRNAs. Several studies previously identified FMRP RNA targets in mouse brain but did not report the presence of intron binding events, which may be due in part to polysome enrichment employed in two of these studies. Consistent with the datasets described herein, four separate studies of FMRP RNA targets in human cells or tissue including HEK293T cells, K562 cells, and human postmortem brain all detected FMRP intron binding events.

Gene Ontology (GO) analysis revealed FMRP targets that are themselves involved in different aspects of RNA and DNA regulation in hPSCs, including chromatin remodeling and RNA splicing (FIG. 1D, Table 2). In neurons, not surprisingly, GO analysis pointed to targets related to neuronal development (FIG. 1E, Table 2). FMRP targets in hPSCs and neurons were minimally overlapping (FIG. 1F), indicative of largely distinct functions of FMRP in pluripotent stem cells versus post-mitotic neurons. Interestingly, while 56.07% of FMRP targets previously identified in mouse brain (Darnell, J. C. et al., Cell, 146, 247-261 (2011)) were FMRP targets inhuman neurons, representing a 2.93-fold enrichment over expected (p=3.92×10⁻¹³⁸), 83.5% of human neuron targets did not overlap with those previously identified in mouse brain′ (FIG. 1F), suggesting that existing knowledge of FMRP target biology remains incomplete. Notably, the vast majority (95%) of FMRP targets identified in human neuron in vitro were also detectably expressed in human fetal brain tissue (FIG. 1G), supporting the in vivo relevance of the neuronal transcriptomes. Collectively, these data define high-confidence FMRP RNA targets for the first time in hPSCs and neurons and identify FMRP binding events on pre-mRNA targets in both the hPSC and neuron cell types.

TABLE 2
GO Analysis
Analysis Type: PANTHER Overrepresentation Test (Released 2019 Jul. 11)
Annotation Version and Release Date: GO Ontology database Released 2019 Oct. 8
Analyzed List: upload_1 (Homo sapiens)
Reference List: stem cell universe.csv (Homo sapiens)
Test Type: FISHER
Correction: BONFERRONI
Bonferroni count: 7847

| | stem cell | | | | | |
|---|---|---|---|---|---|---|
| | universe.csv - | | | | upload_1 | |
| | REFLIST | upload_1 | upload_1 | upload_1 | (fold | upload_1 |
| GO biological process complete | (14063) | (858) | (expected) | (over/under) | Enrichment) | (P-value) |
| posttranscriptional gene silencing by RNA | 57 | 16 | 3.48 | + | 4.6 | 2.33E−02 |
| (GO:0035194) | | | | | | |
| posttranscriptional gene silencing | 58 | 16 | 3.54 | + | 4.52 | 2.82E−02 |
| (GO:0016441) | | | | | | |
| chromatin remodeling (GO:0006338) | 143 | 32 | 8.72 | + | 3.67 | 3.76E−05 |
| regulation of mRNA processing | 131 | 28 | 7.99 | + | 3.5 | 7.90E−04 |
| (GO:0050684) | | | | | | |
| regulation of RNA splicing (GO:0043484) | 128 | 25 | 7.81 | + | 3.2 | 1.60E−02 |
| mRNA transport (GO:0051028) | 144 | 28 | 8.79 | + | 3.19 | 4.27E−03 |
| gene silencing (GO:0016458) | 131 | 25 | 7.99 | + | 3.13 | 2.29E−02 |
| translational initiation (GO:0006413) | 138 | 26 | 8.42 | + | 3.09 | 1.80E−02 |
| RNA transport (GO:0050658) | 178 | 33 | 10.86 | + | 3.04 | 1.12E−03 |
| nucleic acid transport (GO:0050657) | 178 | 33 | 10.86 | + | 3.04 | 1.12E−03 |
| RNA splicing via transesterification reactions | 281 | 52 | 17.14 | + | 3.03 | 3.00E−07 |
| (GO:0000375) | | | | | | |
| RNA splicing (GO:0008380) | 373 | 69 | 22.76 | + | 3.03 | 1.69E−10 |
| mRNA processing (GO:0006397) | 435 | 80 | 26.54 | + | 3.01 | 1.64E−12 |
| mRNA splicing via spliceosome | 278 | 51 | 16.96 | + | 3.01 | 6.11E−07 |
| (GO:0000398) | | | | | | |
| RNA splicing via transesterification reactions | 278 | 51 | 16.96 | + | 3.01 | 6.11E−07 |
| with bulged adenosine as nucleophile | | | | | | |
| (GO:0000377) | | | | | | |
| establishment of RNA localization | 180 | 33 | 10.98 | + | 3 | 1.41E−03 |
| (GO:0051236) | | | | | | |
| regulation of gene expression epigenetic | 210 | 38 | 12.81 | + | 2.97 | 2.25E−04 |
| (GO:0040029) | | | | | | |
| mitotic prometaphase (GO:0000236) | 167 | 30 | 10.19 | + | 2.94 | 7.35E−03 |
| RNA localization (GO:0006403) | 200 | 35 | 12.2 | + | 2.87 | 1.61E−03 |
| cytoskeleton-dependent intracellular transport | 166 | 28 | 10.13 | + | 2.76 | 4.88E−02 |
| (GO:0030705) | | | | | | |
| regulation of mRNA metabolic process | 298 | 49 | 18.18 | + | 2.7 | 4.39E−05 |
| (GO:1903311) | | | | | | |
| mRNA metabolic process (GO:0016071) | 640 | 105 | 39.05 | + | 2.69 | 3.89E−14 |
| rhythmic process (GO:0048511) | 217 | 35 | 13.24 | + | 2.64 | 1.10E−02 |
| nucleobase-containing compound transport | 217 | 35 | 13.24 | + | 2.64 | 1.10E−02 |
| (GO:0015931) | | | | | | |
| positive regulation of cell cycle process | 244 | 37 | 14.89 | + | 2.49 | 2.34E−02 |
| (GO:0090068) | | | | | | |
| regulation of chromosome organization | 321 | 46 | 19.58 | + | 2.35 | 4.59E−03 |
| (GO:0033044) | | | | | | |
| RNA processing (GO:0006396) | 806 | 113 | 49.17 | + | 2.3 | 5.16E−11 |
| chromatin organization (GO:0006325) | 616 | 85 | 37.58 | + | 2.26 | 3.03E−07 |
| covalent chromatin modification | 341 | 46 | 20.8 | + | 2.21 | 2.84E−02 |
| (GO:0016569) | | | | | | |
| regulation of mitotic cell cycle phase | 390 | 51 | 23.79 | + | 2.14 | 1.52E−02 |
| transition (GO:1901990) | | | | | | |
| mitotic cell cycle process (GO:1903047) | 573 | 74 | 34.96 | + | 2.12 | 9.05E−05 |
| posttranscriptional regulation of gene | 487 | 62 | 29.71 | + | 2.09 | 2.37E−03 |
| expression (GO:0010608) | | | | | | |
| chromosome organization (GO:0051276) | 940 | 117 | 57.35 | + | 2.04 | 2.58E−08 |
| regulation of cell cycle process (GO:0010564) | 670 | 82 | 40.88 | + | 2.01 | 1.42E−04 |
| mitotic cell cycle (GO:0000278) | 647 | 79 | 39.47 | + | 2 | 3.44E−04 |
| RNA metabolic process (GO:0016070) | 1483 | 177 | 90.48 | + | 1.96 | 5.68E−13 |
| negative regulation of cellular macromolecule | 1118 | 132 | 68.21 | + | 1.94 | 2.87E−08 |
| biosynthetic process (GO:2000113) | | | | | | |
| symbiotic process (GO:0044403) | 682 | 80 | 41.61 | + | 1.92 | 1.14E−03 |
| negative regulation of macromolecule | 1181 | 138 | 72.05 | + | 1.92 | 1.61E−08 |
| biosynthetic process (GO:0010558) | | | | | | |
| viral process (GO:0016032) | 626 | 73 | 38.19 | + | 1.91 | 5.22E−03 |
| negative regulation of cellular biosynthetic | 1228 | 142 | 74.92 | + | 1.9 | 1.59E−08 |
| process (GO:0031327) | | | | | | |
| negative regulation of nucleobase-containing | 1152 | 133 | 70.28 | + | 1.89 | 8.84E−08 |
| compound metabolic process (GO:0045934) | | | | | | |
| nucleic acid metabolic process (GO:0090304) | 2026 | 233 | 123.61 | + | 1.88 | 6.52E−17 |
| regulation of mitotic cell cycle (GO:0007346) | 575 | 66 | 35.08 | + | 1.88 | 2.73E−02 |
| microtubule-based process (GO:0007017) | 576 | 66 | 35.14 | + | 1.88 | 2.82E−02 |
| negative regulation of RNA metabolic process | 1061 | 121 | 64.73 | + | 1.87 | 1.99E−06 |
| (GO:0051253) | | | | | | |
| negative regulation of biosynthetic process | 1251 | 142 | 76.32 | + | 1.86 | 5.73E−08 |
| (GO:0009890) | | | | | | |
| interspecies interaction between organisms | 705 | 80 | 43.01 | + | 1.86 | 4.08E−03 |
| (GO:0044419) | | | | | | |
| negative regulation of gene expression | 1403 | 156 | 85.6 | + | 1.82 | 1.57E−08 |
| (GO:0010629) | | | | | | |
| negative regulation of nucleic acid-templated | 981 | 109 | 59.85 | + | 1.82 | 7.53E−05 |
| transcription (GO:1903507) | | | | | | |
| negative regulation of RNA biosynthetic | 982 | 109 | 59.91 | + | 1.82 | 7.65E−05 |
| process (GO:1902679) | | | | | | |
| gene expression (GO:0010467) | 1784 | 198 | 108.84 | + | 1.82 | 5.45E−12 |
| negative regulation of transcription DNA- | 938 | 103 | 57.23 | + | 1.8 | 2.95E−04 |
| templated (GO:0045892) | | | | | | |
| cellular response to DNA damage stimulus | 730 | 80 | 44.54 | + | 1.8 | 1.38E−02 |
| (GO:0006974) | | | | | | |
| cytoskeleton organization (GO:0007010) | 924 | 100 | 56.37 | + | 1.77 | 1.09E−03 |
| regulation of cell cycle (GO:0051726) | 1052 | 111 | 64.18 | + | 1.73 | 6.55E−04 |
| positive regulation of nucleobase-containing | 1466 | 153 | 89.44 | + | 1.71 | 2.00E−06 |
| compound metabolic process (GO:0045935) | | | | | | |
| negative regulation of nitrogen compound | 1871 | 194 | 114.15 | + | 1.7 | 7.16E−09 |
| metabolic process (GO:0051172) | | | | | | |
| regulation of organelle organization | 1139 | 118 | 69.49 | + | 1.7 | 6.47E−04 |
| (GO:0033043) | | | | | | |
| nucleobase-containing compound metabolic | 2459 | 254 | 150.03 | + | 1.69 | 2.96E−13 |
| process (GO:0006139) | | | | | | |
| positive regulation of gene expression | 1538 | 158 | 93.84 | + | 1.68 | 3.11E−06 |
| (GO:0010628) | | | | | | |
| negative regulation of macromolecule | 2132 | 219 | 130.08 | + | 1.68 | 2.40E−10 |
| metabolic process (GO:0010605) | | | | | | |
| positive regulation of RNA metabolic process | 1324 | 135 | 80.78 | + | 1.67 | 1.17E−04 |
| (GO:0051254) | | | | | | |
| negative regulation of cellular metabolic | 2035 | 207 | 124.16 | + | 1.67 | 4.81E−09 |
| process (GO:0031324) | | | | | | |
| negative regulation of metabolic process | 2328 | 235 | 142.03 | + | 1.65 | 1.01E−10 |
| (GO:0009892) | | | | | | |
| positive regulation of macromolecule | 1469 | 148 | 89.63 | + | 1.65 | 4.80E−05 |
| biosynthetic process (GO:0010557) | | | | | | |
| positive regulation of nucleic acid-templated | 1246 | 125 | 76.02 | + | 1.64 | 1.08E−03 |
| transcription (GO:1903508) | | | | | | |
| positive regulation of RNA biosynthetic | 1247 | 125 | 76.08 | + | 1.64 | 1.10E−03 |
| process (GO:1902680) | | | | | | |
| heterocycle metabolic process (GO:0046483) | 2581 | 258 | 157.47 | + | 1.64 | 7.14E−12 |
| cellular aromatic compound metabolic process | 2603 | 257 | 158.81 | + | 1.62 | 3.68E−11 |
| (GO:0006725) | | | | | | |
| positive regulation of cellular biosynthetic | 1535 | 150 | 93.65 | + | 1.6 | 2.42E−04 |
| process (GO:0031328) | | | | | | |
| positive regulation of biosynthetic process | 1558 | 152 | 95.06 | + | 1.6 | 2.16E−04 |
| (GO:0009891) | | | | | | |
| positive regulation of transcription DNA- | 1170 | 114 | 71.38 | + | 1.6 | 1.76E−02 |
| templated (GO:0045893) | | | | | | |
| cell cycle (GO:0007049) | 1191 | 116 | 72.66 | + | 1.6 | 1.54E−02 |
| protein-containing complex assembly | 1245 | 121 | 75.96 | + | 1.59 | 9.84E−03 |
| (GO:0065003) | | | | | | |
| organic cyclic compound metabolic process | 2766 | 267 | 168.76 | + | 1.58 | 1.06E−10 |
| (GO:1901360) | | | | | | |
| regulation of transcription by RNA | 1727 | 164 | 105.37 | + | 1.56 | 2.56E−04 |
| polymerase II (GO:0006357) | | | | | | |
| regulation of RNA metabolic process | 2965 | 280 | 180.9 | + | 1.55 | 2.36E−10 |
| (GO:0051252) | | | | | | |
| positive regulation of macromolecule | 2627 | 248 | 160.28 | + | 1.55 | 1.99E−08 |
| metabolic process (GO:0010604) | | | | | | |
| cellular nitrogen compound metabolic process | 2979 | 280 | 181.75 | + | 1.54 | 3.74E−10 |
| (GO:0034641) | | | | | | |
| regulation of nucleobase-containing | 3195 | 299 | 194.93 | + | 1.53 | 5.75E−11 |
| compound metabolic process (GO:0019219) | | | | | | |
| regulation of gene expression (GO:0010468) | 3541 | 329 | 216.04 | + | 1.52 | 1.82E−12 |
| organelle organization (GO:0006996) | 2993 | 276 | 182.61 | + | 1.51 | 7.09E−09 |
| positive regulation of metabolic process | 2842 | 262 | 173.39 | + | 1.51 | 3.39E−08 |
| (GO:0009893) | | | | | | |
| regulation of macromolecule biosynthetic | 3191 | 291 | 194.69 | + | 1.49 | 3.62E−09 |
| process (GO:0010556) | | | | | | |
| regulation of cellular macromolecule | 3094 | 282 | 188.77 | + | 1.49 | 1.00E−08 |
| biosynthetic process (GO:2000112) | | | | | | |
| regulation of cellular biosynthetic process | 3299 | 296 | 201.28 | + | 1.47 | 1.23E−08 |
| (GO:0031326) | | | | | | |
| positive regulation of nitrogen compound | 2488 | 223 | 151.8 | + | 1.47 | 3.58E−05 |
| metabolic process (GO:0051173) | | | | | | |
| positive regulation of cellular metabolic | 2605 | 232 | 158.93 | + | 1.46 | 2.89E−05 |
| process (GO:0031325) | | | | | | |
| regulation of biosynthetic process | 3364 | 299 | 205.24 | + | 1.46 | 3.02E−08 |
| (GO:0009889) | | | | | | |
| regulation of cellular component organization | 2108 | 187 | 128.61 | + | 1.45 | 2.20E−03 |
| (GO:0051128) | | | | | | |
| regulation of macromolecule metabolic | 4862 | 431 | 296.64 | + | 1.45 | 6.53E−16 |
| process (GO:0060255) | | | | | | |
| multi-organism process (GO:0051704) | 1880 | 165 | 114.7 | + | 1.44 | 3.02E−02 |
| nervous system development (GO:0007399) | 1871 | 164 | 114.15 | + | 1.44 | 3.69E−02 |
| regulation of nucleic acid-templated | 2742 | 240 | 167.29 | + | 1.43 | 5.69E−05 |
| transcription (GO:1903506) | | | | | | |
| regulation of RNA biosynthetic process | 2746 | 240 | 167.54 | + | 1.43 | 5.90E−05 |
| (GO:2001141) | | | | | | |
| regulation of nitrogen compound metabolic | 4589 | 401 | 279.98 | + | 1.43 | 8.87E−13 |
| process (GO:0051171) | | | | | | |
| negative regulation of biological process | 4184 | 361 | 255.27 | + | 1.41 | 9.82E−10 |
| (GO:0048519) | | | | | | |
| negative regulation of cellular process | 3727 | 321 | 227.39 | + | 1.41 | 1.12E−07 |
| (GO:0048523) | | | | | | |
| regulation of cellular metabolic process | 4913 | 423 | 299.75 | + | 1.41 | 5.62E−13 |
| (GO:0031323) | | | | | | |
| regulation of primary metabolic process | 4740 | 407 | 289.19 | + | 1.41 | 7.25E−12 |
| (GO:0080090) | | | | | | |
| regulation of transcription DNA-templated | 2692 | 231 | 164.24 | + | 1.41 | 5.92E−04 |
| (GO:0006355) | | | | | | |
| regulation of metabolic process (GO:0019222) | 5291 | 454 | 322.81 | + | 1.41 | 1.05E−14 |
| cellular localization (GO:0051641) | 2128 | 182 | 129.83 | + | 1.4 | 3.57E−02 |
| animal organ development (GO:0048513) | 2229 | 190 | 135.99 | + | 1.4 | 2.69E−02 |
| regulation of multicellular organismal process | 2341 | 198 | 142.83 | + | 1.39 | 2.12E−02 |
| (GO:0051239) | | | | | | |
| macromolecule metabolic process | 5167 | 435 | 315.24 | + | 1.38 | 5.59E−12 |
| (GO:0043170) | | | | | | |
| cell differentiation (GO:0030154) | 2621 | 219 | 159.91 | + | 1.37 | 1.20E−02 |
| system development (GO:0048731) | 3224 | 269 | 196.7 | + | 1.37 | 3.76E−04 |
| cellular developmental process (GO:0048869) | 2708 | 222 | 165.22 | + | 1.34 | 3.80E−02 |
| cellular macromolecule metabolic process | 4346 | 355 | 265.15 | + | 1.34 | 2.88E−06 |
| (GO:0044260) | | | | | | |
| cellular component organization | 4664 | 377 | 284.56 | + | 1.32 | 1.76E−06 |
| (GO:0016043) | | | | | | |
| positive regulation of cellular process | 4204 | 339 | 256.49 | + | 1.32 | 5.56E−05 |
| (GO:0048522) | | | | | | |
| positive regulation of biological process | 4682 | 377 | 285.65 | + | 1.32 | 2.97E−06 |
| (GO:0048518) | | | | | | |
| cellular component organization or biogenesis | 4845 | 390 | 295.6 | + | 1.32 | 9.72E−07 |
| (GO:0071840) | | | | | | |
| multicellular organism development | 3670 | 293 | 223.91 | + | 1.31 | 3.18E−03 |
| (GO:0007275) | | | | | | |
| cellular protein metabolic process | 3181 | 253 | 194.08 | + | 1.3 | 4.84E−02 |
| (GO:0044267) | | | | | | |
| anatomical structure development | 3949 | 310 | 240.93 | + | 1.29 | 5.65E−03 |
| (GO:0048856) | | | | | | |
| nitrogen compound metabolic process | 5822 | 454 | 355.21 | + | 1.28 | 3.78E−07 |
| (GO:0006807) | | | | | | |
| developmental process (GO:0032502) | 4288 | 332 | 261.62 | + | 1.27 | 5.71E−03 |
| primary metabolic process (GO:0044238) | 6154 | 471 | 375.46 | + | 1.25 | 2.04E−06 |
| multicellular organismal process | 4652 | 353 | 283.82 | + | 1.24 | 1.40E−02 |
| (GO:0032501) | | | | | | |
| cellular metabolic process (GO:0044237) | 6384 | 484 | 389.5 | + | 1.24 | 2.96E−06 |
| organic substance metabolic process | 6420 | 484 | 391.69 | + | 1.24 | 7.44E−06 |
| (GO:0071704) | | | | | | |
| regulation of cellular process (GO:0050794) | 7995 | 596 | 487.78 | + | 1.22 | 1.43E−09 |
| regulation of biological process (GO:0050789) | 8480 | 630 | 517.37 | + | 1.22 | 3.90E−11 |
| metabolic process (GO:0008152) | 6858 | 497 | 418.41 | + | 1.19 | 1.45E−03 |
| biological regulation (GO:0065007) | 8970 | 649 | 547.27 | + | 1.19 | 3.77E−09 |
| cellular process (GO:0009987) | 10694 | 728 | 652.45 | + | 1.12 | 7.00E−06 |
| biological process (GO:0008150) | 12719 | 814 | 776 | + | 1.05 | 3.02E−02 |
| Unclassified (UNCLASSIFIED) | 1344 | 44 | 82 | − | 0.54 | 0.00E+00 |

### Example 2: A Majority of FMRP Binding Events in Neurons are Mediated by the Key Co-Factor FXR1P

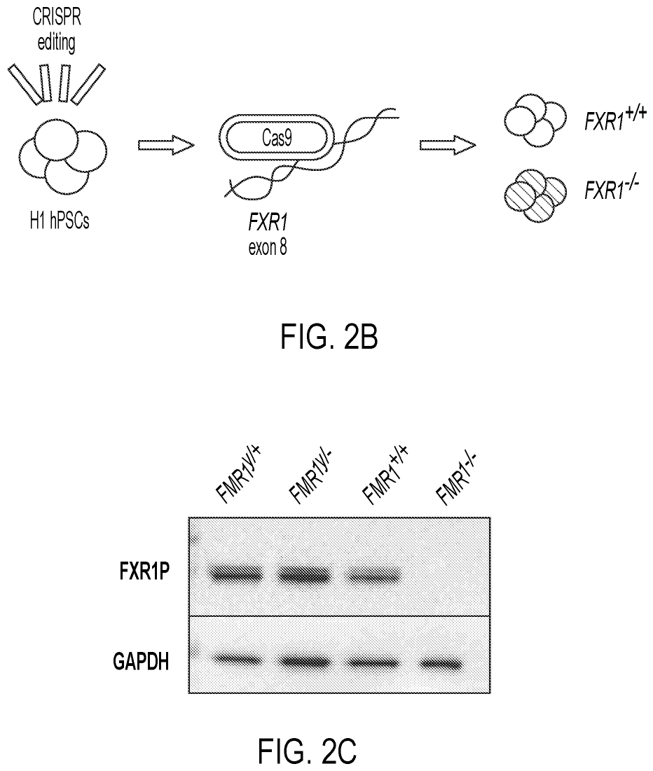

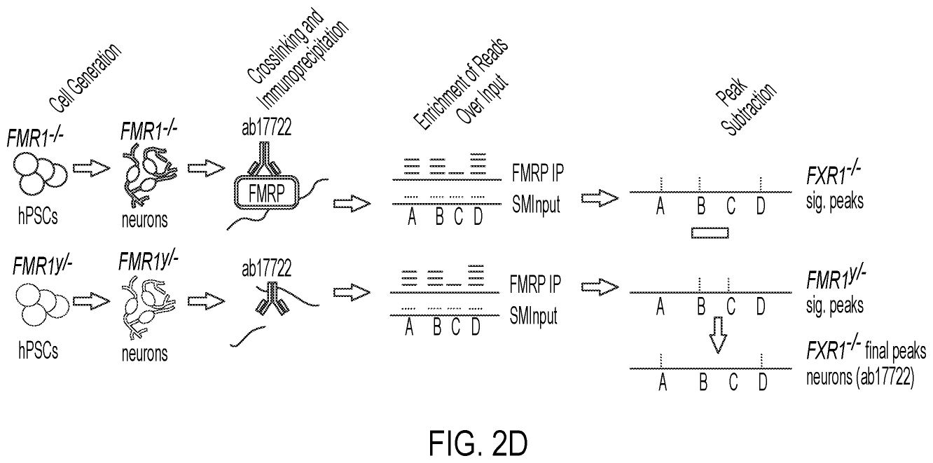

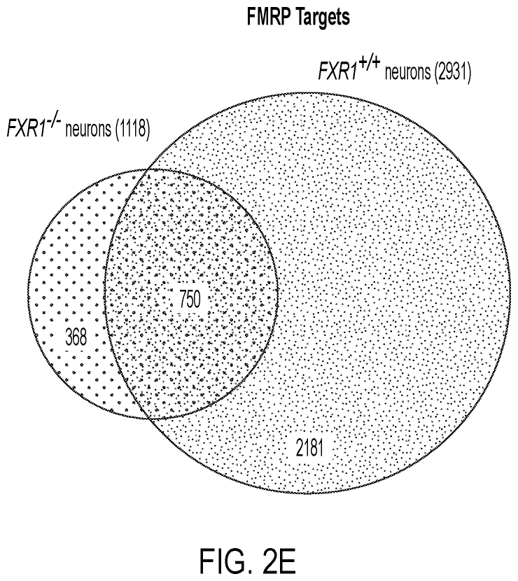

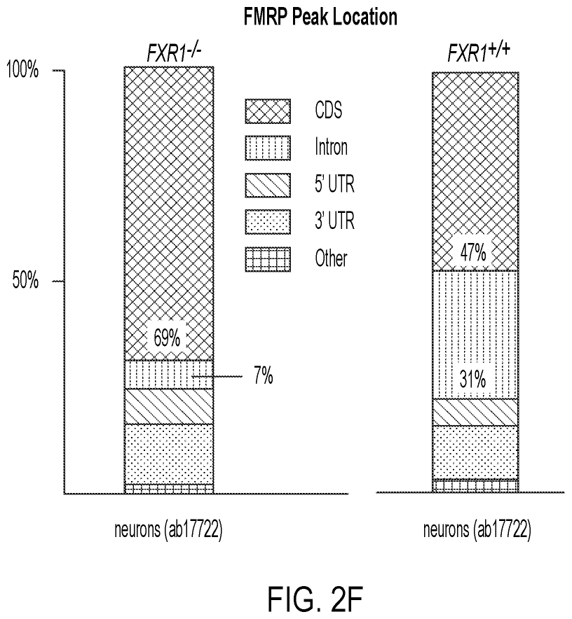

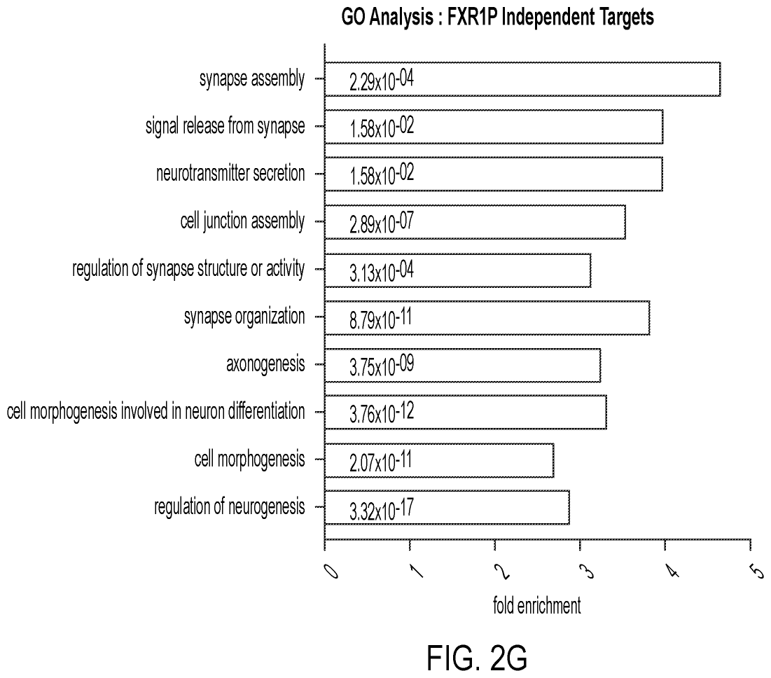

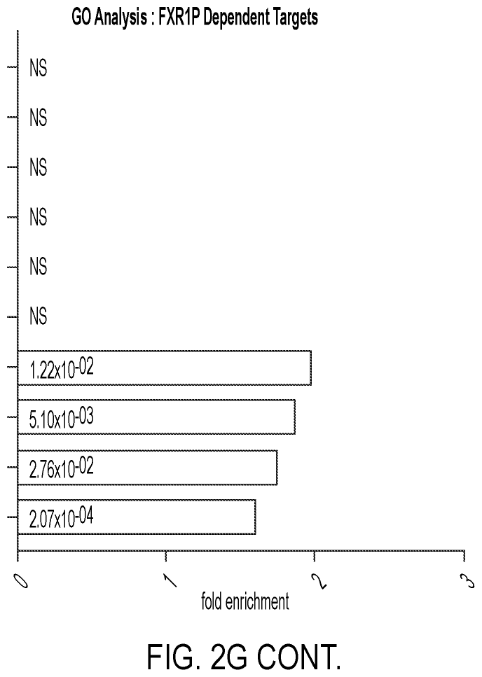

The role of FMRP's autosomal paralog FXR1P, encoded by the Fragile X mental retardation syndrome-related 1 (FXR1) gene was investigated. FMRP and FXR1P contain highly similar domains, are co-expressed in neurons, can form homomers or heteromers and are reported to act in synergy as opposed to having redundant functions. The detection of intron binding events in the datasets described herein (FIG. 1B) indicates that FMRP is capable of binding pre-mRNAs in addition to mature, fully spliced mRNAs. A majority of intronic peaks were distally located (>500 nucleotides from the closest intron-exon junction) and an RNA sequence motif or secondary structure uniquely associated with intron binding was not detected (FIGS. 1I, 1J, 1K), suggesting that other features drive FMRP target recognition in these datasets. While little is known about the role of FMRP in the regulation of pre-mRNA targets, one of the few known nuclear co-factors of FMRP is its autosomal paralog FXR1P⁵², encoded by the Fragile X mental retardation syndrome-related 1 (FXR1) gene. Given their propensity to heterodimerize (FIG. 2A), it was contemplated whether FMRP binding events, including pre-mRNA binding events, were dependent or independent of its co-factor FXR1P. To this end, an FXR1^{-/-} hPSC line was generated using CRISPR-Cas9 (FIG. 2B), isogenic with the FMR1^{y/+} and FMR1^{y/-} hPSC lines previously used for FMRP eCLIP-seq, and confirmed expected loss of FXR1P expression (FIG. 2C). FXR1^{-/-} hPSCs were then differentiated into neurons and FMRP eCLIP-seq was performed to probe FMRP targets in the absence of FXR1P, again using FMR1^{y/-} peak subtraction (FIG. 2D, Table 3). This allowed comparison of FMRP targets in FXR1^{-/-} neurons (FIGS. 2A-2F) with FMRP targets in FXR1^{-/-} neurons (FIG. 2D). Notably, FXR1P was required for three-quarters of FMRP binding events, as 74.4% of FMRP targets were lost in FXR1^{-/-} neurons and 25.6% of FMRP targets were retained in FXR1^{-/-} neurons (FIG. 2E). A small number of targets were gained by FMRP upon FXR1P loss (FIG. 2E), which could be due to changes in the underling transcriptional landscape. Importantly, FMRP intronic peaks were substantially reduced upon FXR1P loss (FIG. 2F). These data indicate that FXR1P is a critical co-factor for a majority of FMRP binding events in neurons, including a majority of pre-mRNA binding events.

TABLE 3
Summary of final FMRP eCLIP-seq dataset in FXR1−/− neurons following FMR1y/−
peak subtraction

| | | Significant | | | |
|---|---|---|---|---|---|
| Cell Type | Antibody | Peaks | Genes | Cell Input | % Subtracted |
| Neurons | Ab17722 | 3120 | 1118 | 7.0E+07 | 33.5 |

### Example 3: FMRP Targets are Enriched for Key Neurodevelopmental Disease Associated Genes

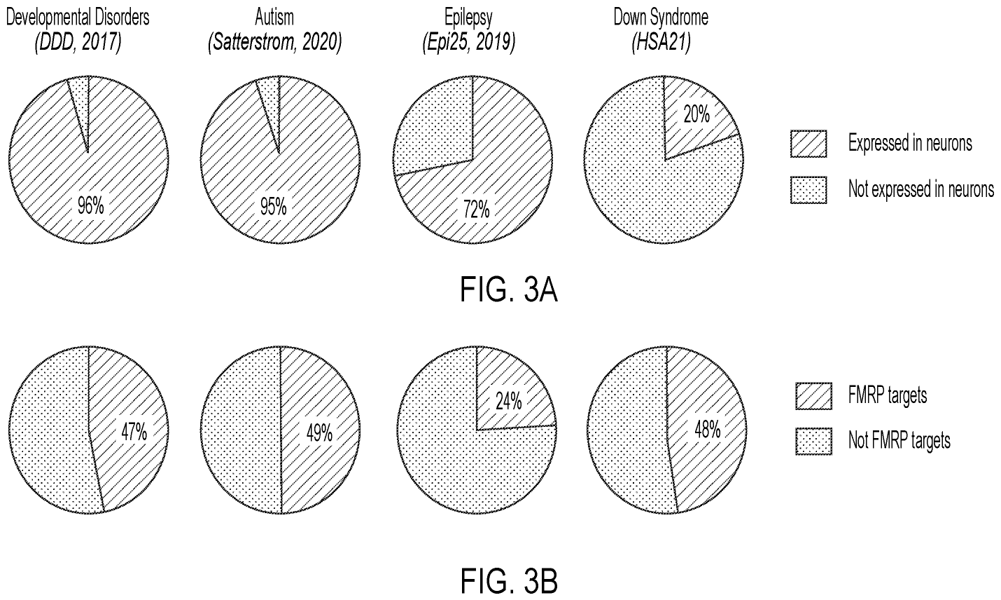

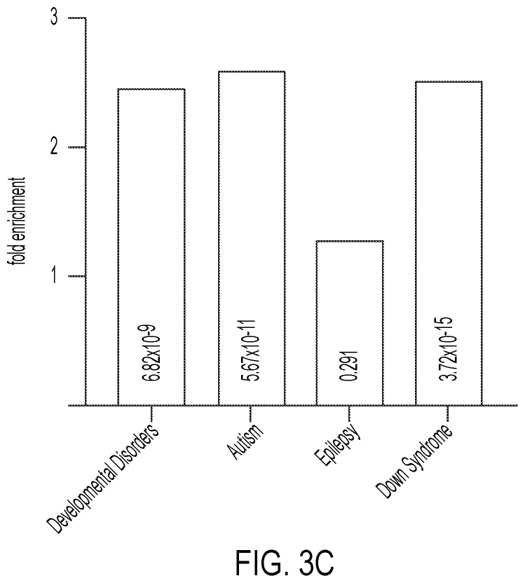

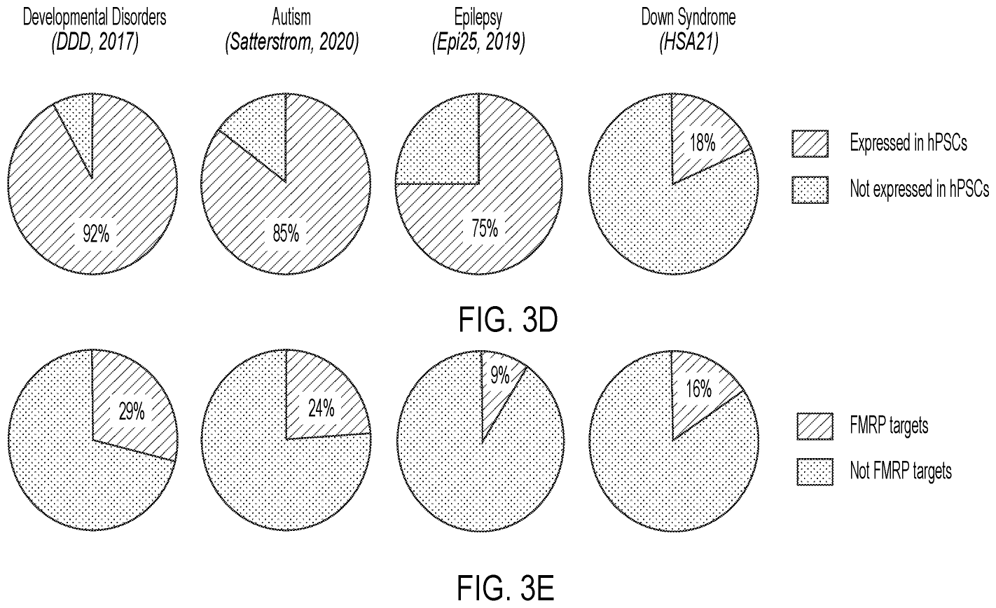

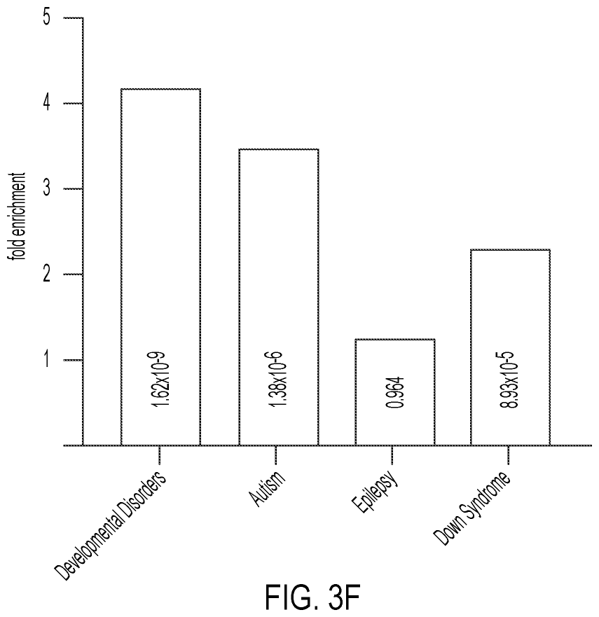

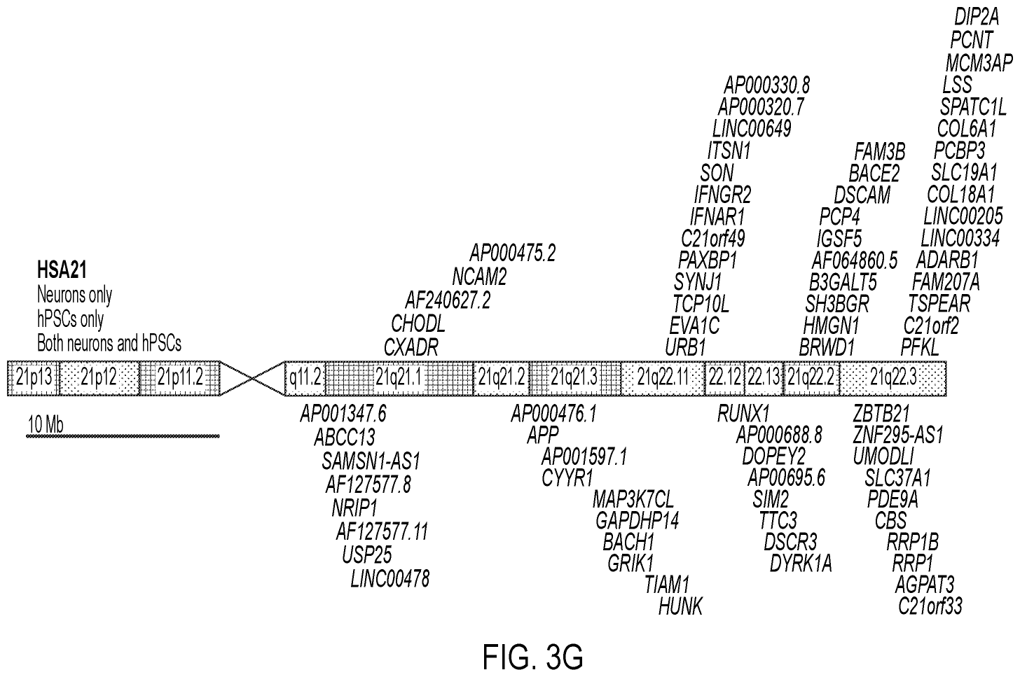

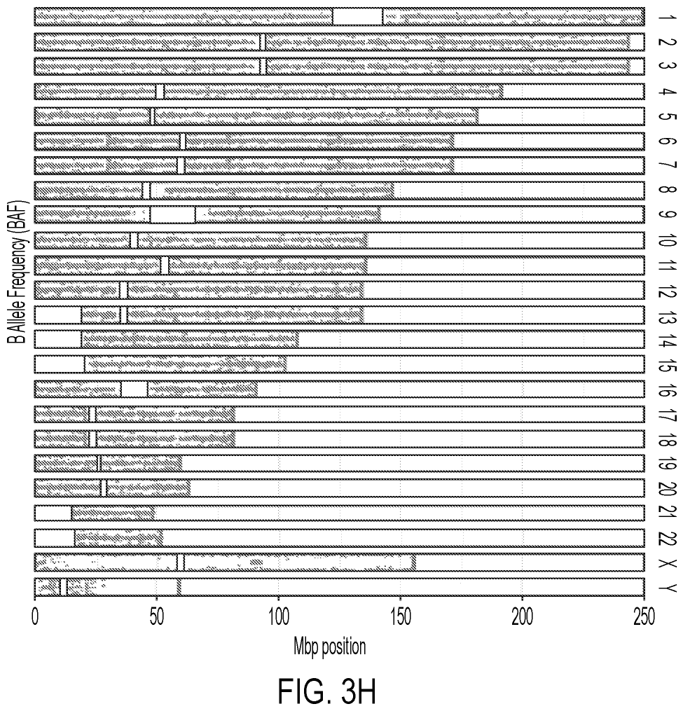

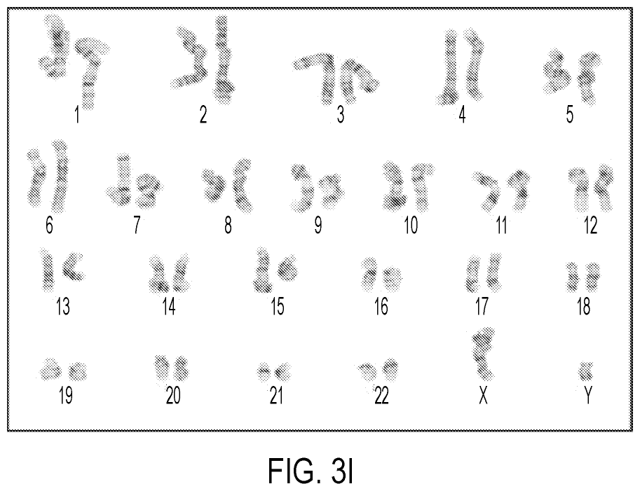

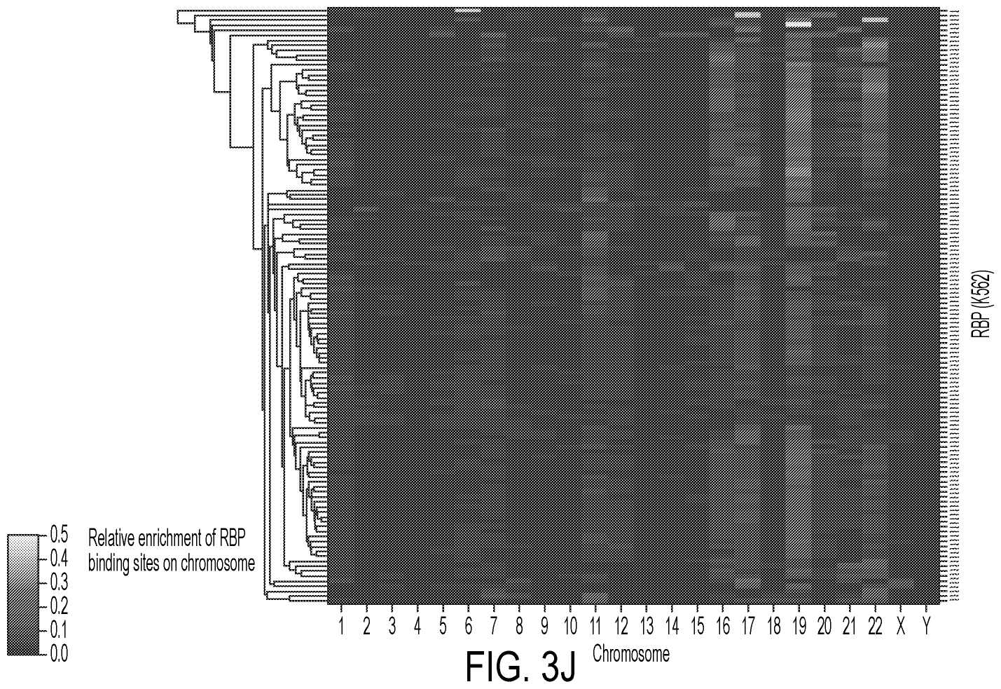

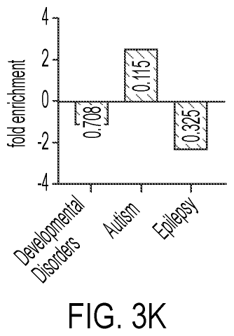

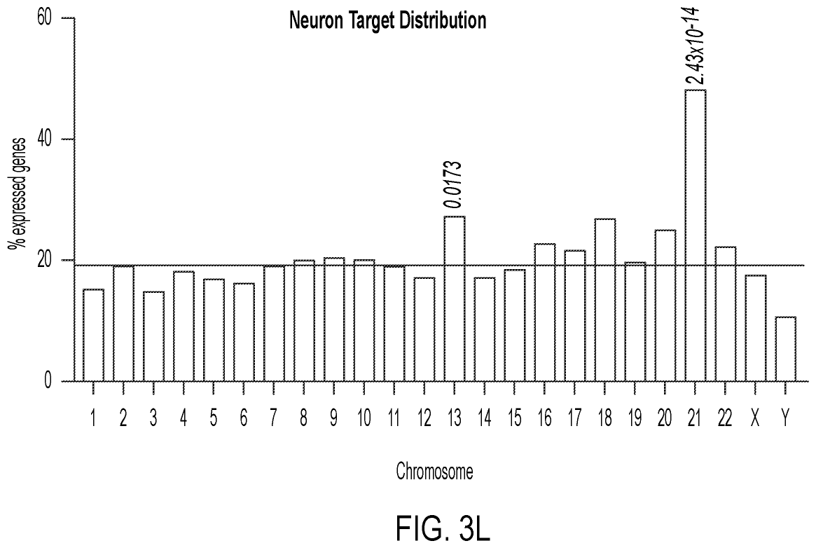

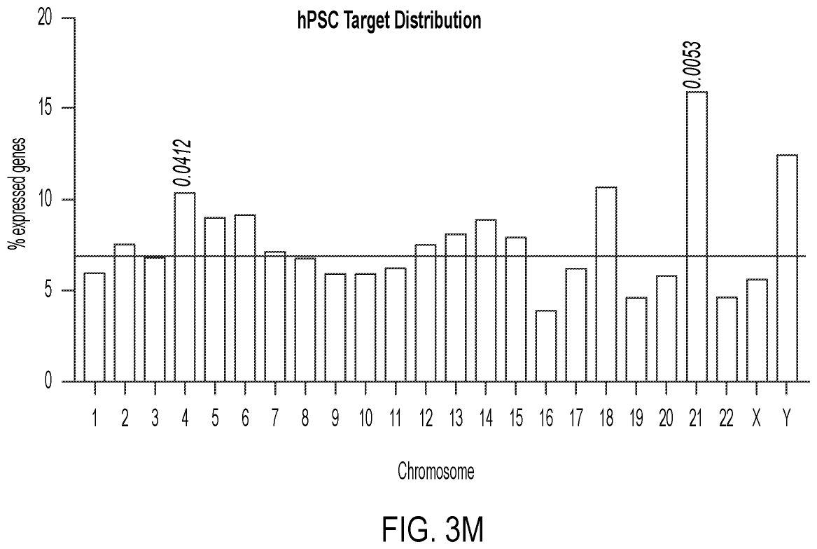

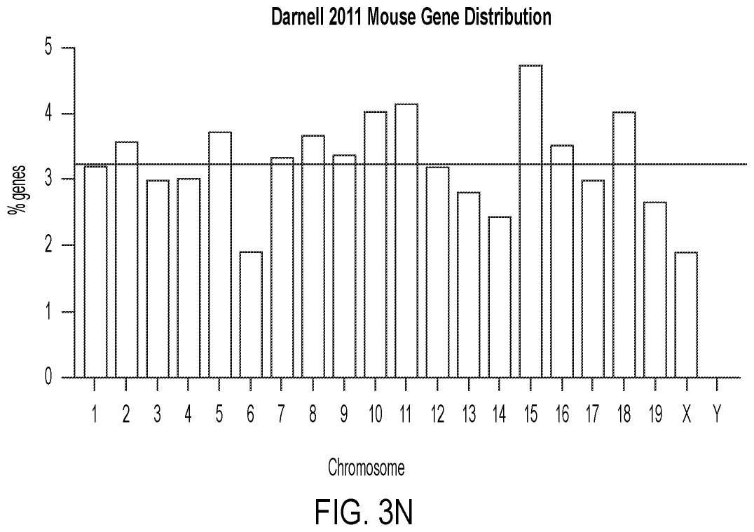

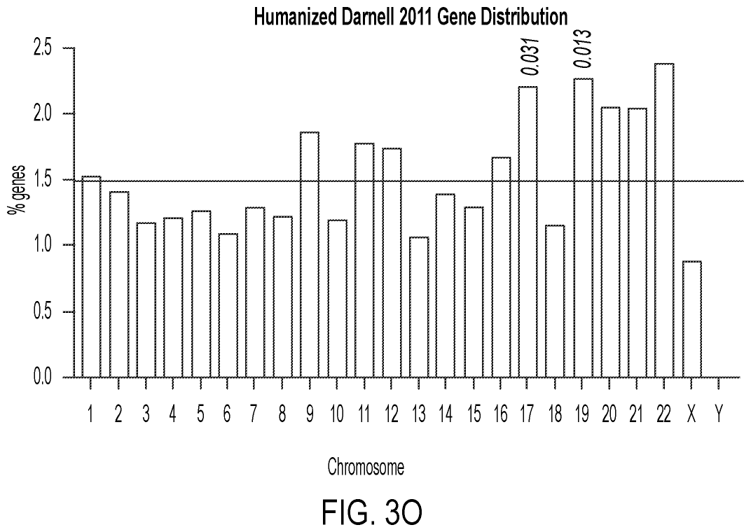

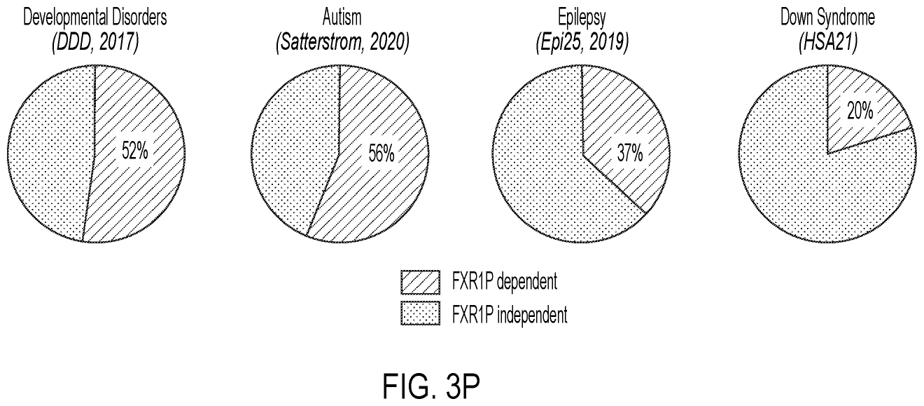

It was next assessed whether FMRP targets in human neurons would show significant enrichment for neurodevelopmental disease associated genes (FIGS. 3A-3C), as reported for FMRP targets from mouse brain (Iossifov, I. et al., Nature, 515, 216-21 (2014), Sanders, S. J. et al., Neuron 87, 1215-33 (2015), Schizophrenia Working Group of the Psychiatric Genomics, Nature 511, 421-27 (2014)). Notably, 96% of genes implicated in developmental disorders and 95% of high-confidence autism susceptibility genes identified through exome sequencing were expressed in human neurons (FIG. 3A), consistent with studies indicating that excitatory neurons are enriched for the expression of autism risk genes. Moreover, nearly half of all expressed genes from each disease dataset were FMRP targets (FIG. 3B); FMRP targets in human neurons were 2.47-fold enriched (p=5.11×10⁻⁹) for genes implicated in developmental disorders and 2.59-fold enriched (p=4.25×10⁻¹¹) for autism susceptibility genes (FIG. 3C). These data are consistent with previous studies showing that FMRP targets are enriched for genes independently implicated in neurodevelopmental diseases, and expand upon the specific overlapping targets with relevance to the developing human brain. These data also support the use of human in vitro derived excitatory neurons for study of neurodevelopmental disease mechanisms.

Unexpectedly, a substantial number of FMRP targets transcribed from HSA21 and implicated in DS disease biology were also noted, such as APP, DYRK1A, NCAM2, PCP4, DSCAM, BACE2, BACH1 and RUNX1. While 20% of genes encoded on HSA21 were expressed in human neurons (FIG. 3A), nearly half of those expressed genes were FMRP targets (FIG. 3B). Specifically, FMRP targets in human neurons were 2.51-fold enriched (p=2.79×10⁻′) for HSA21-encoded transcripts, closely paralleling results obtained for developmental disorders and autism (FIG. 3C). As expected, fewer disease implicated genes were expressed in hPSCs although FMRP targets were still enriched for genes implicated in developmental disorders, autism and Down syndrome in this cell type (FIGS. 3D-3F).

These results indicate that FXS and DS may share some underlying molecular mechanisms during early brain development as previously proposed for FXS and other neurodevelopmental diseases. FMRP RNA targets were distributed across the q-arm of HSA21 and did not cluster in a specific cytoband (FIG. 3G). Cells used for eCLIP-seq did not have chromosomal aberrations by G-band analysis or high-density SNP array (FIGS. 3H, 3I). Additionally, existing eCLIP-seq datasets for 120 RNA binding proteins in K562 leukemic cells available through the ENCODE consortium (E. P. Consortium, Nature 489, 57-74 (2012)) were leveraged to confirm a lack of widespread HSA21 binding events in eCLIP-seq datasets generally (FIG. 3J), See, also, FIGS. 3K-3P).

Collectively, these data indicate that FMRP binds a significant number of neurodevelopmental disease-associated genes in human neurons. Moreover, the discovery described herein that FMRP targets are enriched for genes transcribed from HSA21, provides a potential molecular link between FXS and DS.

### Example 4: FXS and DS Converge on Shared Transcriptional Perturbations

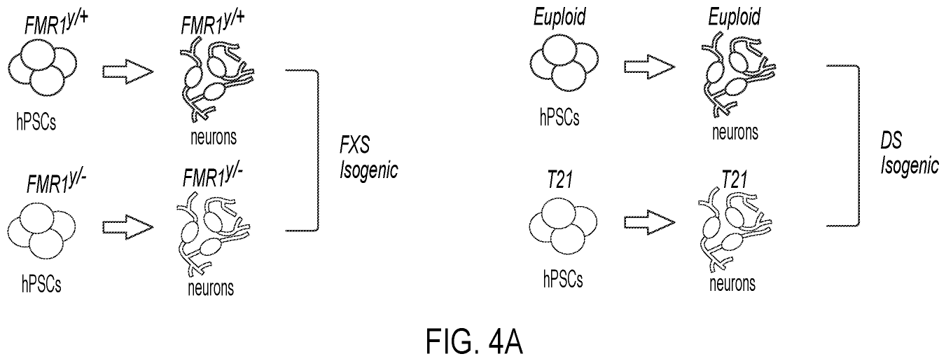

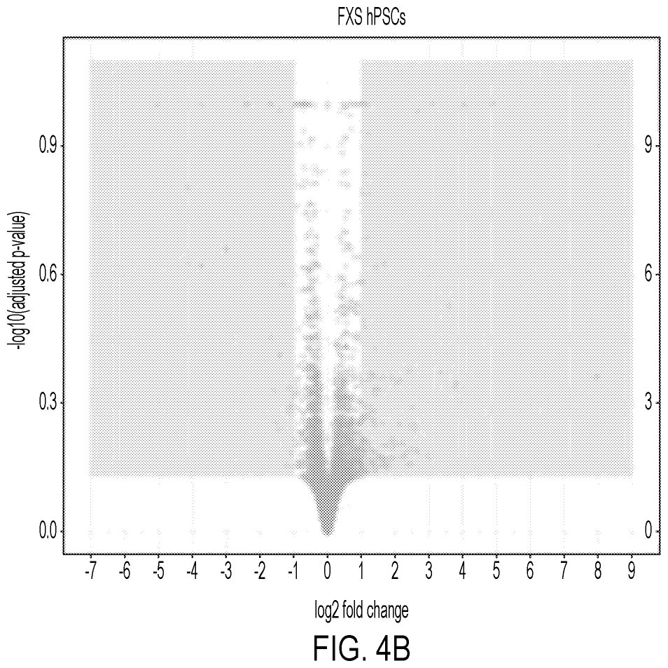

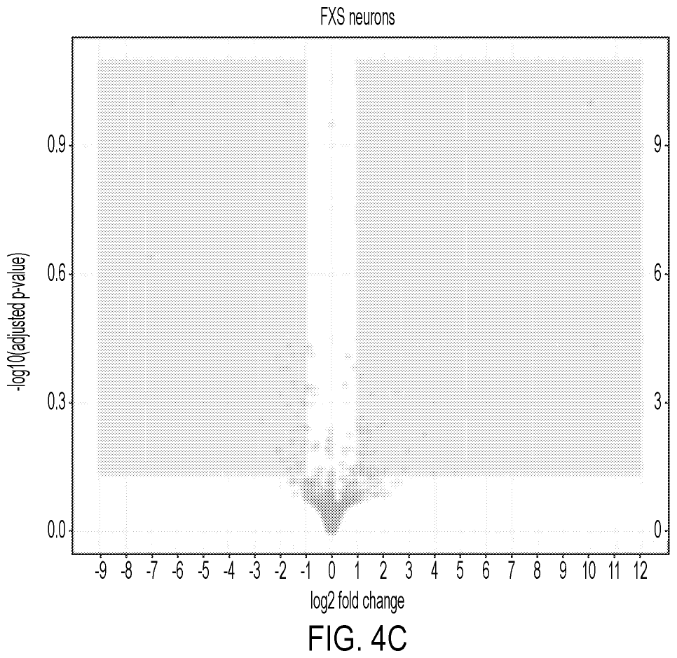

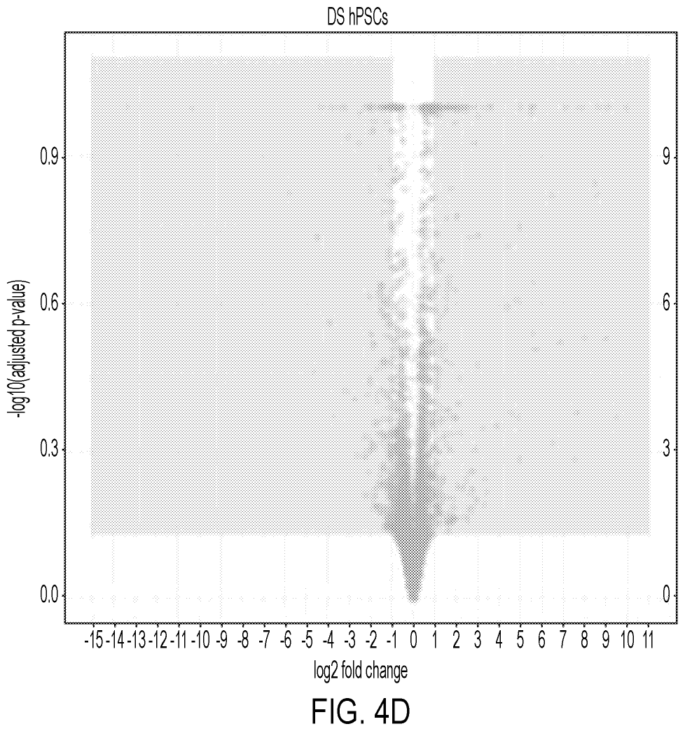

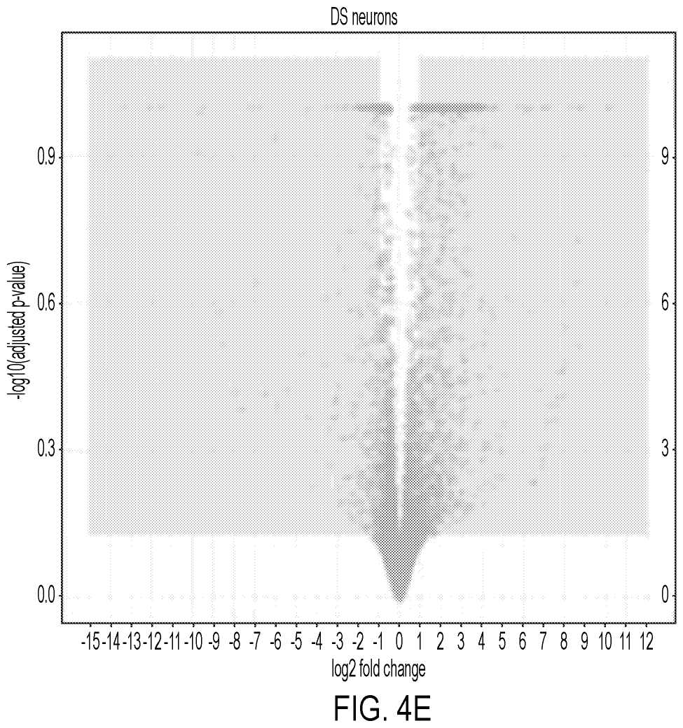

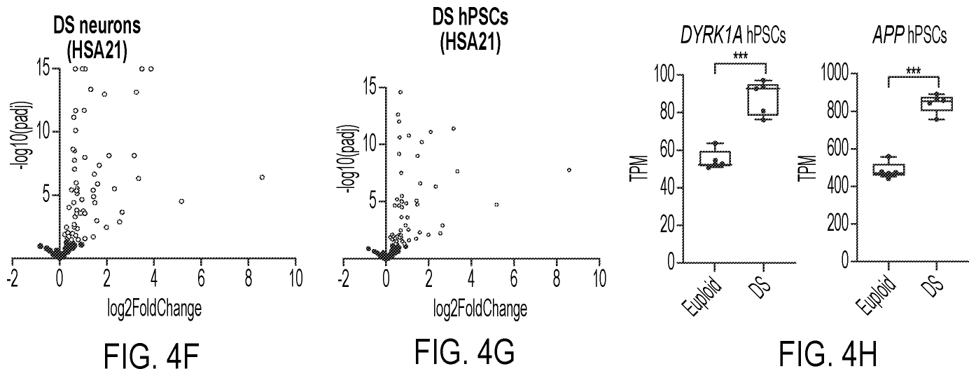

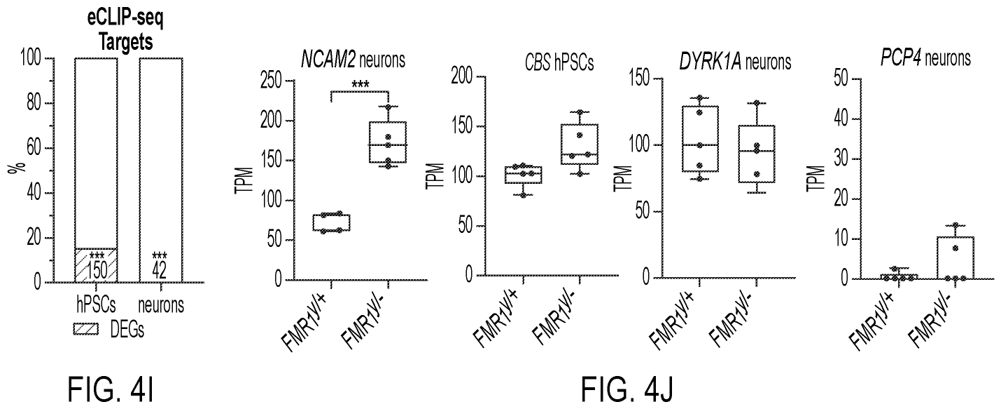

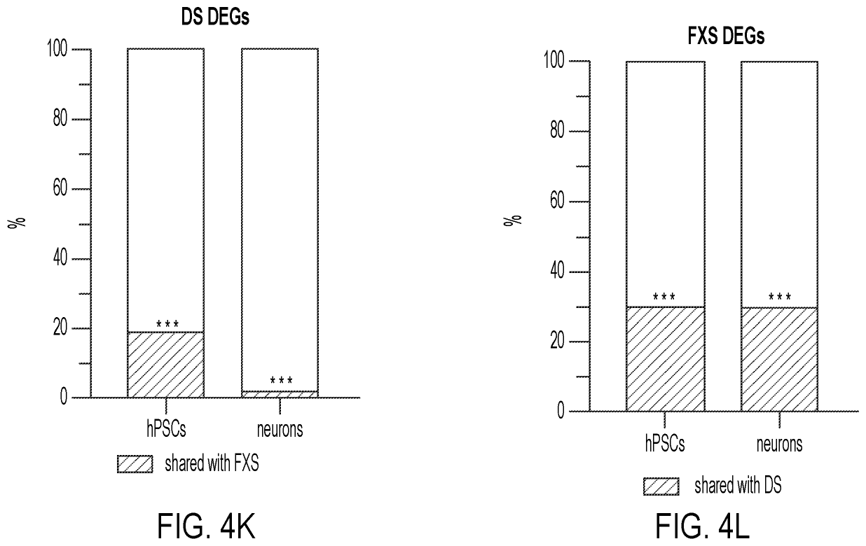

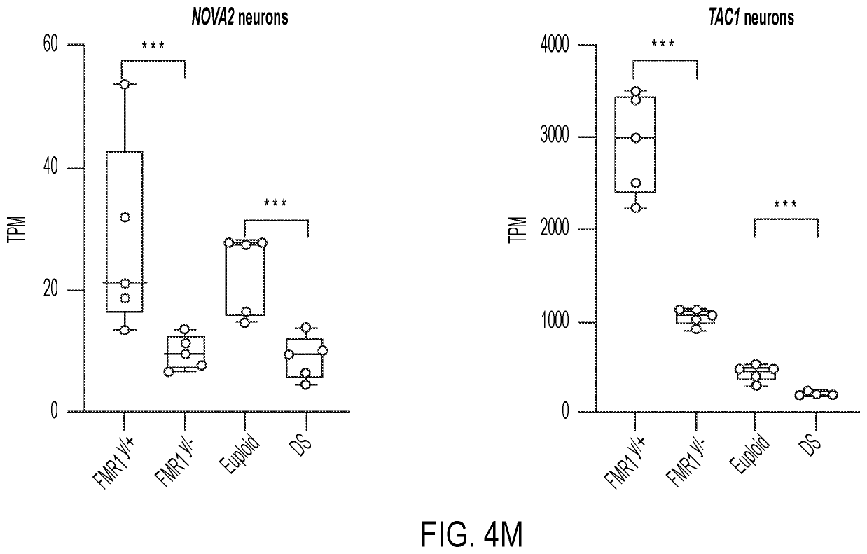

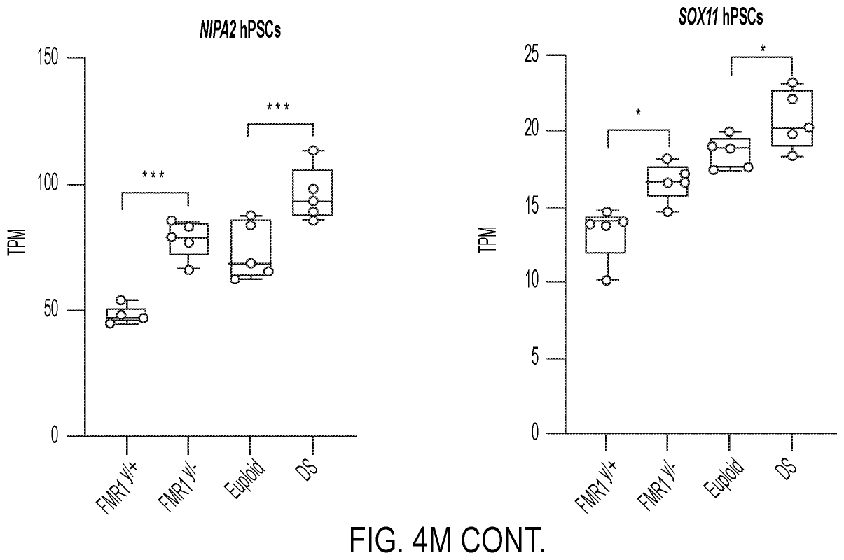

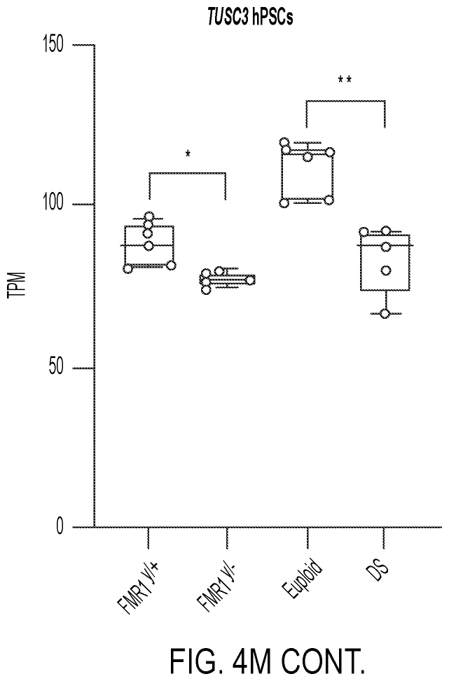

The above FMRP target analyses support a novel model whereby FXS and DS can result in the perturbation of a significant number of the same genes through different mechanisms. However, the data described herein combined with previous studies also support complex roles for FMRP in different stages of RNA processing (Verheij, C. et al., Nature, 363, 722-724 (1993); Zhou, L. T. et al., Neuroscience, 349, 64-75 (2017); Tran, S. S. et al., Nature neuroscience, 22, 25-36 (2019); Alpatov, R. et al., Cell 157, 869-881 (2014); Chakraborty, A. et al., bioRxiv (2019); Kim, M. et al., Mol Cell Biol, 29, 214-228 (2009); D'Souza, M. N. et al., iScience, 9, 399-411 (2018); Edens, B. M. et al., Cell reports, 28, 845-854 e845 (2019); Dury, A. Y. et al., PLOS Genet, 9, e1003890 (2013); Taha, M. S. et al., LOS one, 9, e91465 (2014)), making it challenging to predict how FMRP loss would impact the expression or function of any individual target. Therefore, global transcriptional perturbations were first quantified in FXS and DS cellular models to broadly assess molecular convergence. Specifically, RNA-seq was performed using an isogenic pair of DS patient and euploid control iPSC lines (generated from a mosaic patient fibroblast line) (Weick, J. P. et al., PNAS, 110, 9962-9967 (2013)), as well as the isogenic FMR1^{y/+} and FMR1^{y/-} lines (Susco, S. G. et al., Developmental Biology, 468:93-100 (2020)) examined transcriptional changes in both pluripotent stem cells and neurons (FIGS. 4A-4F).

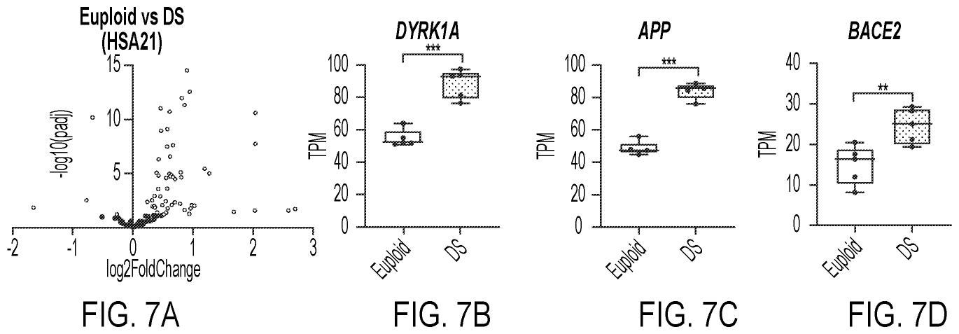

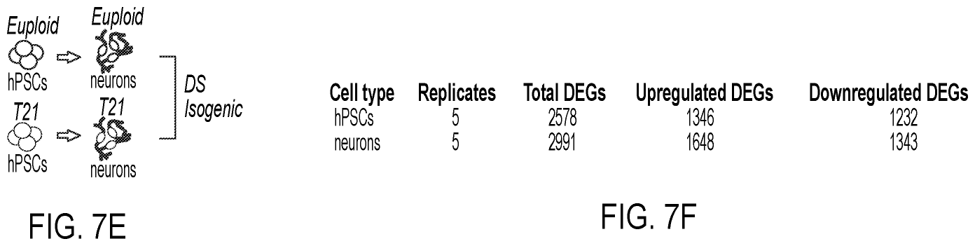

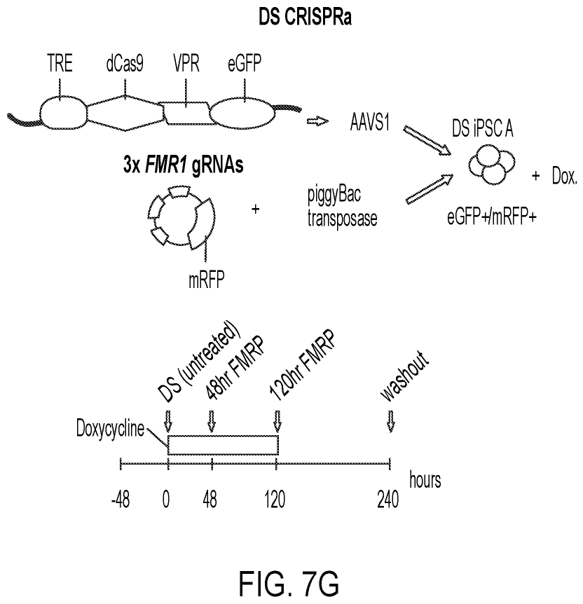

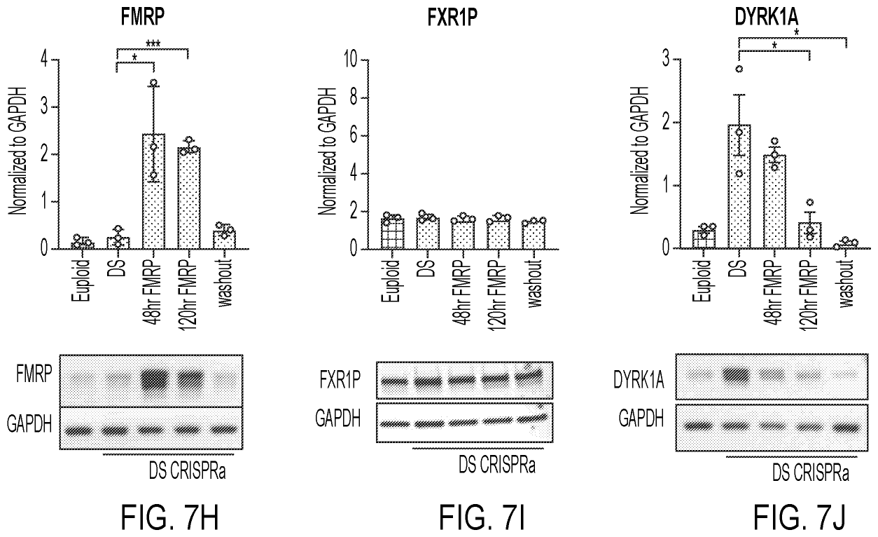

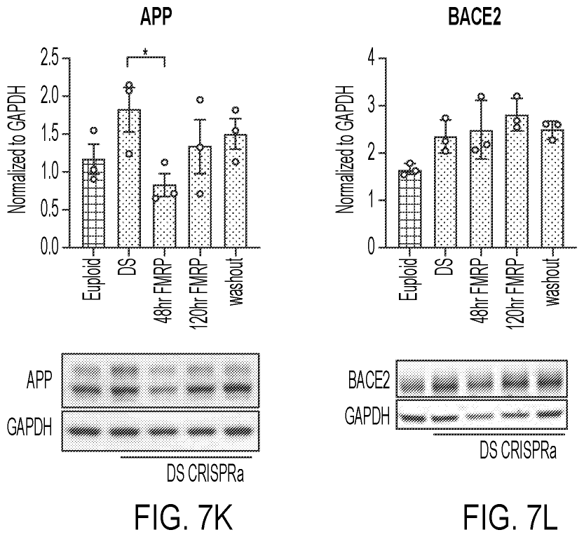

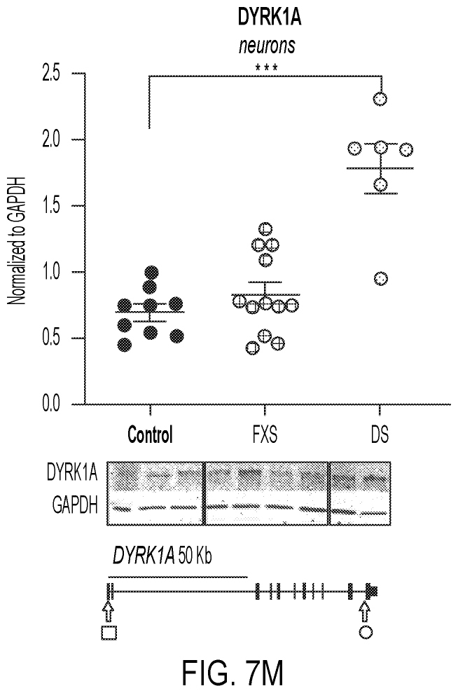

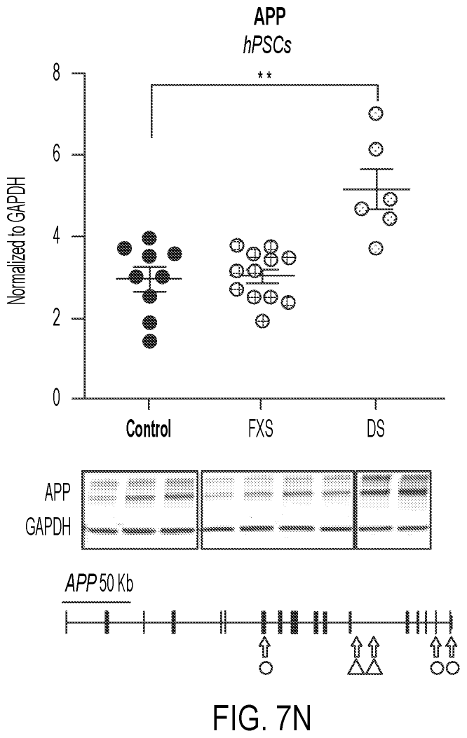

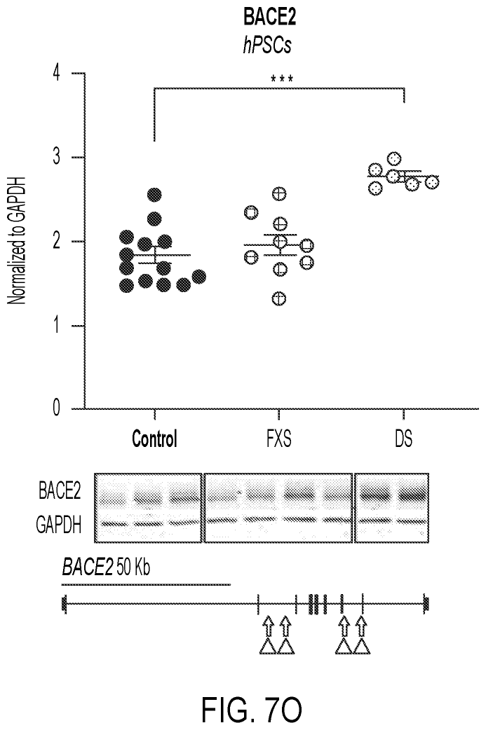

Within each cell type and disease state comparison, roughly equal numbers of genes were observed being significantly upregulated and downregulated (FIGS. 4B-4F, Table 4). As expected, an extra copy of HSA21 led to significant upregulation of HSA21-encoded transcripts in both hPSCs (FIGS. 4G and 7A, 4H) and neurons (FIGS. 4B-4F). Cross-referencing the FMRP eCLIP-seq data with genes differentially expressed following FMR1 loss revealed that a small but significant fraction of FMRP targets were differentially expressed at the transcript level following FMR1 loss (FIG. 41). For example, the FMRP target NCAM2 was significantly upregulated at the transcript level following FMR1 loss (p=0.000154), while the FMRP targets CBS, DYRK1A and PCP4 remained unchanged (FIG. 4J).

TABLE 4
Summary of RNA-seq datasets

| | Isogenic | Biological | | Upregulated | Dowregulated |
|---|---|---|---|---|---|
| Cell Type | Pair | Replicates | Total DEGs | DEGs | DEGs |
| hPSCs | FXS | 5 | 1614 | 819 | 795 |
| Neurons | FXS | 5 | 130 | 62 | 68 |
| hPSCs | DS | 5 | 2578 | 1346 | 1232 |
| Neurons | DS | 5 | 2991 | 1648 | 1343 |

These data indicate that FMRP modulates RNA expression or stability of a minority of its targets, and thus a majority of genes differentially expressed following FMRP loss may result from indirect, secondary changes as opposed to direct FMRP binding. Importantly, significant overlap was found in differential gene expression patterns between FXS and DS hPSC (p=3.19×10⁻³³) and neuron (p=0.00495) models (FIGS. 4K, 4L). In DS models, although thousands of genes were significantly differentially expressed, 18.5% of transcriptional changes in hPSCs were shared with FXS (FIG. 4K). In FXS models, 29.5% of significant DEGs in hPSCs and 29.2% of significant DEGs in neurons were also significantly perturbed in DS (FIG. 4L). For example, NOVA2 and TAC1 transcript expression levels were significantly downregulated in neurons derived from both FXS and DS models compared to controls (FIG. 4M). Similarly, in hPSCs, NIP2A and SOX11 transcript expression levels were significantly upregulated while TUSC3 transcript expression levels were significantly downregulated in both FXS and DS models compared to controls (FIG. 4M).

NOVA2, NIPA2, SOX11 and TUSC3 are notable for their independent associations with neurodevelopmental diseases. All four genes were coordinately misregulated at the transcript level in FXS and DS models; NOVA2 was an FMRP target from HSA19 while NIPA2, SOX11 and TUSC3 were neither FMRP targets nor localized to HSA21, suggesting that FXS and DS can converge on shared transcriptional perturbations through direct or indirect mechanisms.

### Example 5: FSX and DS Converge on Shared Proteomic Perturbations

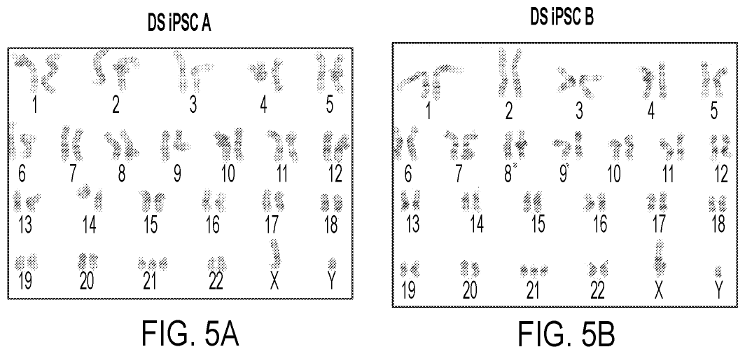

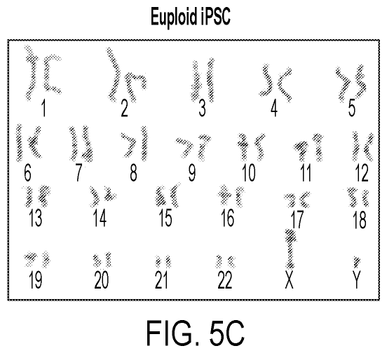

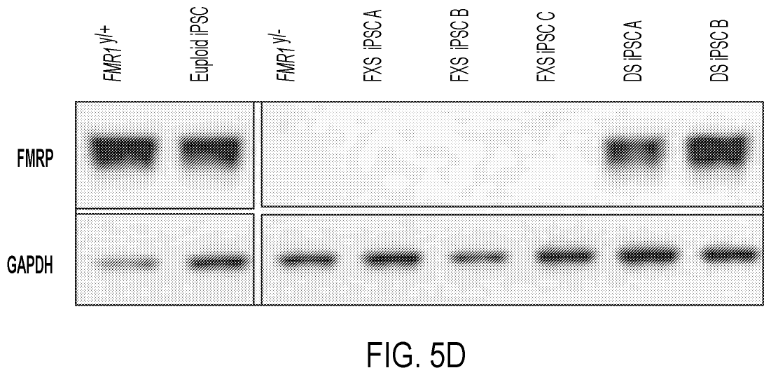

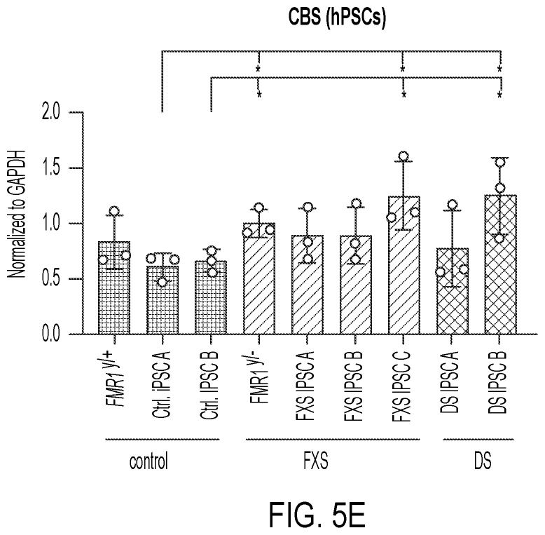

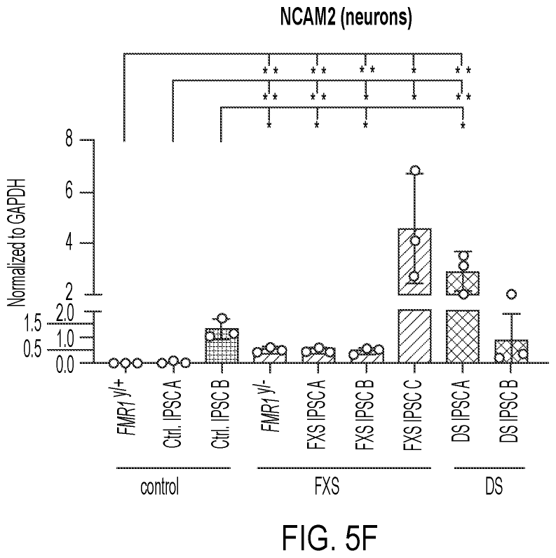

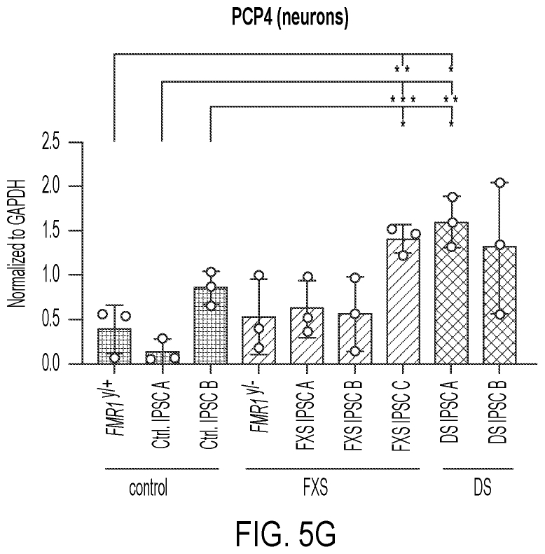

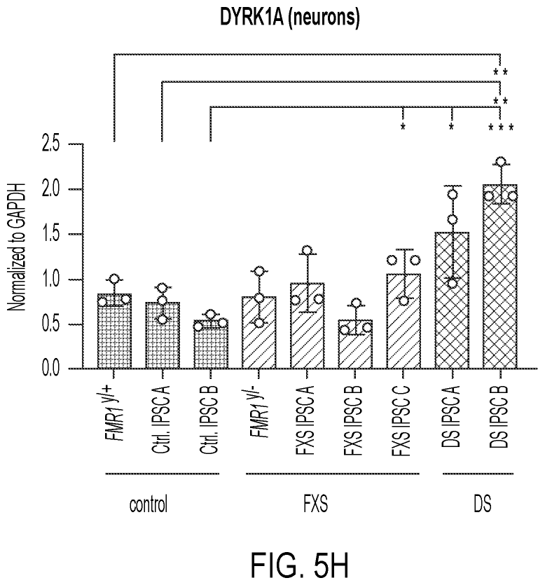

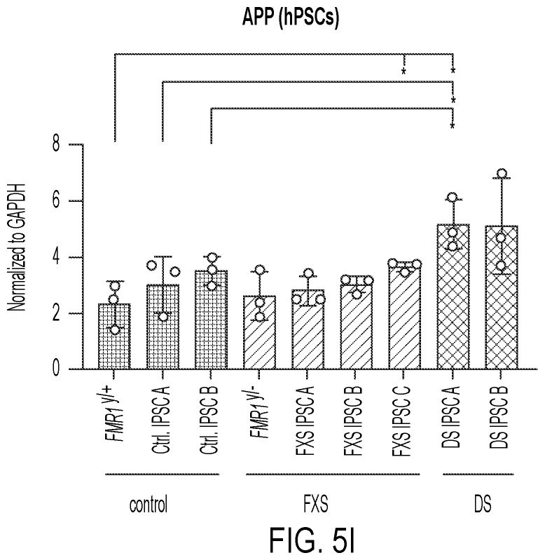

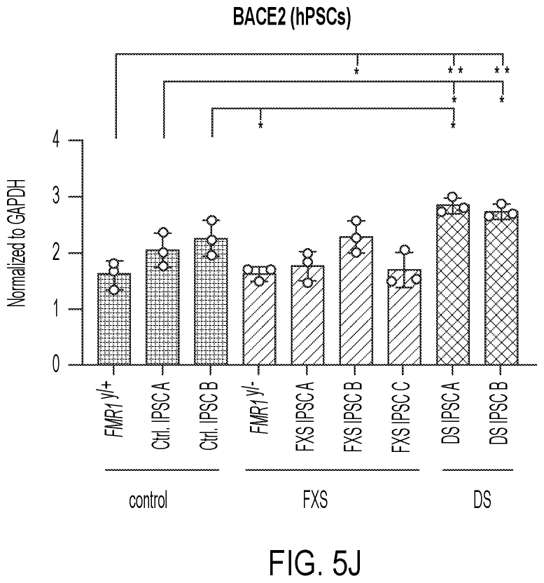

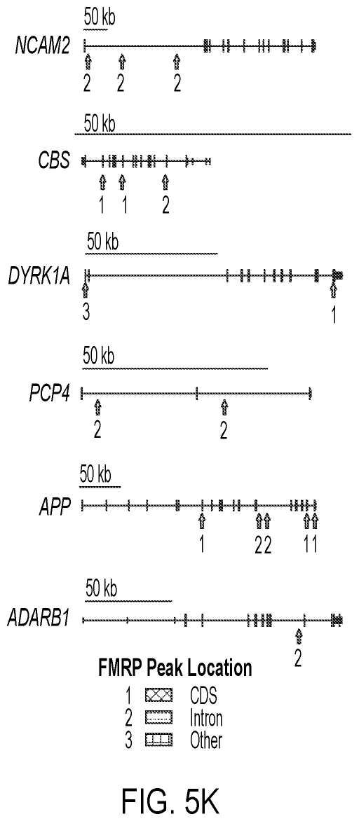

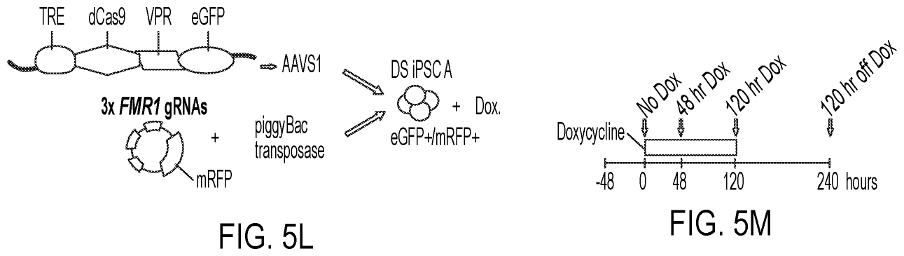

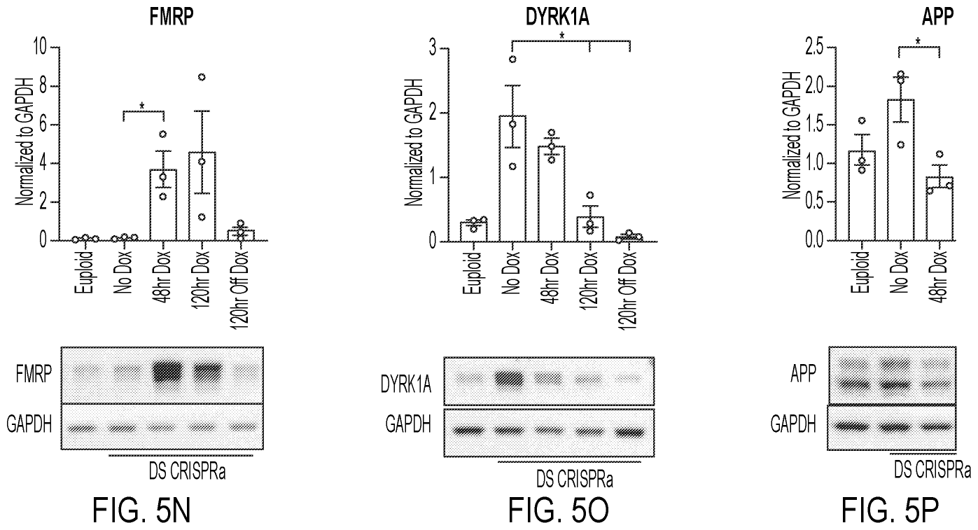

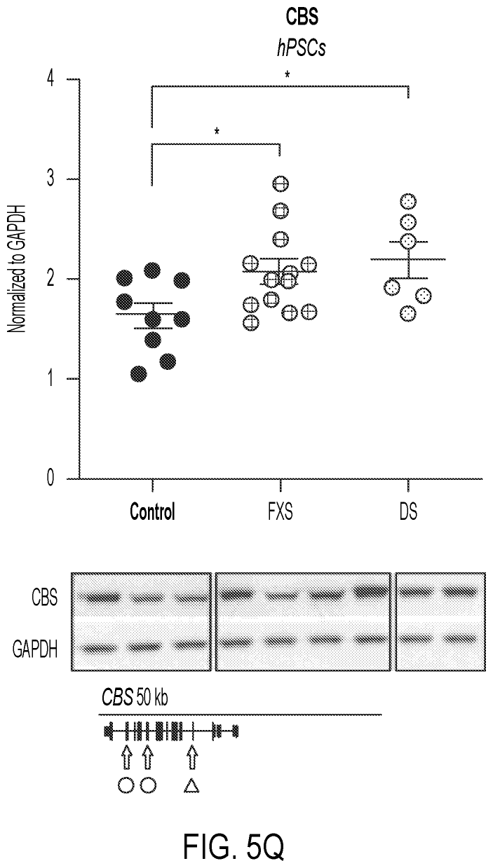

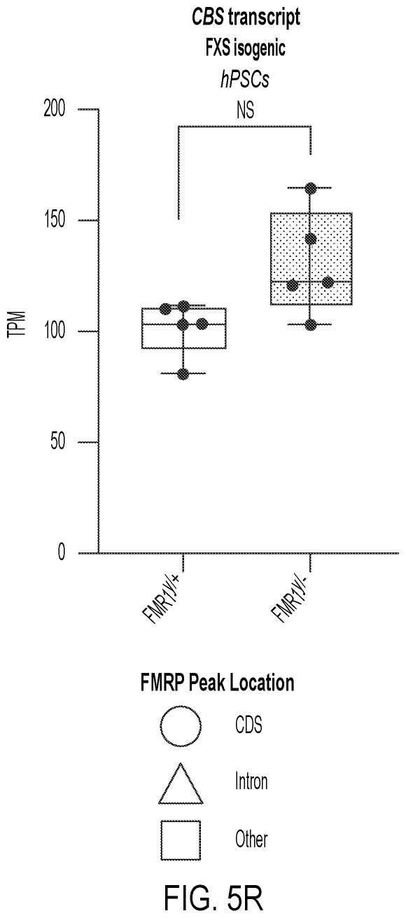

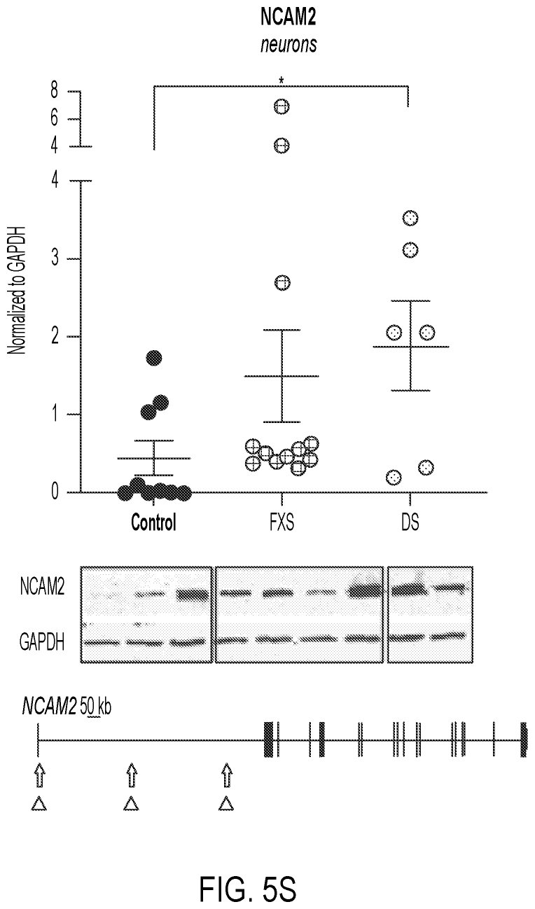

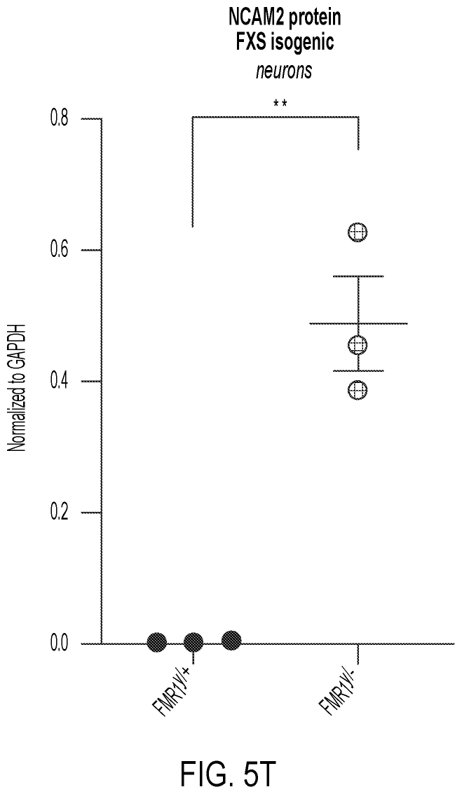

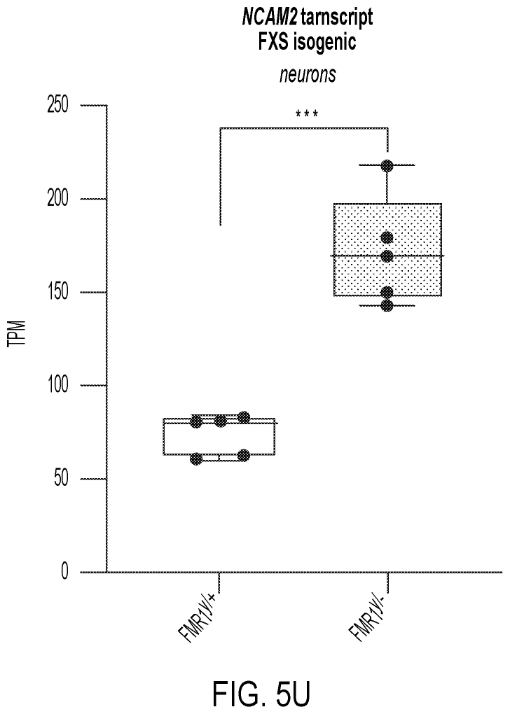

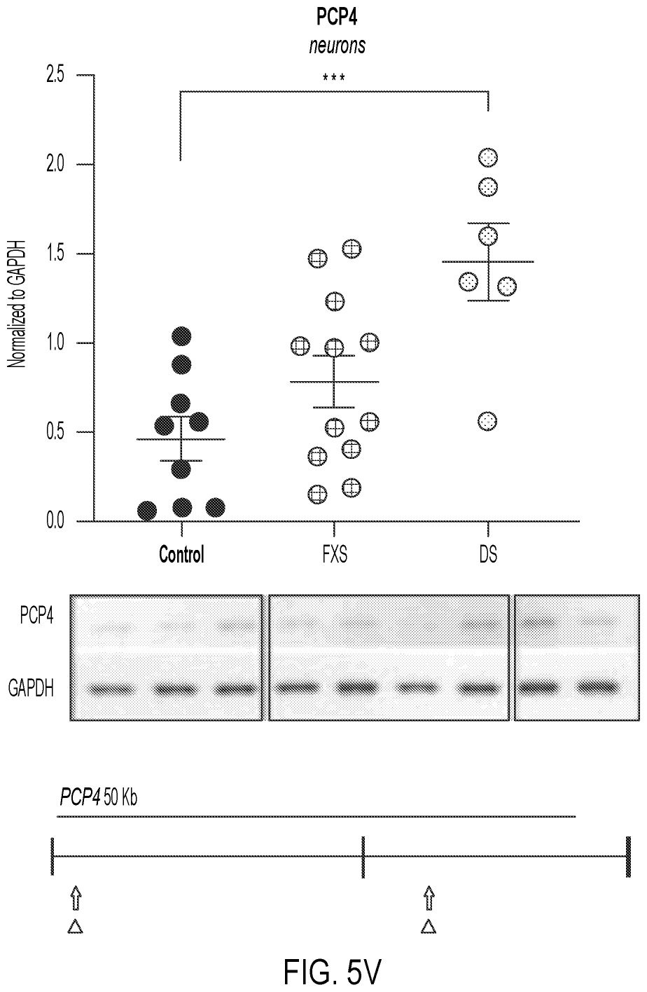

While studies using human fetal and adult post-mortem brain tissue consistently report an overall increase in expression of genes transcribed from HSA21 in DS patients compared to euploid controls, a limited number of genes have been assessed at the protein level. Therefore, DS and FXS patient and control iPSC lines were generated or acquired and protein expression levels of a set of individual HSA21-encoded FMRP targets was assessed across multiple genetic backgrounds, focusing on targets implicated in specific aspects of DS disease biology. Specifically, a set of three iPSCs reprogrammed from FXS patient fibroblast lines were used and two DS patient iPSC lines were acquired plus a euploid iPSC control in addition to the isogenic FMR1^{y/+} and FMR1^{y/-} CRISPR engineered hPSC lines (FIGS. 5A-5D). Protein expression levels were then assayed for six FMRP targets encoded on HSA21: NCAM2 (cell adhesion molecule), CBS (enzyme in the transsulfuration pathway), DYRK1A (tyrosine kinase), PCP4 (calmodulin binding protein), APP (amyloid precursor protein) and ADARB1 (RNA editing enzyme) (FIGS. 5E-5J).

Notably, CBS protein expression levels were significantly upregulated in both FXS (p=0.0251) and DS (p=0.0213) patient cells compared to controls (FIG. 5Q). As demonstrated by the eCLIP-seq and RNA-seq data, FMRP bound CBS in both CDS and intron regions (FIG. 5Q). No evidence was found for altered CBS transcript levels following FMRP loss (FIG. 5R), suggesting that CBS may be regulated at the translational level but not at the level of transcript abundance or stability. While NCAM2 protein expression levels were significantly upregulated across DS (p=0.0180) but not FXS (p=0.1597) patient cell lines using grouped analyses (FIG. 5S), large inter-individual differences were noted, with some individual control—FXS cell line comparisons showing significant upregulation of NCAM2 (FIG. 5F). Therefore, the isogenic FMR1^{y/+} and FMR1^{y/-} comparison was extracted, revealing significant NCAM2 protein upregulation following FMRP loss (p=0.0024), (FIG. 5T). Here, the signal may have been obscured by the broad distribution of NCAM2 protein expression levels observed across individuals. Results for individual cell line comparisons for all proteins queried are shown in FIGS. 5E-5J. Interestingly, only FMRP binding events were detected in an intron of NCAM2 (FIG. 5S), and NCAM2 was significantly increased at the transcript level following FMRP loss (p=0.000154), (FIG. 5U), suggesting that FMRP negatively regulates NCAM2 stability or expression which then impacts protein levels. Importantly, potential biological roles for CBS and NCAM2 in FXS have not been investigated, and the results described herein confirm protein level changes for FMRP targets with reported roles in DS disease biology. Overexpression of NCAM2 reportedly leads to decreased synaptic maturation and dendritic spine formation in DS mouse models. In view of the findings herein, it can now be assessed whether the elevated NCAM2 expression observed in FXS models as described herein could similarly contribute to FXS-associated dendritic spine defects. NCAM2 has also been reported to be implicated in developmental delay, as well as in synaptic dysfunction in Alzheimer's disease, which may indicate broader roles in developmental or degenerative disease. Overexpression of CBS is associated with metabolic dysfunction in DS, and is reportedly necessary and sufficient for induction of a subset of cognitive phenotypes in mouse models. Accordingly, elevated CBS may similarly contribute to metabolic or cognitive dysfunction observed in FXS.

Other targets were significantly upregulated at the protein level in DS patient cell lines compared to controls including PCP4 (p=0.0008), DYRK1A (p<0.0001), APP (p=0.0012) and BACE2 (p=0.0002), (FIG. 5J, 5V and FIGS. 7S, 7T and 7U), but did not show evidence for significant mis-regulation across FXS cell lines. Examples of significant protein upregulation in individual FXS patient cell lines were not observed compared to non-isogenic controls, most notably for PCP4 (FIG. 5G), but this did not reach significance using grouped analyses or isogenic pairs. While upregulated APP protein expression in FXS mouse models has been reported, the experiments and data described herein do not support broad upregulation of APP across FXS hPSC models (FIG. 7T and FIG. 51). As expected, protein-level effect sizes in both diseases were modest. Collectively, the data described herein demonstrate that DS and FXS can indeed lead to shared perturbations of the same target genes through different mechanisms, and identify CBS and NCAM2 as novel, high-priority targets for additional functional interrogation in FXS based on their known biological roles in DS.

### Example 6: Acute Upregulation of Endogenous FMRP in DS Patient Cells Normalizes Protein Expression Levels of Key HSA21-Encoded Targets

Although some HSA21-encoded FMRP targets like DYRK1A were significantly upregulated in DS but not FXS (FIG. 5G), it was reasoned that increasing FMRP dosage in the context of DS could potentially further modulate target expression. Therefore, an inducible CRISPR activation (CRISPRa) construct was stably introduced into the AAVS1 safe-harbor locus of a DS patient iPSC line, along with a multiplexed piggyBac integration plasmid encoding three FMR1 activating gRNAs (DS-CRISPRa) (FIG. 5L). As expected, doxycycline induction of the DS-CRISPRa cell line led to efficient and transient upregulation of FMRP protein expression (FIG. 5M). Strikingly, acute upregulation of endogenous FMRP was sufficient to significantly decrease protein expression levels of DYRK1A in the DS-CRISPRa line (FIG. 5O). Here, transient FMRP upregulation led to a sustained reduction in protein expression that lasted beyond withdrawal of doxycycline and normalization of FMRP levels (FIG. 5N).

It was determined that a significant reduction in APP protein expression was achieved, but in this case, APP levels were more transiently reduced and recovered by the 120-hour time-point (FIG. 5P; FIG. 51). FMRP bound DYRK1A in both CDS and non-coding exon regions and APP in both CDS and intron regions (FIG. 5C), again leaving open multiple possible mechanisms of gene regulation. Importantly, these data provide proof-of-concept that transiently increasing endogenous FMRP in the context of DS is sufficient to significantly modulate key HSA21-encoded targets.

Given that HSA21-encoded transcripts are increased in abundance in DS due to the extra copy of the chromosome, and FMRP regulates a large number of these transcripts, it was hypothesized that increasing FMRP dosage in the context of DS would modulate expression of HSA21-encoded targets. Using CRISPRa in two independent DS patient iPSC lines, endogenous FMRP was acutely upregulated and normalized protein expression levels of multiple FMRP targets, including APP (amyloid-B precursor protein) and DYRK1A. Increased APP expression is associated with the highly elevated rates of Alzheimer's disease in DS patients (DS affects 1 in 700 and by age 40, almost all patients have amyloid plaques). DYRK1A is independently implicated in autism and intellectual disability.

It is critical to emphasize that existing knowledge of molecular mechanisms underlying FXS and DS have failed to translate into effective therapeutic strategies. Indeed, there are currently no effective treatments for FXS or DS, which may be due in part to the large number of genes thought to be simultaneously and subtly disrupted in each disease. By defining high-confidence FMRP targets for the first time in two physiologically relevant human cell types, novel molecular overlap between FXS and DS was uncovered, paralleling results for FXS and other neurodevelopmental diseases. Without wishing to be bound by theory, while many gene targets and patient phenotypes do not overlap in FXS and DS, it is proposed that genes misregulated in both diseases are candidates for contributing to the shared aspects of disease pathology, which includes intellectual disability, increased rates of autism, and increased rates of other mental health disorders compared with the general population. The finding of shared molecular perturbations, combined with data showing that acutely upregulating endogenous FMRP can modulate the key HSA21-encoded targets DYRK1A and APP, suggests there may be opportunities for shared therapeutic intervention in FXS and DS.

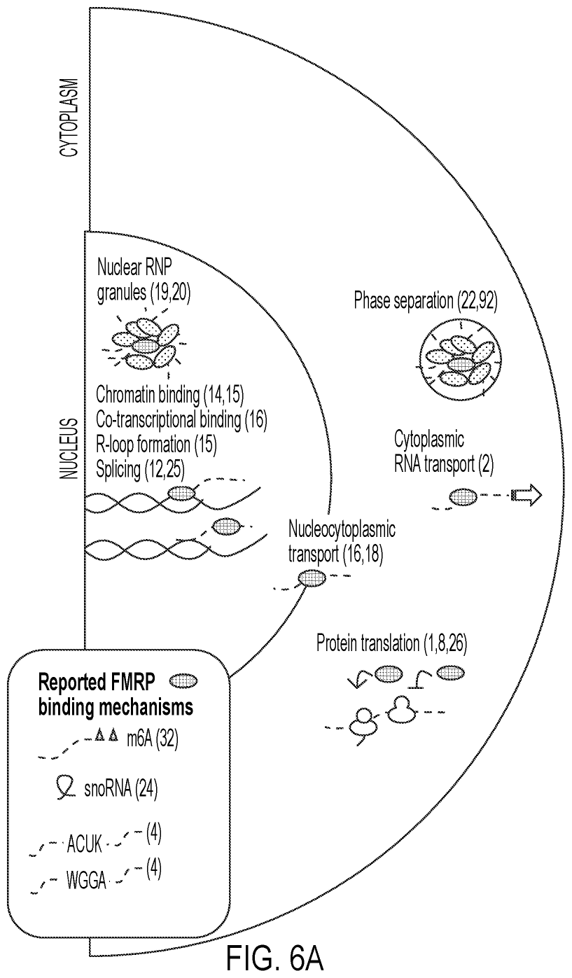

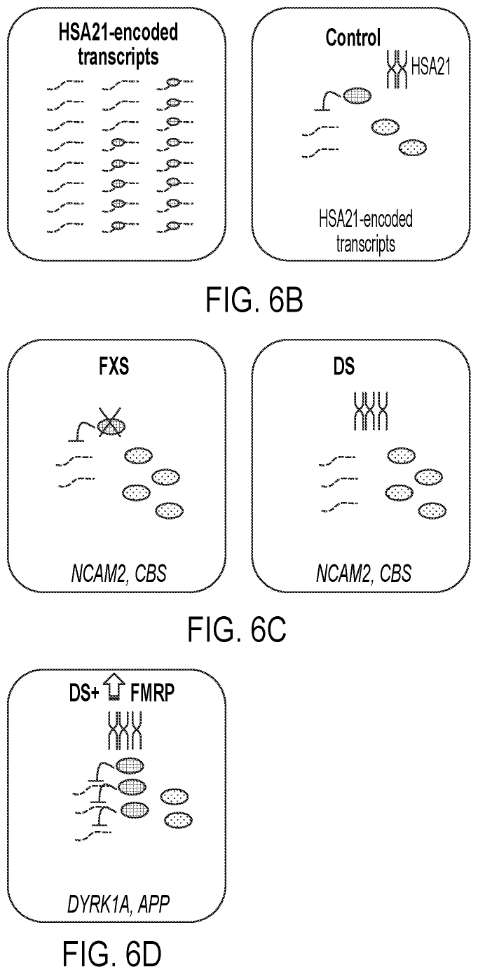

It is likely that there is no single mechanism of action for FMRP in a given cell type and cell state. The analyses presented herein highlight a more complex picture of gene regulation (FIGS. 6A-6D). For many targets, FMRP binding events were detected across multiple regions of a given transcript (i.e., both intronic and CDS regions). FMRP may bind a single transcript molecule in multiple regions at the same time or interact with different transcript molecules at different stages of processing. While FXS is canonically associated with increased protein expression and DS with increased gene dosage through an extra copy of a chromosome, it is reductionist to assume that all FMRP targets on HSA21 are coordinately upregulated in FXS and DS.

Without wishing to be bound by theory, in cases where FMRP targets are increased at the protein level in FXS but also bound as pre-mRNAs, it is speculated that FMRP may reduce the kinetics of post-transcriptional processing in the nucleus, and upon FMRP loss, transcripts are more readily processed and exported, leading to increased association with translational machinery. However, as discussed above, FMRP has previously been associated with diverse processes including transcriptional regulation, RNA splicing, and nuclear RNA export. Loss of FMRP followed by perturbation of any of these processes could impact downstream protein expression levels of individual targets. Even for fully mature mRNA targets, FMRP can phase-separate through its RGG domain and this sub-compartmentalization has been shown to impact deadenylation to inhibit translation in the cytoplasm, distinct from FMRP's canonical role in direct translational regulation. As FMRP localization and function likely change across developmental trajectories, and given that the analyses described herein are elucidating for early development, the analyses and studies described herein provide a basis for studies that may also elucidate FMRP's role in the developing versus the aging human brain.

While much remains to be learned about the mechanisms of action of FMRP for each individual RNA target, the model described herein allows for future therapeutic strategies aimed at DS that may be relevant for FXS, or vice versa, based on convergent target biology. For example, clinical trials have attempted to normalize DYRK1A with the goal of improving cognitive function in DS patients, and both DYRK1A and APP are strongly implicated in the development of AD in DS patients. Given that increasing FMRP from the endogenous locus alone was sufficient to reduce the expression levels of DYRK1A and APP in DS patient cells, methods to increase FMRP as potential therapeutic strategies in FXS could also have utility for DS patients or other patients with DYRK1A or APP over-expression.

Collectively, these data provide the first evidence for direct mechanistic overlap between FXS and DS and identify key genes at the interface of two of the most common genetic causes of intellectual disability and autism.

### Example 7: FMRP Upregulation is Sufficient to Downregulate the Expression of DS-Implicated Genes

To further assess the relevance of FMRP in the regulation of DS-implicated genes, studies were performed to test whether increasing FMRP dosage in the context of DS could potentially modulate target expression, given that many HSA21-encoded transcripts were upregulated in DS and also bound by FMRP. RNA-seq analyses of hPSCs and neurons using the isogenic DS patient and euploid control cell lines identified HSA21-encoded transcripts significantly upregulated in DS including DYRK1A, APP and BACE (FIGS. 7A-7D and FIGS. 7E and 7F and FIGS. 4D-4F), as expected based on the above protein level analyses. Cross-referencing RNA-seq data between FXS and DS cellular models revealed significant overlap in differential gene expression patterns, primarily in hPSCs (p=3.19×10′) and, to a lesser extent, in neurons (p=0.00495) where only a small number of genes were differentially expressed in FXS (FIGS. 4K, 4L, 4M). An inducible CRISPRa construct was stably introduced into the AAVS1 safe-harbor locus of a DS patient iPSC line and delivered a multiplexed piggyBac guide RNA (gRNA) vector containing three FMR1 activating gRNAs to facilitate acute and transient upregulation of endogenous FMRP (DS-CRISPRa; FIG. 7G).

Doxycycline induction of FMR1 in the DS-CRISPRa cell line led to efficient upregulation of FMRP expression at both 48 hrs (p=0.0210) and 120 hrs (p=0.0001), which returned to baseline after removal of doxycycline (“post-treatment”; FIG. 711). Importantly, inducing FMRP with CRISPRa had no impact on FXR1P expression levels, supporting the specificity of the CRISPRa system used herein (FIG. 7I). The effects of acute FMRP upregulation on DYRK1A, APP and BACE2 protein expression were queried (FIGS. 7J, 7K and 7L). Strikingly, acute upregulation of endogenous FMRP was sufficient to significantly reduce protein expression levels of DYRK1A in the DS-CRISPRa line after 120 hrs (p=0.0388), (FIG. 7J). Here, transient FMRP upregulation led to a sustained reduction in DYRK1A expression that persisted in the post-treatment (p=0.0182) (FIG. 7J). In addition, APP protein expression levels were probed following FMRP upregulation in the DS-CRISPRa cell line; APP expression levels were significantly reduced after 48 hrs (p=0.0387) and began to recover by 120 hrs (FIG. 7K). FMRP bound DYRK1A in both CDS and non-coding exon regions and APP in both CDS and intron regions (FIG. 7M and FIG. 7N), and could thus regulate these targets at transcriptional or translational levels. By contrast, acute upregulation of FMRP had no impact on BACE2 expression (FIG. 7L) where FMRP binding was only detected in introns (FIG. 7O). The experiments and data provide additional evidence for DYRK1A and APP target regulation by FMRP in an independent cell line using a complimentary approach. Importantly, the experiments and data described herein also provide proof-of-concept that transiently increasing endogenous FMRP in the context of DS is sufficient to significantly modulate key HSA21-encoded targets.

### Example 8: FMRP Upregulation is Sufficient to Reverse One-Fifth of the Global Transcriptional Perturbations in DS

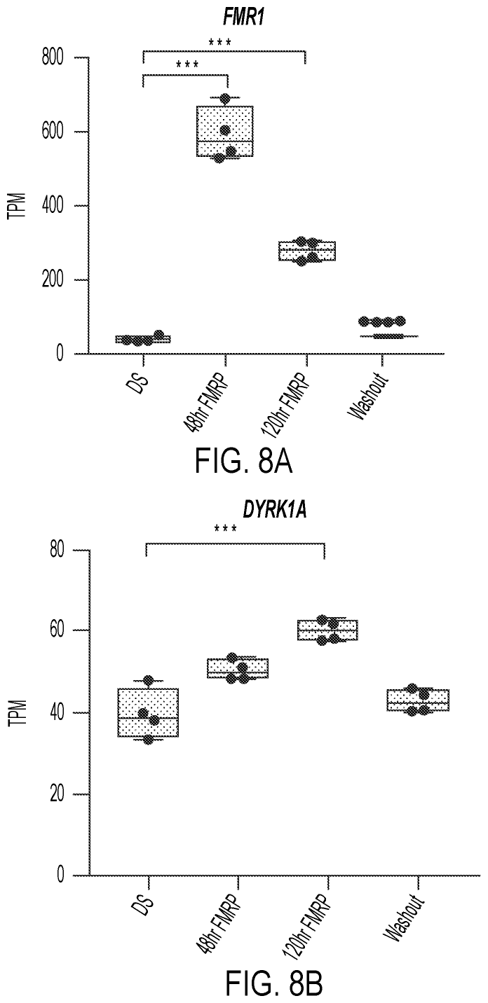

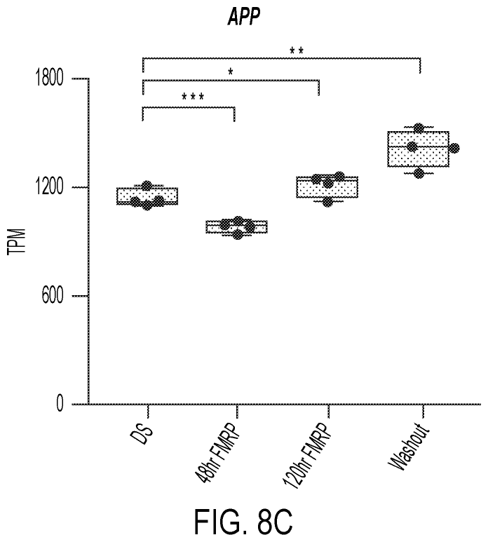

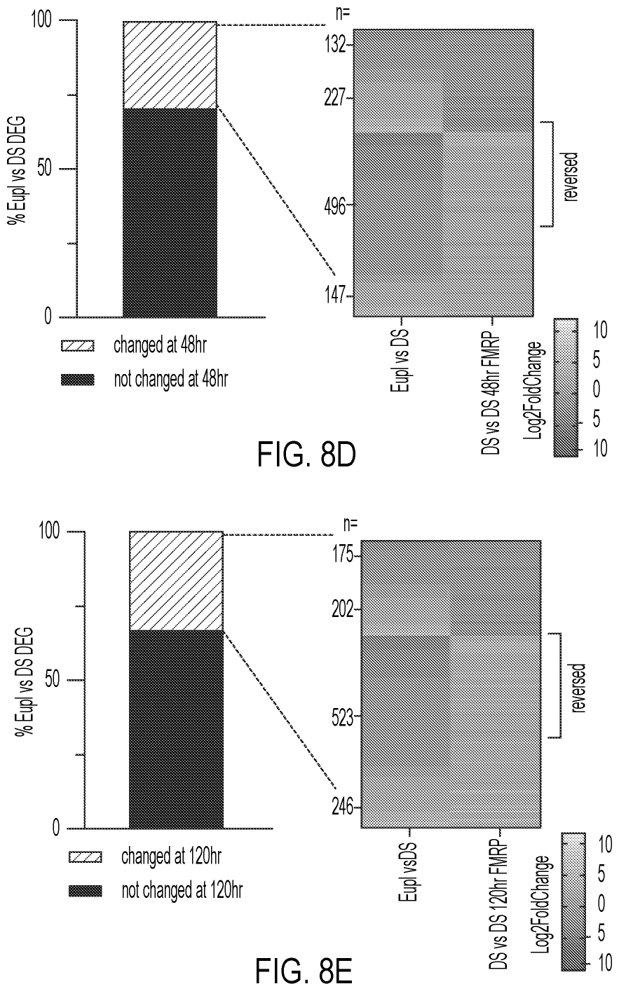

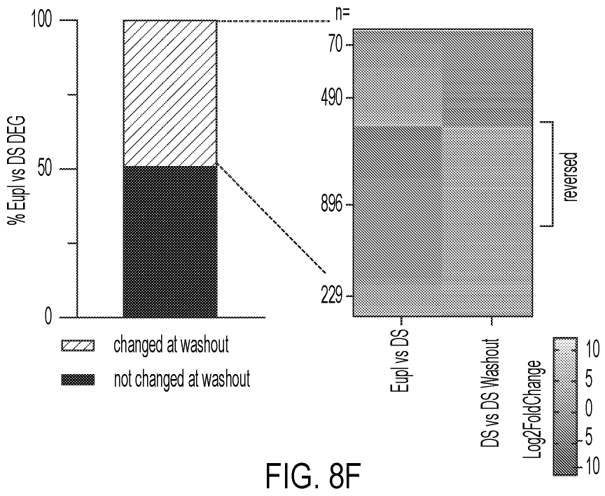

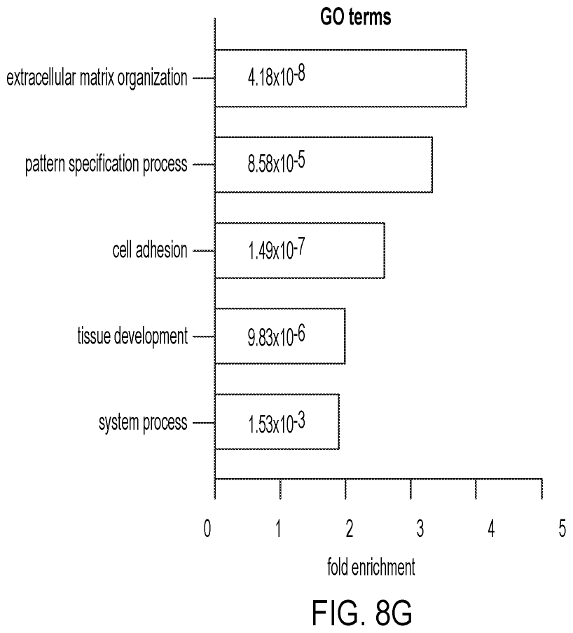

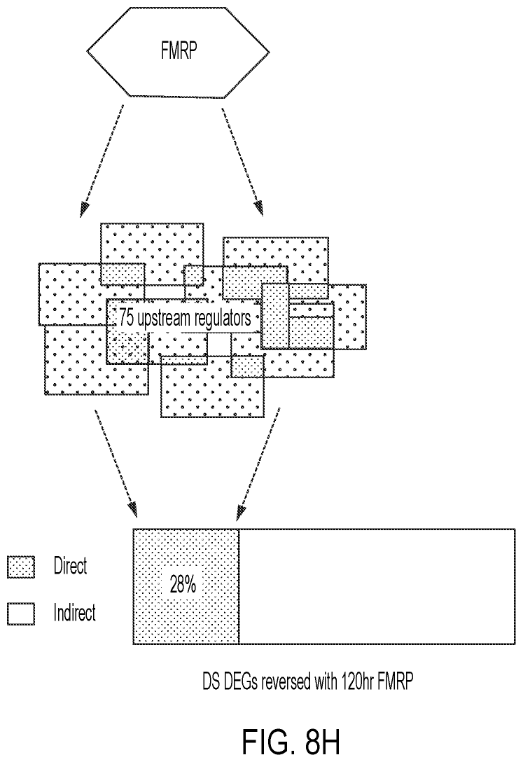

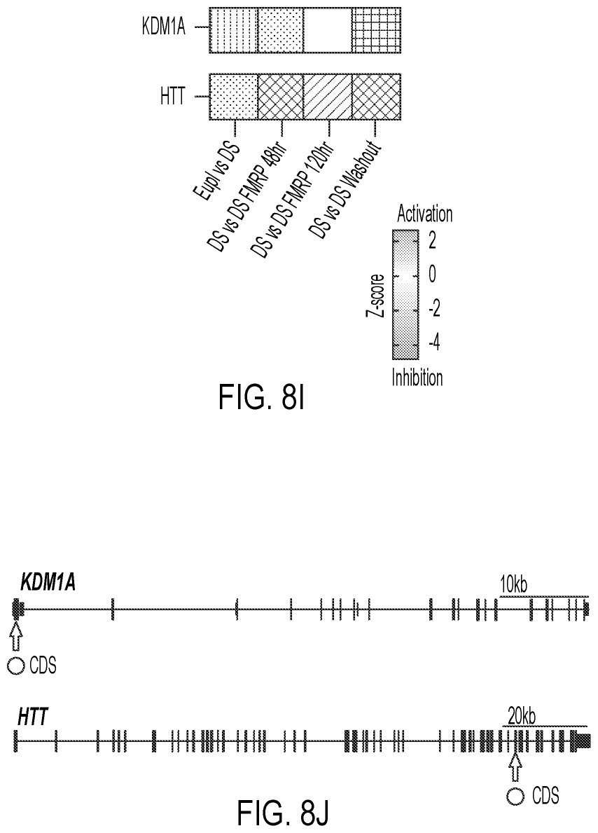

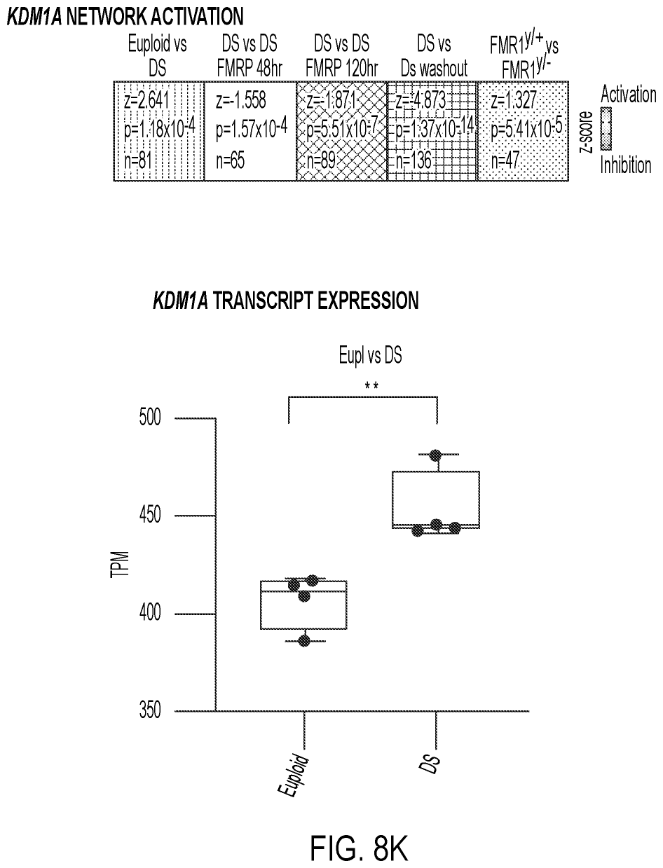

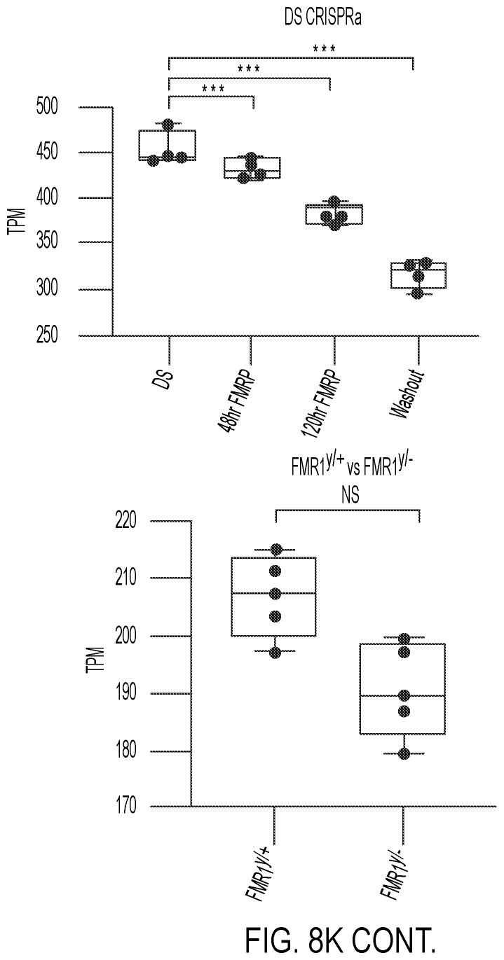

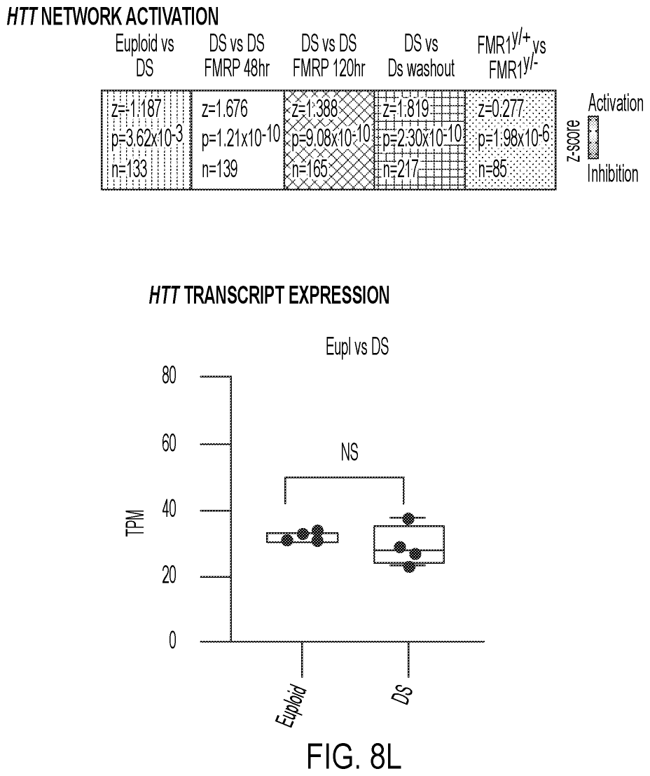

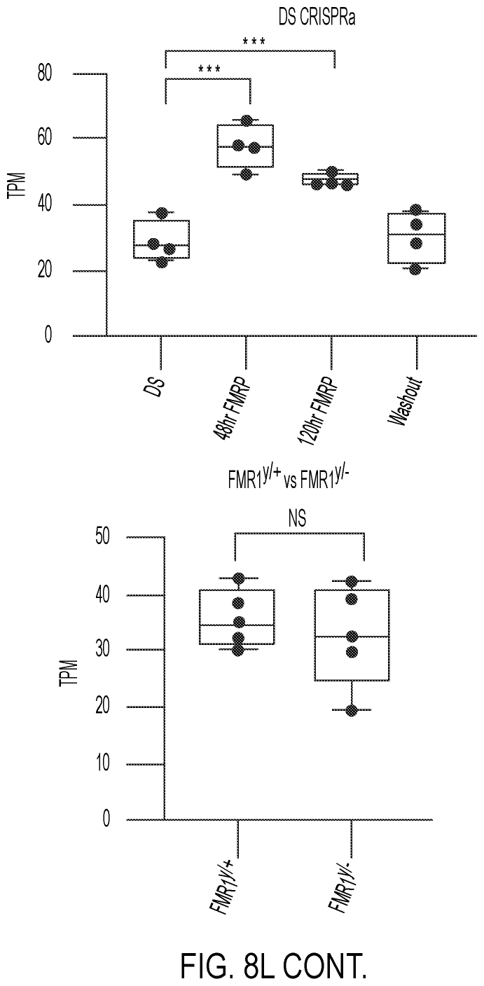

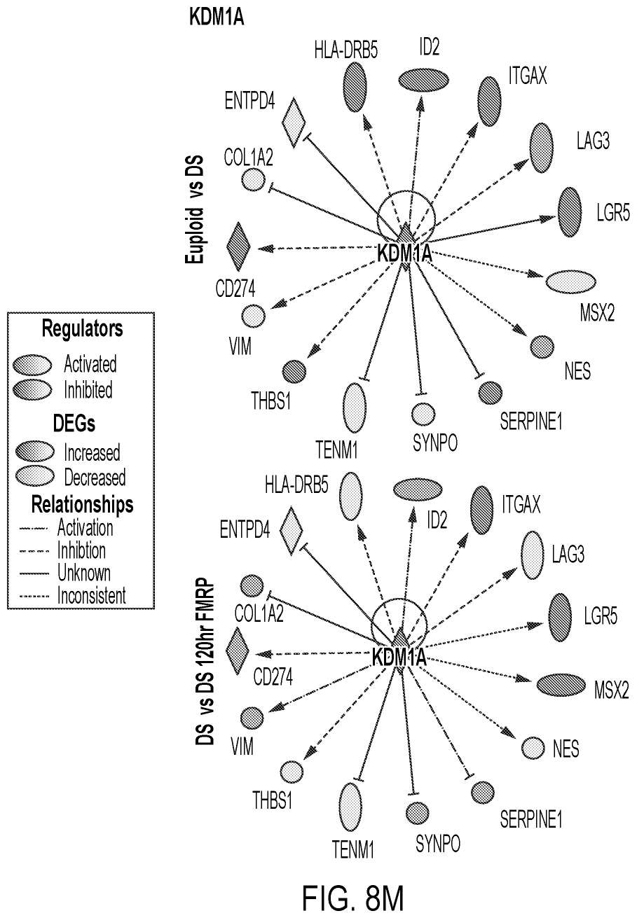

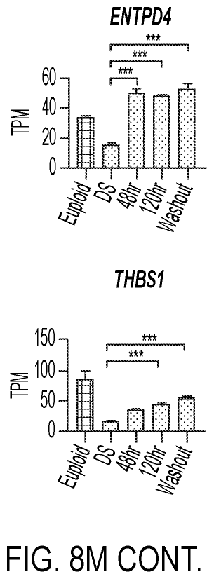

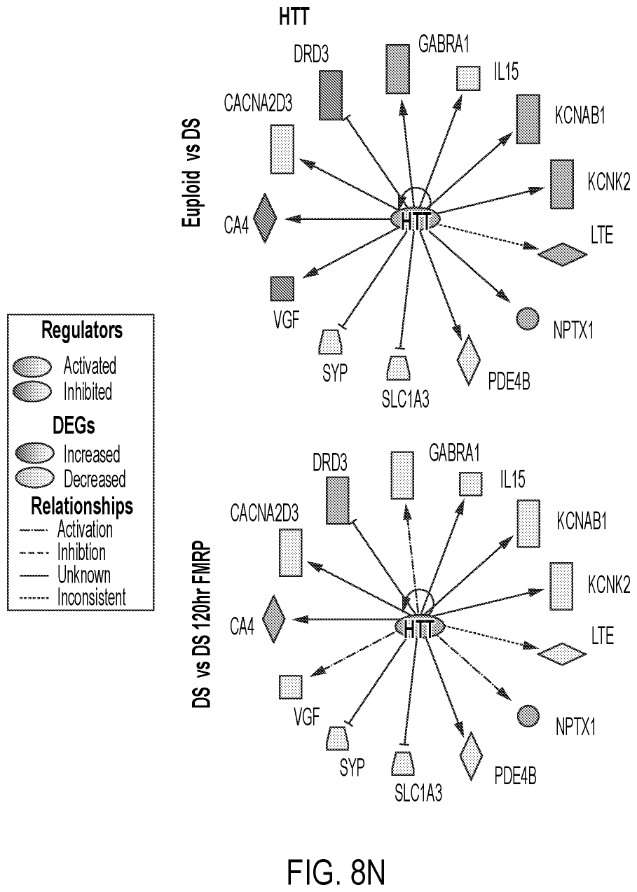

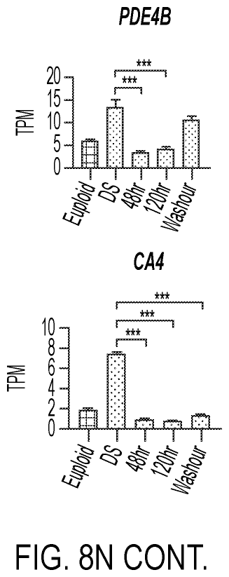

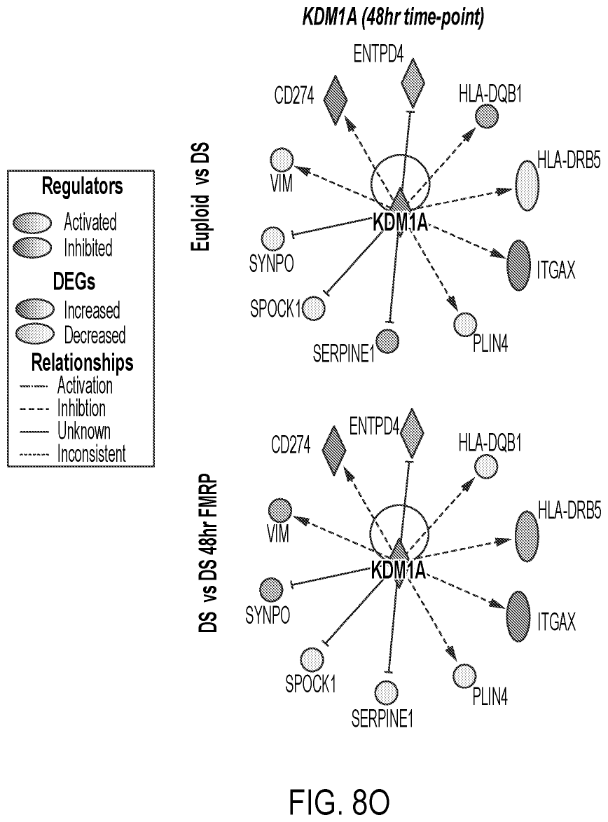

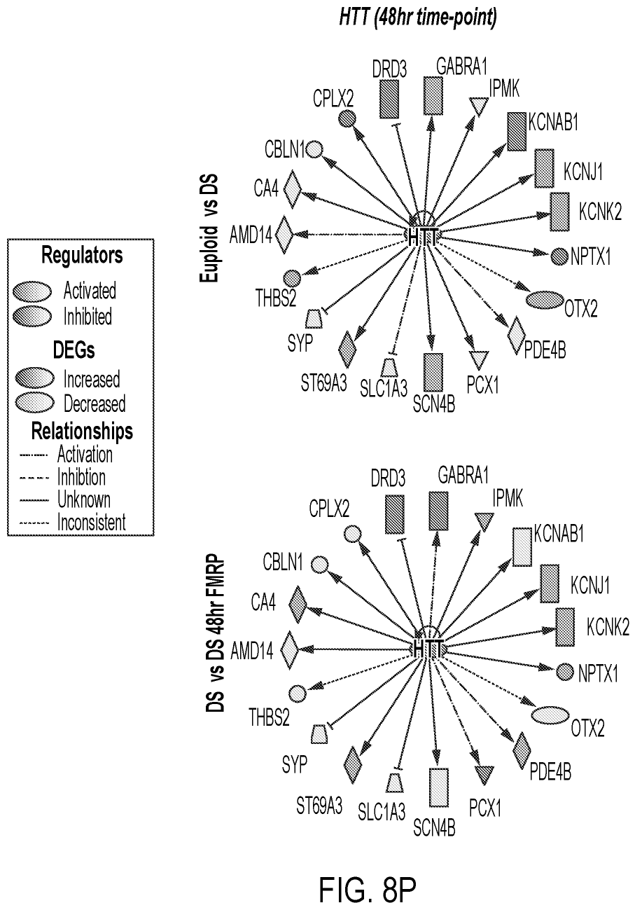

To identify additional gene and pathway perturbations in DS that could be modulated by FMRP upregulation, the impact on the global transcriptional landscape was assessed. For these analyses, the same isogenic cell lines and time-points used for protein level analyses in FIG. 7G were assessed using four replicates per condition with an adjusted p-value cutoff of 0.05 and loge fold change cutoff of 1. As expected, FMR1 transcript levels were significantly upregulated upon 48 hr and 120 hr FMRP CRISPRa induction and returned to baseline in the post-treatment condition (FIG. 8A). Evaluation of the other targets that showed significant protein changes upon FMRP CRISPRa showed that DYRK1A transcript levels were transiently increased at the 120 hr time-point (FIG. 8B), opposite the protein level changes (FIG. 7J), which could point to a compensatory increase in transcript abundance upon protein downregulation. APP transcript levels were transiently decreased by 48 hr FMRP CRISPRa induction (FIG. 8C), roughly paralleling the observed protein level changes (FIG. 7K). A total of 3450 significant DEGs in the DS CRISPRa (untreated) condition were identified compared to the isogenic euploid control (FIGS. 8D-8F). Strikingly, FMRP upregulation alone was sufficient to reverse the directionality of 21% of those DEGs at both 48 hr timepoint (FIG. 8D; 723/3450 DEGs) and 120 hr timepoint (FIG. 8E; 725/3450 DEGs), with a majority of changes persisting in the post-treatment condition (FIG. 8F; 1479/3450 DEGs). For example, 202 genes that were significantly upregulated in DS compared to euploid control were significantly downregulated in DS after 120 hrs of FMRP CRISPRa (p=2.68×10⁻¹³), and 523 genes that were significantly downregulated in DS compared to euploid control were significantly upregulated in DS after 120 hrs of FMRP CRISPRa (p=3.79×10⁻²⁸; FIG. 8E). In the post-treatment condition, 43% of all DEGs were reversed consistent with FMRP upregulation leading to both significant and sustained impacts on the global DS transcriptional program. Here, 521 genes that were significantly upregulated in DS compared to euploid control were significantly downregulated post-treatment (p=4.7×10¹⁴⁴) and 958 genes that were significantly downregulated in DS compared to euploid control were significantly upregulated post-treatment (p=7.7×10¹⁴¹), (FIG. 8F). GO analyses of DEGs significantly downregulated in DS that were then upregulated with FMRP induction at 120 hrs and post-treatment revealed terms such as “extracellular matrix organization” and “cell adhesion” indicating FMRP may impact these biological functions in DS (FIG. 8G).

To identify potential upstream regulators mediating the observed changes in gene expression following FMRP induction in DS, Ingenuity Pathway Analysis (IPA) was employed. Focusing on the 120 hr time point, 75 FMRP targets were identified that were predicted to function as upstream regulators, potentially accounting for up to 28% of DEGs that reversed directionality upon FMRP induction (FIG. 811). For example, FMRP bound the lysine 4 (K4) specific histone demethylase KDM1A/LSD1, associated with gene repression, in the CDS. KDM1A function was also identified as significantly activated in DS patient cells compared to euploid controls, and significantly inhibited at all time points following FMRP induction using IPA (FIG. 8I, FIG. 8J, and FIG. 8K). FMRP also bound the post-transcriptional regulator Huntingtin (HTT) in the CDS and HTT function was identified as being significantly inhibited in DS patient cells compared to euploid controls, and significantly activated at all timepoints following FMRP induction using IPA (FIGS. 8I, 8J and 8L). Examples of KDM1A and HTT gene networks that were specifically reversed upon 120 hr FMRP induction are shown in FIGS. 8M and 8N; networks from the 48 hr time-point are shown in FIGS. 80 and 8P. Note that some DS DEGs that reversed directionality upon FMRP induction such as Thrombospondin 1 (THBS1) persisted in the post-treatment condition (FIG. 8M) while other changes such as Phosphodiesterase 4B (PDE4B) reverted back to DS expression levels after post-treatment (FIG. 8N).

KDM1A plays diverse and essential roles in embryonic as well as neuronal and hematological cell types, where it promotes stemness/proliferation and prevents differentiation. Consistent with a profound role in development, de novo variants in KDM1A itself are associated with a rare intellectual disability syndrome. In addition to identifying novel KDM1A network activation in DS which could be reversed by FMRP induction, a modest but significant upregulation of KDM1A was also observed at the transcript level in DS compared to euploid control, which was downregulated by FMRP induction (FIG. 8K). Consistent with these findings, KDM1A function was also activated upon FMR1 loss in the FXS global transcriptional data obtained (FIG. 8K). Thus, these analyses identified KDM1A network activation in both DS and FXS, suggesting that FMRP may negatively regulate KDM1A expression. While the role of wildtype HTT is incompletely understood, it has been reported that the protein product huntingtin localizes to cytoplasmic P-bodies to suppress translation of its target RNAs. A significant difference in HTT transcript expression levels was not observed in DS compared to euploid control, but HTT was significantly upregulated upon FMRP induction (FIG. 8L). These results are consistent with an Fmr1-knockout mouse model which showed that FMRP positively regulates Htt transcript and protein expression (M. Shen et al., Nat Neurosci 22, 386-400 (2019)). Accordingly, novel HTT network inhibition was identified and shown in DS which could be reversed by FMRP induction, correlating with increased HTT expression. While fewer DEGs were detected in the FXS datasets as compared with the DS datasets, FMRP upregulation and loss had expected opposing effects on several KDM1A and HTT network genes.

Collectively, the described global transcriptional analyses revealed that upregulation of endogenous FMRP alone was sufficient to reverse the directionality of one-fifth of all significant DEGs in DS, thus strengthening the relevance of the molecular connection between DS and FXS. In addition, specific FMRP targets mediating some of the observed effects were identified, as exemplified by the novel network activation of KDM1A in DS and novel network inhibition of HTT in DS, both of which were partially reversed by FMRP induction.

The above examples, which describe results that define high-confidence FMRP targets in two physiologically relevant human cell types, support a pleiotropic role for FMRP in gene regulation. While a majority of mechanistic studies have focused on FMRP's role in translational regulation, the prevalence of intron binding events in both hPSCs and neurons highlights the need for further investigation of FMRP's role in additional aspects of RNA processing. Moreover, the examples herein have identified FXR1P as a key collaborator of FMRP for RNA binding generally, as well as intron binding specifically. It was surprisingly found through the experiments described herein that FXR1P was required for three-quarters of the FMRP binding events in neurons but appeared to be dispensable for many of FMRP's well-known synaptic targets. As many of these targets are reported to be locally regulated, the results are consistent with a model in which FXR1P is critical for the regulation of FMRP nuclear targets but potentially is not required for trafficking or translation of targets at the synapse. Not wishing to be bound by theory, while the results provide evidence for FMRP directly regulating RNA target expression or stability, this may not be a dominant mechanism in FXS. The described finding that a 10-fold larger proportion of FMRP targets were differentially expressed at the transcript level in hPSCs compared with neurons further suggests that the prevalence of certain mechanisms of gene regulation may vary across cell types or cell states, underscoring the relevance of investigation in diverse cellular contexts.

Importantly, existing knowledge of molecular mechanisms underlying FXS has failed to translate into effective therapeutic strategies, and target prioritization for diseases that impact RNA binding proteins (RBPs) remains a substantial challenge. For this reason, FMRP targets were cross-referenced in hPSCs and neurons with existing neurodevelopmental disease gene datasets, which revealed significant enrichment for genes implicated in autism and developmental disorders, but not epilepsies, in both cell types. While enrichment patterns for neuronal targets were not unexpected based on previous mouse brain datasets, overlap with pluripotent stem cells suggests there could be gene perturbations in these diseases impacting early embryonic development. Unexpectedly, novel gene set overlap was discovered between DS and FXS, and the described experimental analyses provide the first evidence that overlap between FMRP targets and another neurodevelopmental disease can translate into specific shared molecular perturbations. First, knowledge of DS disease biology was leveraged to prioritize and identify specific targets that were upregulated in both DS and FXS. The CBS and NCAM2 genes are high-priority targets for functional characterization in FXS. Highlighted by the studies described herein is the need to similarly assess points of concordance between FXS and other neurodevelopmental diseases such as autism or schizophrenia, as gene set overlap did not always translate into coordinate molecular perturbations based on the analyses. Not wishing to be bound by theory, while many gene targets and patient phenotypes do not overlap between DS and FXS, individual genes disrupted in both diseases may be more likely to underlie shared phenotypes which include intellectual disability, deficits in expressive communication, as well as increased rates of autism, seizure disorders and mental health disorders compared with the general population. The consistent upregulation of some proteins like CBS across genetic backgrounds may indicate their involvement in more penetrant phenotypes, compared with targets like NCAM2 whose expression levels varied with genetic background and could therefore contribute to more variable traits known to exist in both DS and FXS. With regard to potential mechanisms of NCAM2 gene regulation, it has been reported that FMRP plays a role in RNA editing using adult post-mortem brain tissue to find a significant decrease in A-to-I RNA editing of NCAM2 in FXS patients compared with controls (S. S. Tran et al., Nat Neurosci 22, 25-36 (2019)). As described herein, FMRP binding was found in the same intron of NCAM2 in neurons in vitro that showed differential A-to-I editing in the FXS adult postmortem brain tissue. This mechanism of NCAM2 regulation may influence transcript abundance through nuclear retention, miRNA-based mechanisms or altered stability.

Based on the discoveries and results described herein, a single RBP has the potential to regulate directly and indirectly a large number of DS-implicated genes in trans. Of note, the CRISPRa experiments described in the above Example confirmed that FMRP could directly modulate expression of the key HSA21-encoded targets DYRK1A and APP. FMRP bound both CDS and intron or non-coding exon regions of each gene, suggesting that FMRP can engage in transcriptional processing of these targets, separate from, or in addition to, translational regulation. While little is known about FMRP regulation of DYRK1A, it has been reported that FMRP can translationally repress APP (C. J. Westmark, J. S. Malter, PLoS Biol 5, e52 (2007)). Without wishing to be bound by theory, the reduction in APP and DYRK1A that is found following FMRP upregulation in DS may be due to enhanced translational repression. This model is also consistent with a lack of observed modulation of BACE2, which was bound by FMRP exclusively in introns. For many targets, FMRP binding events were detected across multiple regions of a given transcript; thus, FMRP may bind a single transcript molecule in multiple regions at the same time, or interact with different transcript molecules at different stages of processing.

The experimental results described supra indicate that, in addition to impacting individual HSA21-encoded targets, the upregulation of FMRP alone was also sufficient to reverse the directionality of over 40% of the global transcriptional perturbations in DS. The experiments identified upstream regulators in DS and cross-referenced those data with FMRP targets to discover and identify that multiple gene networks, including KDM1A and HTT, were perturbed in DS and were partially reversed by FMRP induction. The results indicated that KDM1A function is activated in DS and FXS, and that KDM1A expression and function is inhibited by FMRP induction in DS, consistent with FMRP negatively regulating KDM1A. The use of KDM1A inhibitors has been reported in mouse models of Kabuki syndrome (L. Zhang et al., bioRxiv, (2020)) and aging (T. Maes et al., PLoS One 15, e0233468 (2020)) to improve memory, as well as in clinical trials of cancer (T. Maes et al., Cancer Cell 33, 495-511 e412 (2018)). The experimental results described supra also suggest that HTT function is inhibited in DS and that HTT expression and function can be enhanced by FMRP induction in DS. These results are consistent with data from a mouse model indicating that FMRP positively regulates HTT expression (M. Shen et al., Nat Neurosci 22, 386-400 (2019)), and it is possible that both DS and FXS share downregulated HTT function. The present invention advantageously provides a therapeutic and method related to KDM1A inhibition and HTT upregulation in connection with treatment of both DS and FXS. While KDM1A and HTT were not immediately apparent for study in DS given that neither gene is localized to HSA21, but their potential mis-regulation in both DS and FXS makes them compelling candidates for further interrogation and as targets for therapeutic intervention in these diseases/disorders. Such convergent genes and pathways as described herein pave the way for other biological discoveries and therapeutic strategies aimed at DS as being relevant for FXS, and vice versa. It is also possible that some of the gene and pathway perturbations described and identified herein as being shared between DS and FXS have broader relevance for neurodevelopmental diseases.

Without wishing to be bound by theory, it is highly likely that HSA21-encoded FMRP targets, like other FMRP targets, are bound through a combination of mechanisms rather than a single transcript feature being enriched on this chromosome. Additional analyses of RNA modifications such as m6A and A-I editing previously associated with FMRP binding in other cell types (S. S. Tran et al., Nat Neurosci 22, 25-36 (2019); P. J. Hsu et al., J Biol Chem, (2019)), or protein co-factors in addition to FXR1P, may assist in further parsing how individual HSA21-encoded targets are recognized and ultimately regulated. As the described examples and studies highlight, no single mechanism of action may be attributed to FMRP in a given cell type and cell state. While an unexpected pattern of HSA21-enrichment was found in two human cell types as described herein, HSA21-encoded targets need not be enriched compared to other chromosomes in order to support a connection between DS and FXS. Previous meta-analyses of mouse targets specifically identified high-priority DS genes enriched among the direct targets of FMRP in the absence of a general chromosome enrichment pattern.

FMRP targets can be defined in additional human cell types to understand not only connections to DS but also to further dissect the contributions of individual cell types to disease phenotypes. FMRP target analyses in hippocampal CA1 pyramidal versus cerebellar granule neurons from adult mouse brain support the notion that cell-type specific FMRP targets can contribute to different aspects of disease pathology (K. Sawicka et al., Elife 8, (2019)). Given that imbalances in excitation-inhibition are thought to contribute to many neurodevelopmental diseases including autism, FXS and DS, combined with recent exome sequencing studies and single-cell RNA-seq data from the developing human cortex indicating that known autism risk genes are enriched for expression in both excitatory neurons and inhibitory interneurons during development, FMRP target analyses in inhibitory neurons in particular may identify additional relevant cell type-specific genes and pathways. Of technical note, the neuron input amount required for eCLIP-seq in the studies described supra was challenging to achieve and required differentiating hundreds of millions of neurons. Other FMRP target studies utilizing in vitro derived human cell types may require protocols compatible with lower cell input amount or extremely high-yield differentiation paradigms. In addition, stringent thresholding criteria were applied to all of the eCLIP-seq analyses described herein, favoring false negatives over false positives. However, this strategy may exclude additional, bona fide FMRP targets. For example, FMRP has been reported to bind to and regulate APP in mouse neurons (C. J. Westmark, J. S. Malter, PLoS Biol 5, e52 (2007)). In the datasets obtained and analyzed herein, significant FMRP binding events in APP were detected in hPSCs; in neurons, the strongest binding event had a −log₁₀ p-value of 2.516, which fell below the −log₁₀ p-value cutoff of 3. Moreover, the experimental studies and analyses described herein are highly relevant for embryonic development.

The findings and results described and exemplified herein provide new insights into FMRP function in human stem cells and neurons, uncover a novel molecular connection between DS and FXS and identify key genes and pathways at the interface of two of the most common genetic causes of intellectual disability and autism.

### Example 9: Materials and Methods of the Examples

The materials and methods described herein relate to the above Examples and the results obtained from the described experiments, studies and analyses.

Stem cell resources and culture. The XY human embryonic stem cell line H1 was commercially obtained from WiCell Research Institute and was used to generate isogenic FMR1^{y/+} and FMR1^{y/-} cell lines (S. G. Susco et al., Dev Biol 468, 93-100 (2020)), as well as FXR1^{-/-} cell lines using CRISPR-Cas9. The XY human DS patient iPSC lines UWWC1-DS1, UWWC1-2D53 and the euploid control UWWC1-DS2U (isogenic with UWWC1-DS1) were commercially obtained from WiCell Research Institute (J. P. Weick et al., Proceedings of the National Academy of Sciences of the United States of America 110, 9962-9967 (2013)), referred to as DS iPSC A, DS iPSC B and control iPSC B, respectively, in the studies described herein. The control iPSC line CW60278, referred to as control iPSC A, was obtained from the California Institute for Regenerative Medicine iPSC repository (Fujifilm, Cellular Dynamics). Three FXS patient iPSCs were reprogrammed at the Harvard Stem Cell Institute Core (Cambridge Mass.) with Sendai virus using XY patient fibroblasts. The following fibroblast cell lines were obtained from the NIGMS Human Genetic Cell Repository at the Coriell Institute for Medical Research: GM05131, GM04026 and GM09497, referred to as FXS iPSC A, FXS iPSC B and FXS iPSC C in the described study, respectively, after reprogramming. XY cell lines were selected based on clinical data indicating that males are typically more severely affected by FXS than females and to avoid heterogeneity with respect to X-chromosome inactivation in edited clones. All studies using hESCs/iPSCs followed institutional IRB and ESCRO guidelines approved by Harvard University. Cell culture was carried out as previously described (Hazelbaker, D. Z. et al., Stem cell reports 9, 1315-1327 (2017); Hazelbaker, D. Z. et al., bioRxiv (2019); Bara, A. M. et al., Stem Cell Res 17, 441-443 (2016)). In brief, stem cells were grown and maintained in mTeSR medium (Stem Cell Technologies) on geltrex-coated (Life Technologies) plates. All cell lines underwent QC testing to confirm expected karyotypes and genotypes, absence of mycoplasma, expression of pluripotency markers and tri-lineage potential. SNP genotyping was performed using the Infinium PsychArray (Illumina) and chromosomal alterations were evaluated using the MoChA caller (Mattioli, F. et al., bioRxiv (2019)). G-band karyotyping analysis was performed by Cell Line Genetics. Replicates for experiments using hPSCs refer to separate wells or plates of a given hPSC line.

CRISPR-Cas9 based genome engineering. CRISPR-Cas9 based genome engineering experiments were carried out as previously described (Hazelbaker, D. Z. et al., Stem cell reports 9, 1315-1327 (2017); Hazelbaker, D. Z. et al., bioRxiv (2019); Bara, A. M. et al., Stem Cell Res 17, 441-443 (2016)). In brief, to generate FXR1^{-/-} cells, the hPSC line H1 was transfected with Cas9 nuclease plus an FXR1 gRNA targeting exon 8 of the full-length gene upstream of predicted functional domains (AGCTCAATGGCGGTAACTCC), using the NEON system (Life Technologies) followed by clonal isolation and screening. To generate CRISPRa cell lines, TRE-dCas9-VPR-eGFP was inserted into the AAVS1 locus of the DS patient iPSC A (UWWC1-DS1) using TALENs, as previously described (Hazelbaker, D. Z. et al., Scientific reports 10, 635 (2020)). Three gRNAs targeting FMR1 for CRISPRa (g1: GCGCTGCTGGGAACCGGCCG, g2: CAGGTCGCACTGCCTCGCGA, g3: AGACCAGACACCCCCTCCCG) were designed with the CRISPR-ERA tool (Liu, H. et al. Bioinformatics 31, 3676-3678 (2015), cloned into a multiplexed piggyBac vector and co-transfected in the presence of a piggyBac transposase, as previously described (Hazelbaker, D. Z. et al., Scientific reports 10, 635 (2020)). Following selection with G418 and blasticidin, cells were assessed for EGFP+/mRFP+ fluorescence and FMRP expression following doxycycline induction.

Generation of human excitatory neurons. Human neurons were generated as previously described (Zhang, Y. et al., Neuron 78, 785-798 (2013); Nehme, R. et al., Cell reports 23, 2509-2523 (2018)). In brief, hPSCs were transduced with TetO-Ngn2-T2A-Puro and Ubiq-rtTA lentivirus or TetO-Ngn2-P2A-Zeo and CAG-rtTA were integrated into the AAVS1 safe-harbor locus using TALENs. Cells were then treated with doxycycline to induce ectopic Ngn2 expression combined with the extrinsic addition of SMAD inhibitors (SB431542, 1614, Tocris, and LDN-193189, 04-0074, Stemgent), Wnt inhibitors (XAV939, 04-00046, Stemgent) and neurotrophins (BDNF, GDNF, CNTF) followed by puromycin treatment to eliminate uninfected stem cells and maintenance in Neurobasal medium. Ultra-high lentiviral titer was generated by Alstem, LLC. Alternatively, TetO-Ngn2-P2A-Zeo and CAG-rtTA were integrated into the AAVS1 safe-harbor locus using TALENs. Replicates for experiments using neurons refer to independent neuronal differentiations from a given hPSC line.

eCLIP-seq and analyses. eCLIP was performed as previously described (Van Nostrand, E. L. et al., Nature methods 13, 508-514 (2016)). In brief, FMR1^{y/+} and FMR1^{y/-} hPSCs or FMR1^{y/+} and FMR1^{y/-} neurons at day 14 of in vitro differentiation were UV-crosslinked, lysed, sonicated and treated with RNAse I. 2% of each lysate sample was used to generate a parallel size-matched input (SMInput) library. The remaining lysates were used for immunoprecipitation (IP) with the anti-FMRP antibodies RN016P (MBL International) and ab17722 (Abcam). Bound RNA fragments in the IPs were dephosphorylated and 3′-end ligated to an RNA adapter. Complexes from SMInputs and IPs were run on an SDS-PAGE gel and transferred to a nitrocellulose membrane. Membranes were excised, and proteinase K treated to release RNA from protein complexes. Samples were then dephosphorylated and 3′-end ligated to an RNA adaptor. All IP and SMInput samples were reverse transcribed using AffinityScript (Agilent). cDNAs were 5′-end ligated to a DNA adaptor. cDNA yields were quantified by qPCR, and 100-500 fmol of libraries were generated using Q5 PCR mix (New England Biolabs). Reads were trimmed to remove adapters, and mapped to human repetitive elements from RepBase (version 18.05) by STAR. The remaining reads that were not mapped to repetitive elements were mapped to the human genome assembly hg19 by STAR. PCR duplicates were removed using the unique molecular identifier (UMI) sequences. Peaks were assigned by CLIPper, and annotated to gene regions in Gencode (v19) with the following priority order: CDS, 5′UTR, 3′UTR, proximal intron, and distal intron. Distal intron regions were defined as the intronic regions more than 500 bp from an exon-intron junction(distintron500), and proximal intron regions were defined as the intronic regions up to 500 bp from an exon-intron junction (proxintron500). The peak fold changes were calculated by normalizing usable reads between immunoprecipitation and the SMInput with a peak threshold of at least 8-fold enrichment in IP over the SMInput and p-values <10⁻³. Enriched p-values were calculated by Chi-square test, or Fisher's exact test if the usable read number in IP or SMInput was below 5. The above eCLIP-seq analysis pipeline was also performed using FMR1^{y/-} hPSCs (RN016P) and FMR1^{y/-} neurons (RN016P and ab17722). Significant peaks in the FMR1^{y/+} dataset were removed from analysis if an overlapping significant peak also occurred in the corresponding FMR1^{y/-} dataset.

### Gene Ontology (GO) Analyses

GO analyses of eCLIP-seq data were performed using the Panther overrepresentation test and GO database annotation with Fisher test and Bonferroni correction. To determine the size of the RNA universe in both hPSCs and neurons, the TPM counts from the RNA-seq data were assessed. A gene was counted as expressed if it had an average TPM≥1 across five replicates in each FMR1^{y/+} cell type. This generated 15,316 RNAs expressed in neurons, and 14,233 RNAs expressed in hPSCs. These universes were then used to establish the background for GO analysis.

RNA motif and structure analyses. To analyze potential motif enrichment in FMRP binding sites, HOMER analyses were processed as described in CLIP_analysis_legacy (https://github.com/YeoLab/clip analysis legacy). Briefly, HOMER was used to identify de novo motifs by comparing significant enriched peaks with randomly defined peaks. The command was ‘findMotifs.p1<foreground>hg19<output location>-rna -S 20 -len 6 -p 4 -bg<background>’. Foreground was a bed file of significant enriched peaks; the background was randomly defined peaks within the same annotated region as foreground peaks. To analyze potential structure enrichment in FMRP binding sites, base pairing probabilities were calculated in and around FMRP binding sites detected by eCLIP-seq. FMRP binding sites were split into length categories of ≤50, or ≤200. Binding sites in each category were extended symmetrically in size to encompass either exactly 50 bases, or exactly 200 bases. The equally sized portions of sequence then underwent computational structure prediction with RNAfold 2.0, using the command “RNAfold -p” to calculate pair probabilities of every base in the sequence. Pair probabilities were summed for each base to give the final probability of a base being paired. Base pairing probabilities were also predicted for equally sized regions flanking the 50-base or 200-base FMRP binding sites. Base pairing probabilities were averaged for bases in flanking or FMRP region, which were each divided in two. Averages were plotted as violin plots with Seaborn 0.9.0.

RNA-seq of FXS and DS Cell Lines. RNA was extracted from hPSCs and neurons using the AllPrep DNA/RNA/miRNA Universal Kit (Qiagen) using five biological replicates for each genotype and cell type (e.g., five separate wells of hPSCs or five independent batches of neuronal differentiations). Sequencing libraries were prepared using the Illumina TruSeq HS Stranded Total RNA kit with Ribo-Zero Gold for rRNA depletion and quantified using the Agilent Bioanalyzer RNA Pico kit. Libraries were sequenced on a HiSeq 2500 at the Broad Institute Genomics Platform to generate 100 bp paired end reads. RNA-seq QC and analysis was performed by the Harvard Chan Bioinformatics Core, Harvard T. H. Chan School of Public Health, Boston, Mass. Reads were processed to counts through the bcbio RNA-seq pipeline implemented in the bcbio-nextgen project (https://bcbio-nextgen.readthedocs.org/en/latest/). Raw reads were examined for quality issues using FastQC (http://www.bioinformatics.babraham.ac.uk/projects/fastqc/) to ensure library generation and sequencing were suitable for further analysis. As necessary, adapter sequences, other contaminant sequences, such as polyA tails and low quality sequences with PHRED quality scores less than five, were trimmed from reads using cutadapt (M. Martin, Cutadapt removes adapter sequences from high-throughput sequencing reads. 17, 3 (2011)). Trimmed reads were aligned to Ensembl build GRCh38_90 of the Homo sapiens genome (human), using STAR (A. Dobin et al., STAR: ultrafast universal RNA-seq aligner. Bioinformatics (Oxford, England) 29, 15-21 (2013)). Alignments were checked for evenness of coverage, rRNA content, genomic context of alignments (for example, alignments in known transcripts and introns), complexity and other quality checks using a combination of FastQC, Qualimap (F. Garcia-Alcalde et al., Qualimap: evaluating next-generation sequencing alignment data. Bioinformatics 28, 2678-2679 (2012)), MultiQC (https://github.com/ewels/MultiQC) and custom tools. Counts of reads aligning to known genes were generated by featureCounts (Y. Liao, G. K. Smyth, W. Shi, featureCounts: an efficient general purpose program for assigning sequence reads to genomic features. Bioinformatics 30, 923-930 (2014)). In parallel, TPM measurements per isoform were generated by quasialignment using Salmon (R. Patro, G. Duggal, C. Kingsford, Accurate, fast, and model-aware transcript expression quantification with Salmon. bioRxiv, (2015)). Differential expression at the gene level was called with DESeq2 (M. I. Love, W. Huber, S. Anders, Moderated estimation of fold change and dispersion for RNA-seq data with DESeq2. Genome Biol 15, 550 (2014)), preferring to use counts per gene estimated from the Salmon quasialignments by tximport (C. Soneson, M. I. Love, M. D. Robinson, Differential analyses for RNA-seq: transcript-level estimates improve gene-level inferences. F1000Res 4, 1521 (2015)). Quantifying at the isoform level has been shown to produce more accurate results at the gene level.

RNA-seq of CRISPRa Cell Lines

RNA was extracted from hPSCs using the AllPrep DNA/RNA/miRNA Universal Kit (Qiagen) using four replicates (e.g., four separate wells of hPSCs) for each condition: Euploid Control iPSC B, DS CRISPRa (untreated), DS 48 hr FMRP CRISPRa, DS 120 hr FMRP CRISPRa and DS 120 hr on/120 hr off FMRP CRISPRa. Libraries were prepared using Roche Kapa mRNA HyperPrep strand specific sample preparation kits from 200 ng of purified total RNA according to the manufacturer's protocol on a Beckman Coulter Biomek i7. The finished dsDNA libraries were quantified by Qubit fluorometer and Agilent TapeStation 4200. Uniquely dual indexed libraries were pooled in equimolar ratio and shallowly sequenced on an Illumina MiSeq to further evaluate library quality and pooling balance. The final pool was sequenced on an Illumina NovaSeq 6000 targeting 30 million 100 bp read pairs per library. Sequenced reads were aligned to the UCSC hg19 reference genome assembly and gene counts were quantified using STAR (v2.7.3a) (A. Dobin et al., STAR: ultrafast universal RNA-seq aligner. Bioinformatics (Oxford, England) 29, 15-21 (2013)). Differential gene expression testing was performed by DESeq2 (v1.22.1) (M. I. Love, W. Huber, S. Anders, Moderated estimation of fold change and dispersion for RNA-seq data with DESeq2. Genome Biol 15, 550 (2014)). RNAseq analysis was performed using the VIPER snakemake pipeline (M. Cornwell et al., VIPER: Visualization Pipeline for RNA-seq, a Snakemake workflow for efficient and complete RNA-seq analysis. BMC Bioinformatics 19, 135 (2018)). Library preparation, Illumina sequencing and VIPER workflow were performed by the Dana-Farber Cancer Institute Molecular Biology Core Facilities and we appreciate the analytical support from Zach Herbert. Ingenuity Pathway Analysis (IPA) was used for analyses of upstream regulators.

Western blot analysis. Cells were lysed using RIPA lysis buffer (Life Technologies) with protease inhibitors (cOmplete, Mini, EDTA-free Protease Inhibitor Cocktail, Roche). 20 μg of protein as determined by Peirce BCA Protein Assay kit (Thermo Scientific) was loaded onto Bolt 4-12% Bis-Tris Plus gels (Invitrogen), transferred using the iBlot2 system (Thermo Scientific), blocked in 5% milk in TBST, and then incubated with primary antibodies in 1% milk in TBST overnight at 4° C. Membranes were rinsed in TBST, incubated with secondary antibodies for 1 hour at room temperature, rinsed in TBST, and then developed using SuperSignal WestFemto Maximum Sensitivity Substrate (Thermo Scientific). The following primary antibodies were used: anti-FMRP (Abcam ab17722), anti-GAPDH (EMD MAB374), anti-ADARB1 (Thermo Scientific PAS-34828), anti-NCAM2 (Abcam ab173297), anti-DYRK1A (Bethyl A303-802A), anti-FXR1P (ML13 courtesy E. Khandjian), anti-PCP4 (Abcam ab197377), anti-CBS (Proteintech 14787-1-AP), anti-APP (Abcam ab32136), anti-BACE2 (Abcam ab270458). For quantification, bands were analyzed in FIJI, normalized to GAPDH, averaged, and plotted with SEM for error bars. All Western blots were performed on triplicate samples and significance was calculated by unpaired two-tailed t-test for comparisons between two groups. Prism (GraphPad Software) was used for statistical analyses.

IP-Western Blot. For FMRP IP-Western blot analysis, neurons were lysed in Pierce IP lysis buffer (Life Technologies) and quantified with Peirce BCA Protein Assay kit (Thermo Scientific). 1 mg of protein lysate was incubated with 1 μg of antibody overnight with rotating at 4° C. Protein G magnetic beads (Pierce) were washed twice in cold lysis buffer and added at a concentration of 0.437 mg of beads to 1 μg of antibody per IP and incubated at 4° C. with rotating for 4 hours. Samples were placed on the Dynamag2 (Invitrogen), and the supernatant sample was collected. Samples were then washed once with cold lysis buffer (wash 1), and twice with cold PBS (washes 2 and 3). The washed beads were then resuspended, and equal fractions were boiled in 4× Laemmli buffer with BME for 5 minutes, placed on the magnet, and loaded into Bolt 4-12% Bis-Tris Plus gels (Invitrogen) for Western blotting as described above. FMR1^{y/+} neurons were immunoprecipitated with anti-FMRP (Abeam ab17722) and anti-IgG (Sigma 12370) and FMR1^{y/-} neurons were immunoprecipitated with anti-FMRP (Abeam ab17722). All three immunoprecipitations were blotted for FXR1P with anti-FXR1P ML13 (courtesy E. Khandjian).

ENCODE Dataset Meta-analyses. To analyze data across chromosomes for 120 RNA binding protein (RBPs) from K562 cells collected with eCLIP-seq as part of the ENCODE Consortium Project, the number of replicable binding sites was counted on each chromosome and normalized to the number of transcribed and mappable bases on the given chromosome (based on Hg19 NCBI transcript annotations and ENCODE blacklist data). Counts were also normalized by the total number of replicable binding sites for a given RBP, and scaled by a constant for viewing. Heat maps were plotted and RBPs hierarchically clustered using Seaborn 0.9.0.

### Comparative Analyses

To compare the human targets with published datasets, 842 mouse FMRP targets previously identified by Darnell et al., (J. C. Darnell et al., Cell 146, 247-261 (2011)) were utilized; the corresponding 865 human homologs were identified using Ensembl Biomart. The ‘high’ and ‘stringent’ mouse FMRP CA1 targets from Sawicka et al., (K. Sawicka et al., Elife 8, (2019)) were also used, resulting in 1266 targets after converting from mouse to human homologs using Ensembl Biomart. 3322 targets were identified from a K562 FMRP eCLIP-seq dataset from the ENCODE project. For epilepsy comparisons, the list of top 200 genes with burden of deleterious ultra-rare variants with an allele count <3 in all epilepsy cases (Epi25, 2019) were used. For statistical tests of enrichment and overlap, the hypergeometric test for over enrichment with Bonferroni correction was used. To determine the size of the RNA universe in both hPSCs and neurons, the TPM counts from the RNA-seq data were assessed, and a gene was counted as expressed if it had an average TPM≥1 across five replicates in each FMR1^{y/+} cell type. This generated 15,316 RNAs expressed in neurons, and 14,233 RNAs expressed in hPSCs. These universes were then applied to the developmental disorders, autism, epilepsies and HSA21 gene lists to establish expression. The Allen BrainSpan Atlas of the developing human brain data was used to establish expression in the fetal brain (https://www.brainspan.org/static/home downloaded Dec. 12, 2019). Only prenatal time points were included; all brain regions were included. A gene was considered expressed if the RPKM was ≥1.

Statistical analysis. Replicates for experiments using hPSCs refer to separate wells or plates, and replicates for experiments using neurons refer to independent neuronal differentiations. For eCLIP-seq experiments, peak fold changes were calculated by normalizing usable reads between IP and the SMInput, with a peak threshold of at least 8-fold enrichment in IP over the SMInput and p-values <10⁻³. Enriched p-values were calculated by Chi-square test or Fisher's exact test if the usable read number in IP or SMInput was below 5. For RNA-seq analyses, four to five replicates per genotype and cell type and an adjusted p-value cutoff of 0.05 were used. For mRNA-seq of CRISPRa lines, a log 2foldchange cutoff of over 1 or under −1 was also applied. For Western blot analyses, experiments were performed on triplicate samples and significance was calculated by unpaired two-tailed t-test for comparisons between two groups; Prism (GraphPad Software) was used for statistical analyses. For statistical tests of enrichment and overlap, the hypergeometric test for over enrichment with Bonferroni correction for multiple comparison testing was used. P values (or adjusted P values, where applicable)<0.05 were considered statistically significant.

1. A method of reducing the level of a Dual Specificity Tyrosine Phosphorylation Regulated Kinase 1A (DYRK1A) and/or an amyloid-beta precursor (APP) polypeptide or a polynucleotide encoding such polypeptide in a cell, the method comprising contacting the cell with an expression vector comprising a polynucleotide sequence encoding a Fragile X mental retardation protein (FMRP) polypeptide or a fragment thereof, thereby decreasing the level of the DYRK1A and/or APP protein or polynucleotide in the cell.

2. The method of claim 1, wherein the cell comprises an increased level of DYRK1A and/or APP.

3. (canceled)

4. The method of claim 2, wherein the increased level of DYRK1A and/or APP is associated with a developmental disorder or neurodegenerative disorder.

5. The method of claim 4, wherein the developmental disorder is autism, Fragile X syndrome, or Down syndrome and the neurodegenerative disorder is Alzheimer's disease.

6-9. (canceled)

10. A method for treating a disease associated with an increase in a DYRK1A and/or APP polypeptide in a subject, the method comprising administering an effective amount of a Fragile X mental retardation protein (FMRP) polypeptide or fragment thereof or a polynucleotide encoding said polypeptide or a fragment thereof to the subject.

11. (canceled)

12. The method of claim 10, wherein the increased level of DYRK1A and/or APP is associated with a developmental disorder or neurodegenerative disorder.

13. The method of claim 12, wherein the developmental disorder is autism, Fragile X syndrome, or Down syndrome.

14-21. (canceled)

22. A method of treating a subject having or having a propensity to develop Alzheimer's disease, the method comprising administering to the subject an expression vector comprising a polynucleotide sequence encoding a Fragile X mental retardation protein (FMRP) polypeptide or a fragment thereof, thereby treating Alzheimer's disease.

23. (canceled)

24. The method of claim 22, wherein the subject has Down syndrome.

25. (canceled)

26. A method of treating a subject having or having a propensity to develop Fragile X syndrome, the method comprising administering to the subject an effective amount of a Fragile X mental retardation protein (FMRP) polypeptide or a fragment thereof or a polynucleotide encoding FMRP or a fragment thereof.

27-35. (canceled)

36. A method of treating a disease associated with increased lysine (K)-specific histone demethylase 1A (KDM1A) expression or function and/or with decreased Huntingtin (HTT) expression or function in a subject, the method comprising administering to the subject an effective amount of a Fragile X mental retardation protein (FMRP) polypeptide or a fragment thereof or a polynucleotide encoding FMRP or a fragment thereof.

37. A method of treating a disease associated with increased lysine (K)-specific histone demethylase 1A (KDM1A) expression or function in a subject, the method comprising administering to the subject an effective amount of a Fragile X mental retardation protein (FMRP) polypeptide or a fragment thereof or a polynucleotide encoding FMRP or a fragment thereof.

38. A method of treating a disease associated with decreased Huntingtin (HTT) expression or function in a subject, the method comprising administering to the subject an effective amount of a Fragile X mental retardation protein (FMRP) polypeptide or a fragment thereof or a polynucleotide encoding FMRP or a fragment thereof.

39. The method of claim 36, wherein the increased KDM1A expression or function and/or the decreased HTT expression or function is associated with a developmental disorder or neurodegenerative disorder.

40. The method of claim 39, wherein the developmental disorder is autism, Fragile X syndrome, or Down syndrome and the neurodegenerative disorder is Alzheimer's disease.

41-47. (canceled)

48. A method of decreasing or reducing the expression of a lysine (K)-specific histone demethylase 1A (KDM1A) polypeptide and/or increasing or enhancing the expression of a Huntingtin (HTT) polypeptide or a polynucleotide encoding such polypeptides in a cell, the method comprising contacting the cell with a Fragile X mental retardation protein (FMRP) polypeptide or a fragment thereof, or an expression vector comprising a polynucleotide sequence encoding a Fragile X mental retardation protein (FMRP) polypeptide or a fragment thereof, thereby decreasing or reducing the expression of the KDM1A or polynucleotide, and/or increasing or enhancing the expression of the HTT polypeptide or polynucleotide, in the cell.

49. A method of decreasing or reducing the expression of a lysine (K)-specific histone demethylase 1A (KDM1A) polypeptide or a polynucleotide encoding such polypeptide in a cell, the method comprising contacting the cell with a Fragile X mental retardation protein (FMRP) polypeptide or a fragment thereof, or an expression vector comprising a polynucleotide sequence encoding a Fragile X mental retardation protein (FMRP) polypeptide or a fragment thereof, thereby decreasing or reducing the expression of the KDM1A polypeptide or polynucleotide in the cell.

50. A method of increasing or enhancing the expression of a Huntingtin (HTT) polypeptide or a polynucleotide encoding such polypeptide in a cell, the method comprising contacting the cell with a Fragile X mental retardation protein (FMRP) polypeptide or a fragment thereof or an expression vector comprising a polynucleotide sequence encoding a Fragile X mental retardation protein (FMRP) polypeptide or a fragment thereof, thereby increasing or enhancing the expression of the HTT polypeptide or polynucleotide in the cell.

51-61. (canceled)

62. A method of downregulating expression of a KDM1A polypeptide or polynucleotide encoding KDM1A and/or upregulating expression of an HTT polypeptide or polynucleotide encoding HTT in a cell, wherein increased KDM1A expression and/or decreased HTT expression are associated with a disease or disorder, the method comprising contacting the cell with an expression vector comprising a polynucleotide sequence encoding a Fragile X mental retardation protein (FMRP) polypeptide or a fragment thereof, wherein expression of the KDM1A polypeptide and/or the encoding polynucleotide is downregulated and/or expression of the HTT polypeptide and/or the encoding polynucleotide is upregulated in the cell.

63-71. (canceled)

72. A method of decreasing or reducing the expression of a lysine (K)-specific histone demethylase 1A (KDM1A) polypeptide and/or increasing or enhancing the expression of a Huntingtin (HTT) polypeptide or a polynucleotide encoding such polypeptides in a patient having a developmental or neurodegenerative disease or disorder, the method comprising administering to a patient a Fragile X mental retardation protein (FMRP) polypeptide or a functional fragment thereof, or an expression vector comprising a polynucleotide sequence encoding a Fragile X mental retardation protein (FMRP) polypeptide or a functional fragment thereof, thereby decreasing or reducing the expression of the KDM1A polypeptide or encoding polynucleotide, and/or increasing or enhancing the expression of the HTT polypeptide or encoding polynucleotide, in the patient.

73-77. (canceled)


SEQUENCE LISTING (14 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - DNA, 2378 bp, Homo sapiens
agagcaaggacgcggcggatcccactcgcacagcagcgcactcggtgccccgcgcagggtcgcagatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaactccctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 3 - protein, 763 aa, Homo sapiens
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS

SEQ ID NO: 4 - DNA, 2422 bp, Homo sapiens
gttatagttttgccgctggactcttccctcccttcccccaccccatcaggatgatatgagacttgaaagaagacgatgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcatcaccaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccacaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacgatgaggcaaggggctgatagagaagagtcccccatgacaggagtttgtgtgcaacagagtcctgtagctagctcgtgactacattgaaacttgagtttgtttcttgtgtgtttttatagaagtggtgtttttt

SEQ ID NO: 5 - protein, 632 aa, Homo sapiens [sequence omitted]

SEQ ID NO: 6 - DNA, 4362 bp, Homo sapiens
acggcgagcgcgggcggcggcggtgacggaggcgccgctgccagggggcgtgcggcagcgcggcggcggcggcggcggcggcggcggcggaggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagcttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctacccaactttcaagaaggttctttaataaagcgaaaactcaaccaaatggtacttttccacagtgtaccattaaaatatgcactagtctctttttacaaggctgtattcagcaagggcctaacttgcttaaagtgtaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttt

SEQ ID NO: 7 - protein, 852 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 8 - DNA, 3085 bp, Homo sapiens
ggcgcgtgcgtacgcgacggcggttggcggcgcgcgggcagcgtgaagcgaggcgaggcaaggcttttcggacccacggagcgacagagcgagcggcccctacggccgtcggcggcccggcggcccgagatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagacagatgcattctaagggaagaggcccatgtgcctgtttctgccatgtaaggaaggctcttctagcaatactagatcccactgagaaaatccaccctggcatctgggctcctgatcagctgatggagctcctgatttgacaaaggagcttgcctcctttgaatgacctagagcacagggaggaacttgtccattagtttggaattgtgttcttcgtaaagactgaggcaagcaagtgctgtgaaataacatcatcttagtcccttggtgtgtggggtttttgttttttttttatattttgagaataaaacttcatataaaattg

SEQ ID NO: 9 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 10 - DNA, 13472 bp, Homo sapiens
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaacca

SEQ ID NO: 11 - DNA, 20 bp, Artificial Sequence
  Description of Artificial Sequence Synthetic oligonucleotide
agctcaatggcggtaactcc

SEQ ID NO: 12 - DNA, 20 bp, Artificial Sequence
  Description of Artificial Sequence Synthetic oligonucleotide
gcgctgctgggaaccggccg

SEQ ID NO: 13 - DNA, 20 bp, Artificial Sequence
  Description of Artificial Sequence Synthetic oligonucleotide
caggtcgcactgcctcgcga

SEQ ID NO: 14 - DNA, 20 bp, Artificial Sequence
  Description of Artificial Sequence Synthetic oligonucleotide
agaccagacaccccctcccg